SEQUENCE LISTING (18 sequences)

SEQ ID NO: 1 - DNA, 22 bp, Artificial Sequence
  synthetic construct
cagcacatcagagcagagaaag

SEQ ID NO: 2 - DNA, 24 bp, Artificial Sequence
  synthetic construct
tgttggcatacaggtaataaaagc

SEQ ID NO: 3 - DNA, 23 bp, Artificial Sequence
  synthetic construct
tggatcacagcacatcagagcag

SEQ ID NO: 4 - DNA, 24 bp, Artificial Sequence
  synthetic construct
tggggctcaatctatatctcgaac

SEQ ID NO: 5 - DNA, 24 bp, Artificial Sequence
  synthetic construct
gggaaagcactcacagctaatttg

SEQ ID NO: 6 - DNA, 25 bp, Artificial Sequence
  synthetic construct
gcactggcttaaactgtcattctcc

SEQ ID NO: 7 - DNA, 18 bp, Artificial Sequence
  synthetic construct
gctaacctcaccttcgag

SEQ ID NO: 8 - DNA, 18 bp, Artificial Sequence
  synthetic construct
tgattggacctggttacc

SEQ ID NO: 9 - DNA, 25 bp, Artificial Sequence
  synthetic construct
gccaagcagcagtacattctacacg

SEQ ID NO: 10 - DNA, 25 bp, Artificial Sequence
  synthetic construct
gctgttcttcacgtgctcaaaatcc

SEQ ID NO: 11 - DNA, 21 bp, Artificial Sequence
  synthetic construct
gatgttgaggtacagaatcgt

SEQ ID NO: 12 - DNA, 18 bp, Artificial Sequence
  synthetic construct
ggtcggtctggatggcga

SEQ ID NO: 13 - DNA, 20 bp, Artificial Sequence
  synthetic construct
cagacccactcagatgtcaa

SEQ ID NO: 14 - DNA, 20 bp, Artificial Sequence
  synthetic construct
catagttagtcacacctcgt

SEQ ID NO: 15 - DNA, 18 bp, Artificial Sequence
  synthetic construct
ggactcggtggacttctc

SEQ ID NO: 16 - DNA, 18 bp, Artificial Sequence
  synthetic construct
cgcatctcctcctcgtag

SEQ ID NO: 17 - DNA, 57 bp, Artificial Sequence
  synthetic construct
ccggcccataaagtgagtctgtcctctcgagaggacagactcactttatgggttttt

SEQ ID NO: 18 - DNA, 501 bp, Homo sapiens
caaggaattaaaaacatgtataaaacattgttgtataaaaacccattaaaaagaatggaagacactatagtaaaatcattgtgggtttagttgttataacacattttaaaaatctttgatcccaatcaatatttataagaaagaagaaatatggaattatttcctgagtcaaggagcagggagagaatgaggaagaagaggaggaggaggagggggaggaggagacaataaacctacttcccaaagttaacaaacaaaaagtgggaagaggtcaaagactacaaggagtagaattaacgtcaattgtttctatgtttgagtctgaaaattttttgtcccttctccaccaacctatatattgatacacatataaatgctaaaggcatttttgaatttgaacagatcattttctttgtatggctgcctttaaaaaaaattcaacctggtcactcttcctcaacatttactgaggtctaagtgttcaatttagaacacatgctt

(19) United States
(12) Patent Application Publication
(10) Pub. No.: US 2013/0263297 A1
(43) Pub. Date: Oct. 3, 2013

(54) METHODS OF TREATING CANCER

(71) Applicant: Cedars-Sinai Medical Center, Los Angeles, CA (US)

(72) Inventors: Gina C.Y. Chu, Los Angeles, CA (US); Leland W.K. Chung, Beverly Hills, CA (US); Haiyen E. Zhau, Beverly Hills, CA (US); Ruoxiang Wang, Los Angeles, CA (US)

(21) Appl. No.: 13/889,135

(22) Filed: May 7, 2013

Related U.S. Application Data
(63) Continuation-in-part of application No. PCT/US11/60204, filed on Nov. 10, 2011.
(60) Provisional application No. 61/718,626, filed on Oct. 25, 2012.
(60) Provisional application No. 61/412,360, filed on Nov. 10, 2010.
(60) Provisional application No. 61/442,679, filed on Feb. 14, 2011.

(51) Int. Cl. A61K 39/395; A61K 31/4545; A61K 31/404; A61K 31/713

(52) U.S. Cl. CPC A61K 39/39558; A61K 31/713; A61K 31/4545; A61K 31/404
USPC 800/10; 514/44 A; 424/142.1; 424/174.1; 424/158.1; 514/318; 514/414; 435/375; 435/325; 435/7.23; 435/6.12

(57) ABSTRACT

The present invention relates to metastases. More specifically, the invention relates to compositions and methods for the inhibition of metastases of cancer cells, such as prostate cancer cells, to bones and soft tissue. The present invention also relates to the treatment of cancer, including but not limited to prostate cancer. Also provided are animal models for studying cancer metastasis; particularly, prostate cancer metastasis. Further provided are compositions, processes and systems to prognosticate cancer.

### STATEMENT REGARDING FEDERALLY-SPONSORED RESEARCH

The U.S. Government has a paid-up license in this invention and the right in limited circumstances to require the patent owner to license others on reasonable terms as provided for by the terms of Grant No. 2P01CA098912 awarded by National Institutes of Health.

### FIELD OF INVENTION

This invention relates to cancer prognosticating, cancer treatment and animal models based on the understanding of mechanisms of cancer progression supported by both clinical and animal models of cancer bone and soft tissue metastases.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Advanced prostate cancer frequently causes bone metastases (Li et al., 2006), and about 80% of prostate cancer patients (Landis, Murray, Bolden, & Wingo, 1999) will have tumors metastasize to bone, which is usually incurable. In addition, approximately 350,000 people die with bone metastases annually in the United States (Mundy, 2002). Bone metastasis is a result of dysregulation of the normal bone remodeling process as results from cancer migration, invasion and colonization in the skeleton: first, bone resorption by osteoclasts, and then bone formation by osteoblasts at the same site. Osteoclaststogenesis is a process that resorbs bone by secreting proteases that dissolve the bone matrix, and producing acid that releases bone mineral into the extracellular space under the ruffled border of the plasma membrane of osteoclasts (Blair, Teitelbaum, Ghiselli, & Gluck, 1989). Moreover, the bone resorptive process is highly dependent on the adherence of osteoclasts to the bone surface. Osteoclasts arise from precursor cells in the monocyte macrophage lineage, which differentiate into mature osteoclasts (Roodman, 1999). RANKL, a receptor activator of nuclear factor-KB (NF-KB) ligand, plays an essential role in the osteoclastogenesis. RANKL is a member of the family of tumor necrosis factors, which is expressed on the surface of osteoblasts and stromal cells and is released by activated T cells (Roodman, 2004). Osteotropic factors, 1,25-dihydroxyvitamin D₃, parathyroid hormone (PTH), prostaglandin E-2, and interleukin-1, induce the formation of osteoclasts by up-regulating the expression of RANKL on the surface of marrow stromal cells and osteoblasts rather than by activating directly on the osteoclast precursors (Yasuda et al., 1998). RANKL then binds to the RANK receptors on the surface of osteoclast precursors and signals through the NF-KB and Jun N-terminal kinase (JNK) pathways to induce the formation of mature osteoclasts and promote osteoclast survival. Activated T cells can also produce cytokines to either inhibit (i.e., interleukin-4, -18, and γ) or stimulate (i.e. interleukin-17) the formation of osteoclasts (Roodman, 2004). Osteoprotegerin (OPG) is another key regulator of bone metabolism in both normal and metastatic state. OPG is a decoy receptor of RANKL produced by osteoblast/stromal cells, and it acts as an antagonist to bind to RANKL and inhibit the formation of osteoclasts. Therefore, the balance of RANKL/RANK/OPG triad system represents a key regulatory mechanism in osteoclastogenesis, and an imbalanced state of RANKL/OPG expression has been implicated in bone metastases in prostate cancer (Chen et al., 2006). Studies have also revealed that expression of RANKL/RANK/OPG was low in normal but markedly higher in prostate cancer cell lines (Chen et al., 2006).

However, the characterization of RANKL/RANK and OPG as well as the underlying mechanisms of bone metastases in prostate cancer are still limited and not fully determined. This is in part due to the lack of the full understanding of the biology of invading cancer cells in the bone microenvironment and how cancer cells may participate in bone turnover through interference of RANKL/RANK/OPG triad system and the abundant soluble and insoluble factors in the bone matrices that could affect ultimately the fate of cancer cells and their interactions with RANKL/RANK/OPG triad system. Therefore, the inventors' studies investigate the regulation of RANKL at both transcriptional and translational levels using the human ARCaP and LNCaP cell lines as the cell model system, which are established respectively from the ascites fluid or lymph nodes of patients with metastatic prostate cancer (Xu et al., 2006; Thalmann, et al. 1994). The reasons to select these models include: 1) ARCaP human prostate cancer cells harbor wild type androgen receptor, is androgen-refractory and exhibit consistently aggressive bone metastatic behaviors upon epithelial to mesenchymal transition (EMT). These cells secrete low level of prostatic specific antigen as compared to other prostate cancer cell lines; 2) LNCaP is an androgen-responsive, marginally tumorigenic and non-metastatic human prostate cancer cell line. This cell line fails to grow in castrated hosts, also without ability to colonize bone and soft tissues.

Prostate cancer (PCa) is the second leading cause of cancer death in American men. Most men who die of PCa develop castration-resistant bone metastatic disease, which presently has no cure. Bone metastasis is also associated with hypercalcemia, anemia, bone pain, recurrent infection, spinal cord compression, fractures and paralysis [1, 2]. There is a need to understand the multi-step program of carcinogenesis at the molecular level to develop pathway-based novel therapeutics for personalized targeting at the primary and metastatic sites, since tumor genetic constituents and behavioral anomalies are now shown to be regulated by interactions between cancer cells and their microenvironment [3, 4]. The molecular mechanism of PCa bone colonization remains elusive, but it is now well established that PCa cells can home to bone but not necessarily colonize to bone successfully, PCa cells must interact reciprocally with bone cells by promoting bone resorption and new bone formation [4-6].

The development of an effective therapy for PCa metastasis is hampered by a lack of robust bone and soft tissue metastasis animal models closely mimicking human disease progression, and the absence of a clinically promising therapeutic target.

As such, there remains a need in the art for additional therapies and research models for the treatment of cancer.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method for treating cancer in a mammalian subject in need thereof, comprising: providing an agent capable of inhibiting RANK and/or RANKL, and an agent capable of inhibiting HGF-c-Met/VEGFR2/neuropilin-1-mediated signaling; and administering the agent capable of inhibiting RANK and/or RANKL and the agent capable of inhibiting HGF-c-Met/VEGFR2/neuropilin-1-mediated signaling to the mammalian subject to treat cancer.

In various embodiments, the agent capable of inhibiting RANK and/or RANKL can be provided in a first composition and the agent capable of inhibiting HGF-c-Met/VEGFR2/neuropilin-1-mediated signaling can be provided in a second composition. In various embodiments, the agent capable of inhibiting RANK and/or RANKL and the agent capable of inhibiting HGF-c-Met/VEGFR2/neuropilin-1-mediated signaling can be provided in one composition.

In various embodiments, the agent capable of inhibiting RANK and/or RANKL can be denosumab, RANK-Fc, OPG-Fc, shRNA, or siRNA. In certain embodiments, the shRNA or the siRNA inhibits RANKL expression. In certain embodiments, the agent capable of inhibiting RANK and/or RANKL can be denosumab.

In various embodiments, the agent capable of inhibiting HGF-c-Met/VEGFR2/neuropilin-1-mediated signaling can be denosumab, RANK-Fc, OPG-Fc, shRNA, siRNA, XL-184, crizotinib, or VEGFR2 kinase inhibitor III (CAS 204005-46-9). In certain embodiments, the shRNA or the siRNA inhibits RANKL expression. In certain embodiments, the agent capable of inhibiting HGF-c-Met/VEGFR2/neuropilin-1-mediated signaling can be XL-184.

In various embodiments, the cancer can be prostate, kidney, breast, bladder, lung, breast, ovarian, pancreatic, thyroid, liver, gastric, colon or melanoma. In certain embodiments, the cancer can be prostate cancer.

In various embodiments, inhibiting HGF-c-Met/VEGFR2/neuropilin-1-mediated signaling can comprise inhibiting activation of c-Met, VEGFR2, neuropilin-1, Src-kinase, Stat3, Mcl-1, NF-kB and combinations thereof.

Various embodiments of the present invention provide for a method of preventing, reducing the likelihood of and/or inhibiting metastases of cancer cells, comprising: providing a composition comprising an agent capable of inhibiting epithelial-to-mesenchymal transition (EMT) of cancer cells; and administering a quantity of the composition to the a mammalian subject in need thereof to prevent, reduce the likelihood of and/or inhibit metastases of cancer cells.

In various embodiments, the agent capable of inhibiting EMT can be osteoprotegerin (OPG) and can bind to RANKL to inhibit the formation of osteoclasts, thereby preventing, reducing the likelihood and/or inhibiting metastases of the cancer cells.

In various embodiments, the agent capable of inhibiting EMT can be denosumab, RANK-Fc, OPG-Fc, siRNA, shRNA, XL-184, crizotinib, VEGFR2 kinase inhibitor III (CAS 204005-46-9) or combinations thereof. In various embodiments, the siRNA or the shRNA can inhibit RANKL expression.

In various embodiments, the cancer cells can be prostate, kidney, breast, bladder, lung, ovarian, pancreatic, thyroid, liver, gastric, colon or melanoma cancer cells. In various embodiments, the cancer cells can be prostate cancer cells.

Various embodiments of the present invention provide for a method of inhibiting a process of RANKL-mediated awakening of cancer dormancy, comprising: providing a composition comprising an agent capable of inhibiting epithelial-to-mesenchymal transition (EMT) of cancer cells; and administering a quantity of the composition to the a mammalian subject in need thereof to inhibiting the process of RANKL-mediated awakening of cancer dormancy.

In various embodiments, the agent capable of inhibiting EMT can be osteoprotegerin (OPG) and can bind to RANKL to inhibit the formation of osteoclasts, thereby inhibiting the process of RANKL-mediated awakening of cancer dormancy.

In various embodiments, the agent capable of inhibiting EMT can be denosumab, RANK-Fc, OPG-Fc, siRNA, shRNA, XL-184, crizotinib, VEGFR2 kinase inhibitor III (CAS 204005-46-9) or combinations thereof. In various embodiments, the siRNA or the shRNA can inhibit RANKL expression. In various embodiments, the cancer can be prostate, kidney, breast, bladder, lung, ovarian, pancreatic, thyroid, liver, gastric, colon or melanoma cancer. In various embodiments, the cancer can be prostate cancer.

Various embodiments of the present invention provide for a cell expressing a target selected from the group consisting of RANKL, an EMT marker, NF-kB, c-Met, VEGFR2, neuropilin-1, Src-kinase, Stat3, Mcl-1, and combinations thereof.

Various embodiments of the present invention provide for a method of identifying a compound that inhibits metastasis, comprising: providing the cell expressing a target selected from the group consisting of RANKL, an EMT marker, NF-kB, c-Met, VEGFR2, neuropilin-1, Src-kinase, Stat3, Mcl-1, and combinations thereof; contacting the cell with a test compound; and determining whether metastasis is inhibited in the presence of the test compound, wherein the decrease of the expression of the target can be an indication that the test compound inhibits metastasis or wherein the decrease of a target's upstream signaling components, Src-kinase or Stat3 phosphorylation can be an indication that the test compound inhibits metastasis.

In various embodiments, the EMT marker can be selected from the group consisting of N-cadherin, vimentin, VEGF, RANKL, c-Met and combinations thereof.

In various embodiments, the cell can be a cell overexpressing the target. In various embodiments, the cell can be a prostate, kidney, breast, bladder, lung, ovarian, pancreatic, thyroid, liver, gastric, colon or melanoma cancer cell. In various embodiments, the cell can be a prostate cancer cell. In various embodiments, the prostate cancer cell can be ARCaP_{E}, ARCaP_{M}, C4-2, LNCaP, PC3 or MCF7. In certain embodiments, the cell is an LNCaP-RNAKL cell.

Various embodiments of the present invention provide for an animal, comprising the cell expressing a target selected from the group consisting of RANKL, an EMT marker, NF-kB, c-Met, VEGFR2, neuropilin-1, Src-kinase, Stat3, Mcl-1, and combinations thereof.

Various embodiments of the present invention provide for a method of identifying a compound that inhibits metastasis, comprising: providing the animal comprising the cell expressing a target selected from the group consisting of RANKL, an EMT marker, NF-kB, c-Met, VEGFR2, neuropilin-1, Src-kinase, Stat3, Mcl-1, and combinations thereof; contacting the animal with a test compound; and determining whether metastasis is inhibited in the presence of the test compound. In various embodiments, the cell can be a prostate, kidney, breast, bladder, lung, ovarian, pancreatic, thyroid, liver, gastric, colon or melanoma cancer cell. In various embodiments, the cell can be a prostate cancer cell. In various embodiments, the prostate cancer cell can be ARCaP_{E}, ARCaP_{M}, C4-2, LNCaP, PC3 or MCF7. In certain embodiments, the cell is an LNCaP-RNAKL cell. In various embodiments, the animal is a mouse.

In various embodiments, the decrease of the expression of the target can be an indication that the test compound inhibits metastasis or the decrease of the target's upstream signaling components, Src-kinase or Stat3 phosphorylation can be an indication that the test compound inhibits metastasis.

In various embodiments, the decrease of metastasis of a cancer in the animal can be an indication that the test compound inhibits metastasis.

Various embodiments of the present invention provide for a method of switching osteolytic bone lesion and/or metastasis to osteoblastic bone lesion and/or metastasis in a subject in need thereof comprising: providing an agent capable of blocking RANK/RANKL signaling and/or HGF/cMet/VEGF/VEGFR2/neuropilin-1-mediated signaling; and administering the agent to the subject to switch osteolytic bone lesion and/or metastasis to osteoblastic bone lesion and/or metastasis.

In various embodiments, agent can be selected from the group consisting of denosumab, RANK-Fc, OPG-Fc, siRNA, shRNA, XL-184, crizotinib, VEGFR2 kinase inhibitor III (CAS 204005-46-9) and combinations thereof. In various embodiments, the siRNA or the shRNA can inhibit RANKL expression.

Embodiments of the present invention provide for a process, comprising: providing a composition comprising one or more detection probes to detect a protein selected from the group consisting of RANK, RANKL, and combinations thereof; contacting the first composition to a tissue sample or a cell sample from a mammalian subject desiring a determination of cancer survival; quantifying the level RANK or RANKL to determine the likelihood of cancer survival in the mammalian subject. In various embodiments, the protein can be RANKL.

In various embodiments, the one or more detection probe can comprise a label to produce a signal to detect the protein. In various embodiments, the label can be selected from the group consisting of a radiolabel, a chromophore, a fluorophore, a quantum dot and combinations thereof.

In various embodiments, the process can further comprise predicting a long survival time when a level of RANK or RANKL is lower than an established average, or predicting a short survival when a level of RANK or RANKL is higher than an established average. In various embodiments, the process can further comprise predicting a survival of over 100 months when a level of RANK or RANKL is lower than an established average, or predicting a of surviving less than 100 months when a level of RANK or RANKL is higher than an established average.

In various embodiments, the level of RANK or RANKL can be used to determine a course of therapy. In various embodiments, the course of therapy can comprise administering an agent capable of blocking RANK/RANKL signaling and/or HGF/cMet/VEGF/VEGFR2/neuropilin-1-mediated signaling. In various embodiments, the agent capable of blocking RANK/RANKL signaling and/or HGF/cMet/VEGF/VEGFR2/neuropilin-1-mediated signaling can be selected from the group consisting of denosumab, RANK-Fc, OPG-Fc, siRNA, shRNA, XL-184, crizotinib, VEGFR2 kinase inhibitor III (CAS 204005-46-9) and combinations thereof.

In various embodiments, the tissue sample or cell sample can be obtained from a tumor.

In various embodiments, the one or more detection probes can be an antibody capable of binding to RANK, RANKL, or combinations thereof.

Various embodiments of the present invention provide for a process, comprising providing a first composition comprising one or more isolated nucleic acid probes; contacting the first composition to an RNA sample from a mammalian subject desiring a determination of cancer survival, to produce one or more cDNA molecules; providing a second composition comprising one or more isolated nucleic acids probes comprising a sequence capable of hybridizing to one or more nucleic acids selected from the group of genes consisting of RANK and RANKL; contacting the second composition with the one or more cDNA molecules to amplify the one or more cDNA molecules; and quantifying the expression level of the one or more genes to determine the likelihood of cancer survival in the mammalian subject. In various embodiments, the one or more genes can be RANKL.

In various embodiments, the process can further comprise predicting a long survival time when an expression level of the one or more genes is lower than an established average, or predicting a short survival when an expression level of the one or more genesis higher than an established average. In various embodiments, the process can further comprise predicting a survival of over 100 months when an expression level of the one or more genes is lower than an established average, or predicting a of surviving less than 100 months when an expression level of the one or more genes is higher than an established average.

In various embodiments, the expression level of the one or more genes can be used to determine a course of therapy. In various embodiments, the course of therapy can comprise administering an agent capable of blocking RANK/RANKL signaling and/or HGF/cMet/VEGF/VEGFR2/neuropilin-1-mediated signaling. In various embodiments, the agent capable of blocking RANK/RANKL signaling and/or HGF/cMet/VEGF/VEGFR2/neuropilin-1-mediated signaling can be selected from the group consisting of denosumab, RANK-Fc, OPG-Fc, siRNA, shRNA, XL-184, crizotinib, VEGFR2 kinase inhibitor III (CAS 204005-46-9) and combinations thereof.

In various embodiments, the RNA sample can be obtained from a tumor sample.

Various embodiments of the invention provide for a composition, comprising: one or more detection probes to detect a protein selected from the group consisting of RANK, RANKL, and combinations thereof; and a tissue sample or a cell sample from a mammalian subject desiring a determination of cancer survival. In various embodiments, the protein can be RANKL.

In various embodiments, the one or more detection probes can comprise a label to produce a signal to detect the protein. In various embodiments, the label can be selected from the group consisting of a radiolabel, a chromophore, a fluorophore, a quantum dot and combinations thereof.

In various embodiments, the one or more detection probes can be an antibody capable of specifically binding to RANK, RANKL, or combinations thereof.

Various embodiments of the invention provide for a composition, comprising: one or more isolated nucleic acids probes comprising sequences capable of hybridizing to the group of genes consisting of RANK and RANKL; and a tissue sample or a cell sample from a mammalian subject desiring a determination of cancer survival.

In various embodiments, the composition can further comprise: one or more isolated cDNA molecules transcribed from an RNA sample obtained from a mammalian subject desiring a determination of cancer survival.

Various embodiments of the invention provide for an array, comprising: a substrate having a composition comprising one or more isolated nucleic acids probes comprising sequences capable of hybridizing to the group of genes consisting of RANK and RANKL; or one or more isolated nucleic acids probes comprising sequences capable of hybridizing to the group of genes consisting of RANK and RANKL and one or more isolated cDNA molecules transcribed from an RNA sample obtained from a mammalian subject desiring a prognosis regarding a cancer.

Various embodiments of the invention provide for a process of prognosticating a tumor, comprising: providing one or more probes to detect the expression level of one or more genes or level one or more proteins from the group consisting of RANK, RANKL and combinations thereof; contacting the one or more probes to a test sample obtained from a mammalian subject desiring a prognosis regarding a tumor; determining one or more expression levels of the one or more genes or one or more proteins; prognosticating the tumor based on the one or more expression levels of the one or more genes or the level of the one or more proteins. In various embodiments, the one or more genes or one or more proteins can be RANKL.

In various embodiments, the one or more probes can be an antibody capable of specifically binding to RANK, RANKL, or combinations thereof, and determining one or more expression levels can be for the one or more proteins.

In various embodiments, the one or more detection probes can comprise a label to produce a signal to detect the protein. In various embodiments, the label can be selected from the group consisting of a radiolabel, a chromophore, a fluorophore, a quantum dot and combinations thereof.

In various embodiments, the process can further comprise predicting a long survival time when an expression level of the one or more genes or the level of the one or more protines is lower than an established average, or predicting a short survival when an expression level of the one or more genes or the level of the one or more proteins is higher than an established average. In various embodiments, the process can further comprise predicting a survival of over 100 months when an expression level of the one or more genes or level of the one or more proteins is lower than an established average, or predicting a of surviving less than 100 months when an expression level of the one or more genes or level of the one or more proteins is higher than an established average.

Various embodiment provide for a system, comprising: an isolated sample obtained from a mammalian subject desiring the prognosis of a cancer, the isolated sample comprising an abnormal level of RANK, RANKL, or combinations thereof; and a reagent to react with RANK, RANKL, or combinations thereof. In various embodiments, the isolated sample can comprise an abnormal level of RANKL and the reagent can be a reagent to react with RANKL.

In various embodiments, the reagent can further comprise a label to produce a signal to detect the abnormal level of RANK, RNAKL or combinations thereof. In various embodiments, the label can be selected from the group consisting of a radiolabel, a chromophore, a fluorophore, or a quantum dot.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts RT-PCR of RANKL, RANK, and OPG expression in prostate cancer cell accordance with various embodiments of the present invention.

FIG. 2 depicts RANKL treated with ARCaP_{E} cells and EMT markers examined by RT-PCR and WB in accordance with various embodiments of the present invention.

FIG. 3 depicts ARCaP_{E} cells treated with RANKL and RANKL+OPG in accordance with various embodiments of the present invention.

FIG. 4 depicts RANKL overexpression in ARCaP_{E} cells in accordance with various embodiments of the present invention.

FIG. 5 depicts transient knockdown of RANKL in ARCaP_{M} cells in accordance with various embodiments of the present invention.

FIG. 6 shows that RANKL expression is expressed in clinical prostate cancer and cancer bone metastasis in accordance with various embodiments of the present invention. RANKL, RANK, and OPG are differentially expressed in human prostate cancer cell lines. (A) RANKL expression was examined in clinical human prostate cancer specimen by immunohistochemical (IHC) staining RANKL expression is negatively stained in the benign tumor but positively stained in the well- and poorly-differentiated primary human prostate tumors as well as in prostate tumors metastasized to the skeleton. There is an indication that RANKL expression may be correlated with human prostate cancer progression and bone metastasis. (B) RANKL/RANK/OPG expression was examined in different isogenic human prostate cancer lines (ARCaP_{E}, ARCaP_{M}, LNCaP, C4-2, C4-2B, PC-3, PC-3M, DU-145) and human osteoblastic tumor cell lines (MG-63 and SaoS-2). The expression was shown by both RT-PCR (Panel B) and western blot (WB, Panel B).

FIG. 7 shows that RANKL treatment induces morphological and biochemical changes of prostate cancer cells to undergo EMT progression, leading to increased in vitro migration and invasion in ARCaP_{M} cells in accordance with various embodiments of the present invention. Addition of OPG can prevent RANKL-induced EMT transformation of prostate cancer cells. (A) ARCaP_{E} cells were serum-starved for overnight and treated with 200 ng/ml of trimerized recombinant RANKL protein (rRANKL) for 8 days. Cell morphological changes were observed at day 2, 5, and 8 and images were taken with light microscopy (20× magnification). (B1, B2, and B5) Prostate cancer progression model, ARCaP and LNCaP cells were treated with 200 ng/ml of rRANKL proteins alone or in the presence of OPG, a decoy RANK receptor, biochemical changes of EMT-specific markers were examined by RT-PCR and Western blot analyses. RANKL decreased epithelial marker E-cadherin and increased mesenchymal marker vimentin, N-cadherin, and Snail. RANKL also increased its own expression. Addition of OPG abolished RANKL-induced biochemical EMT changes. (B3 and B4) The mesenchymal ARCaP_{M} cells when treated with 1 μg/ml of OPG or transiently knocked down RANKL showed increased expression of epithelial marker but decreased expression of mesenchymal markers, indicating a reversal of EMT phenotype. A pool of 3 target-specific 20-25 nt siRNAs designed to knock down RANKL gene was used. (C) RANKL protein treatment enhanced the migration and invasion of ARCaP_{E} cells and LNCaP cells, which could be diminished in the presence of OPG. Consistently with these findings, ARCaP_{M} cells when treated with 1 μg/ml of OPG, showed decreased migration and invasion potential.

FIG. 8 shows that RANKL overexpression also promoted EMT progression of prostate cancer cells and increased their migration and invasion potential in accordance with various embodiments of the present invention. (A) Characterization of RANKL overexpression in ARCaP_{E} cells and LNCaP cells by Western blot analysis. RANKL expression constructs were tagged with GFP or Flag proteins, and RANKL overexpression was detected by RANKL, GFP, or Flag antibodies. (B) RANKL overexpression changed the morphology of ARCaP_{E} and LNCaP cells detected under light microscopy (10× and 20× magnification). Since RANKL expression construct introduced into the ARCaP_{E} cells has a GFP tag, RANKL overexpression was also detected using the fluorescent microscopy and RANKL was shown to be expressed on the membrane, cytosol and nuclei of the genetically tagged cells (10× and 20× magnification). (C) RANKL overexpression also induced EMT changes at molecular level. (D) Stable clones overexpressing RANKL or Neo constructs were established in both ARCaP_{E} and LNCaP cells, and their proliferation rates were examined using MTS assay. RANKL was not found to affect the basal cell proliferation rate of both ARCaP_{E} and LNCaP cells. (E) RANKL overexpression, however, increased cell migration and invasion of ARCaP_{E} and LNCaP cells, which were antagonized upon addition of OPG.

FIG. 9 shows that RANKL expressed in ARCaP_{E} and LNCaP cells was found to be biologically active driving the maturation of cancer cell-adjacent osteoclast precursor cells in a paracrine fashion in accordance with various embodiments of the present invention. (A) RANKL-expressing ARCaP_{E} and LNCaP cells were co-cultured with osteoclast precursor RAW-264.7 cells in the presence or absence of OPG for five days, and TRAP+ multinucleated mature osteoclasts were stained and counted under light microscopy. Addition of 100 ng/ml of RANKL protein to RAW264.7 cells was served as positive control. RANKL overexpressed by ARCaP_{E} and LNCaP cells induced five folds more TRAP+ mature osteoclasts compared to that induced by Neo control cells, indicating that RANKL expressed by ARCaP_{E} and LNCaP cells were functional. (B) Representative images of TRAP+ multinucleated mature osteoclasts induced by RANKL protein, in ARCaP_{E}-RANKL and LNCaP-RANKL as well as the Neo control cells in the presence or absence of OPG (20× magnification).

FIG. 10 shows that RANKL induced EMT in prostate cancer cells by activating NF-κB signaling through AKT and p38 dependent pathways in accordance with various embodiments of the present invention. (A) RANKL activated PI3K-AKT, p38, and NF-KB by increasing their phosphorylation levels in ARCaP_{E} and LNCaP cells detected by Western blot analysis. (B) ARCaP_{E}-RANKL and LNCaP-RANKL cells were treated with PI3K (20 μM LY294002), p38 (20 μM, SB203580), and NF-KB (2 μM, PS341) inhibitors for 4 hours, and the cells were harvested for cell lysis followed by Western blot analysis.

FIG. 11 shows that RANKL increased the metastatic potential of both LNCaP and ARCaP (Table 3). RANKL expressing PCa cells induced osteolytic lesions in bone (11A). Representative images of metastatic tumors in bone and soft tissues induced by LNCaP-RANKL cells inoculated in nude mice (11A). RANKL increased the metastatic potential of both LNCaP and ARCaP_{E} cells and induced osteolytic lesions in bone. Representative 3D μCT scans demonstrate osteolytic lesions in mouse femur, spine, jaw, skull, and tibia induced by the LNCaP-RANKL and ARCaP_{E}-RANKL tumor cell inoculation in nude mice (11B). RANKL-induced tumors expressed mesenchymal phenotype and exhibited increased osteoclastic activity in bone (11C). Immunohistochemical (IHC) staining of RANKL and EMT marker expression in bone and soft tissue (lymph nodes) tumors induced by LNCaP-RANKL cells inoculated in nude mice (11C). TRAP staining of mature osteoclast lining (pink red) in bone tumor induced by the inoculation of LNCaP-RANKL cells in nude mice compared to the normal bone surface from nude mice inoculated with LNCaP-Neo cells where no TRAP-positive osteoclasts were detected (11D). Images were taken with light microscopy with 25× magnification.

FIG. 12 shows that small numbers of RANKL-expressing PCa cells conferred tumorigenicity of RANKL-non-expressing PCa cells in bone in accordance with various embodiments of the present invention. One thousand, ten thousands, and hundred thousands of RANKL-expressing LNCaP (LN-RANKL) cells were co-inoculated with a million of RANKL non-expressing LNCaP cells tagged with red fluorescent protein (LN-RFP) in both tibia of nude mice. The inventors noted tumor formation at both tibial and soft tissues sites with the latter due to metastasis from mouse tibia. Tibial and soft tissue tumors were harvested and subjected to fluorescent imaging for detection of the red fluorescent signal. The results showed that as low as a thousand LN-RANKL cells can promote the non-tumorigenic, RANKL-non-expressing LN-RFP cells to grow tumors in the tibia of mice. As the number of LN-RANKL cells increased, the tibial tumors were bigger and the red fluorescent signal detected were stronger, implicating that the RANKL-expressing LNCaP cells augmented the tumorigenic potential of RANKL-non expressing LN-RFP cells in the tumor microenvironment.

FIG. 13 demonstrates RANKL-expressing LN Cells Promote Co-colonization of Non-metastatic and RANKL-non-expressing LN Cells to the Metastatic Sites in accordance with various embodiments of the present invention. (A) Representative images and corresponding red fluorescent signal intensity of bone and soft tissue tumors harvested from nude mice bearing tumors of mixed LN-RANKL plus LN-RFP cells at 1 to 9 and 9 to 1 ratios and intratibial inoculation of LN-RANKL cells followed by intracardiac inoculation of LN-RFP cells. Metastatic bone and soft tissue tumors from all three groups showed the participation of red fluorescent signals, especially the bone tumors compared to the normal organs, such as spleen, bone, and kidney, which serve as negative controls. Representative in vivo X-ray and fluorescent images (Ex: 740; Em: 840) of intratibial inoculation of LNCaP-RFP cells followed by intracardiac inoculation of LNCaP-RANKL cells. (B) Representative cells expressed red fluorescent signals detected from frozen sections of bone and soft tissue tumors harvested from each group (20× magnification) by a fluorescent microscopy. Parts of the tumors were fixed in OCT and sectioned using Cryostat at −25° C. (C) Representative images of red fluorescent microscopy and corresponding immunohistochemical staining of RFP and hematoxylin and eosin (H&E) staining from paraffin-embedded tumors of each group detected by light and fluorescent microscopy (25× magnification). (D) Representative images of a single quantum dot (QD) labeling of RANKL (green) and RFP (red) as well as the merged QD labeling of paraffin-embedded tumor sections counterstained with DAPI nuclear staining (blue). The QD labeling showed that the number of colonizing LN-RFP cells corresponds with the number of co-inoculated LN-RFP cells in mice. It also showed that RANKL expression in LN-RANKL induced bone tumors is heterogeneous, implicating the possibility that RANKL expression can be switched on/off by the prostate cancer cells depending on the surrounding microenvironment.

FIG. 14 shows that co-culture of RANKL-expressing and non-expressing PCa cells induces increased osteoblastic activity in vitro and mixed osteolytic and osteoblastic lesions in vivo in accordance with various embodiments of the present invention. In vitro osteogenic assay (A) and in vitro osteoclastogenesis assay (B) show that LN-RANKL cells induced high levels of osteoclast differentiation but low level of osteoblast mineralization compared to that induced by LN-Neo or LN-RFP cells. Nevertheless, upon co-culture with LN-RFP cells, the number of mature osteoclast formed decreased and the osteoblast mineralization increased significantly compared to the LN-RANKL cells alone. The levels of osteoclast maturation and osteoblast mineralization were comparable between the LN-Neo, or LN-RFP cells as well as the co-culture of either of these cell types with LN-RANKL cells (FIGS. 14A and 14B). These results implicate that the presence of LNCaP cells can switch predominantly osteoclastic LN-RANKL cells to become more osteoblastic-like when co-cultured with either LN-Neo or LN-RFP cells. (C) Representative 3D μCT scans demonstrate that mice bearing either 1 to 9 or 9 to 1 ratio of LN-RANKL and LN-RFP cells or intratibial LN-RANKL plus intracardial LN-RFP cells all displayed mixed osteolytic and osteoblastic lesions in the bone. Intratibial co-inoculation of a thousand, ten thousands, and hundred thousands of LNCaP-RANKL cells plus LNCaP-RFP cells also led to mixed osteolytic and osteoblastic lesions (D) TRAP staining of mature osteoclast lining (red) and orange G staining of new bone formation (yellow orange) in bone tumor induced by the co-inoculation of LN-RANKL and LN-RFP cells in nude mice. Bone tumors from all three groups exhibited osteolytic as well as osteoblastic activities at bone and tumor interface. Higher osteoblastic but lower osteolytic activity was observed in the bone tumors induced by 1 to 9 ratio of LN-RANKL and LN-RFP cells as well as by the intratibial LN-RANKL plus intracardial LN-RFP cells (E) Alternative evidence of the increased level of osteoblastic activity in the tumors induced by the co-inoculation of LN-RANKL and LN-RFP cells is illustrated by detecting the osteoid formation/thickness using Trichrome staining. The osteoid stained in red, and the mineralized bone stained in turquoise. The level of osteoid formation and thickness can reflect the level of bone formation. (Ott, 2008)

FIG. 15 shows that RANKL promotes PCa cell EMT in accordance h various embodiments of the present invention. Stable transfection was used to study the role of RANKL in promoting PCa cell EMT. (A) In both LNCaP (LN) and ARCaP_{E} cells, RANKL overexpression was accompanied by cell's transition to mesenchymal morphology. As noted, the morphologic transition is much more prominent in ARCaP than LNCaP model. (B) RANKL overexpressing LNCaP cells (LN-RANKL) display specific expressional changes indicative of EMT, with increased levels of mesenchymal markers N-cadherin, vimentin and Snail, but with decreased epithelial E-cadherin. Note that the overexpression induced endogenous RANKL expression (i.e., both flag-tagged and endogenous). Expression of c-Met is also induced in RANKL overexpressing cells. (C) RANKL and HGF treatments also induced c-Met expression and phosphorylated levels of c-Met in LNCaP cells. Such induction can be abolished upon addition of OPG and HGF neutralizing antibody, respectively. The induced c-Met is biologically functional since the p-c-Met expression increased upon the addition of HGF, and this activation of p-c-Met can be antagonized by anti-HGF monoclonal antibody. LNCaP-RANKL cells expressed high level of cMet at both RNA and protein levels detected by RT-PCR and western blot analyses. (D) The levels of RANKL, c-Met and p-c-MET expression in LN-RANKL and LN-Neo cells were also demonstrated by single quantum dot labeling (SQDL) of each protein with DAPI nuclear staining (E) RANKL treatment or overexpression up-regulated cMet transcriptional activity, which can be attenuated by OPG treatment. *, P<0.05; **, P<0.005. (F) Representative images of c-Met and p-c-Met IHC staining of LNCaP-RANKL-induced bone and adrenal gland tumors.

FIG. 16 shows that the RANKL overexpressing and mesenchymal cell-like LNCaP cells harbor drastic tumorigenic and metastatic potential in accordance with various embodiments of the present invention. 3D μCT scans were used to detect bone tumor formation (arrow). Whereas LN-neo control cells (the scan on left) did not present any tumor formation, LN-RANKL cells inoculated intracardially to male athymic mice caused high incidence of tumor formation both in bone (the scans on right) and in soft tissues (Table 1).

FIG. 17 shows RANKL promoter (2.5 Kb) activity in human osteosarcoma and prostate cancer cells in accordance with various embodiments of the present invention. (A) The 2.5 Kb RANKL promoter was introduced into human osteosarcoma SaOS-2 cells to examine for RANKL basal promoter activity as well as the stimulated promoter activity by exogenous treatments with PTH and vitamin D₃. RANKL transcriptional activity was up-regulated upon treatments with PTH and vitamin D3 in SaOS-2 cells. (B) RANKL promoter activity was examined in various prostate cancer cells of ARCaP_{E}, ARCaP_{M}, LNCaP, C4-2, and PC3 cells and compared to that in osteosarcoma SaOS-2 cells as a positive control (*, p<0.05). RANKL promoter reporter activity correlates with the expression level of RANKL as well as the aggressiveness of the prostate cancer cells (FIG. 17B).

FIG. 18 demonstrates that RANKL induced an autocrine feed-forward induction of RANKL expression in prostate cancer cells in accordance with various embodiments of the present invention. (A) RANKL treatment up-regulated RANKL expression at both mRNA and protein levels in ARCaP_{E} and LNCaP cells, which can be attenuated upon addition of OPG. RANKL expression in ARCaP_{M} cells can be down-regulated by addition of 1 ug/ml of OPG. (B) Corresponding results were also observed in using RANKL promoter reporter assay for which RANKL promoter activity was induced by RANKL treatments in ARCaP_{E} and ARCaP_{M} cells. OPG treatment could dampen the RANKL promoter activity in ARCaP_{M} cells and abolish the RANKL autocrine induction. (C) RANKL autocrine induction was also observed in LNCaP cells demonstrated by RANKL promoter reporter assay. RANKL promoter activity was significantly up-regulated in (D) LNCaP-RANKL and (E) ARCaP_{E}-RANKL cells compared to that of Neo control of both cell types, and OPG treatment attenuated the increased RANKL promoter activity in both LNCaP-RANKL and ARCaP_{E}-RANKL cells (*, p<0.05; **, p<0.005).

FIG. 19 demonstrates the identification of cMyc binding motif within the −1884 and −1384 region of RANKL promoter that mediates the RANKL autocrine feed-forward induction by RANKL in accordance with various embodiments of the present invention. (A) Diagram of RANKL full promoter construct and deletion mutants, D1 deleted from −2383 to −1884, D2 deleted from −1884 to −1384, D3 deleted from −1384 to −884, and D4 deleted from −884 to −384, and D5 deleted from −384 to −101. (B) The transcriptional activities of these RANKL promoter deletion mutants were examined in LNCaP-RANKL and LNCaP-Neo cells. D2 mutant showed a significant reduction of RANKL transcriptional activity to the lowest level in LNCaP-RANKL cells, and all deletion mutants showed minimal responsiveness in LNCaP-Neo cells. (C) The DNA sequence of D2 region (500 bp) within the RANKL promoter (−1884˜−1384). CRE (−1177˜−1184) and cMyc/Max (−1372˜−1384) binding sites were identified within the D2 region (underlined). (D) The full length RANKL promoter and D2, CRE (black), and cMyc(white) deletion mutants were examined for their RANKL transcriptional activity in LNCaP-RANKL and LNCaP-Neo cells. The RANKL transcriptional activity was significantly inhibited with the cMyc deletion mutant in LNCaP-RANKL cells. (**, p<0.005).

FIG. 20 shows that RANKL autocrine activation is mediated through direct interaction of cMyc with its cis-acting binding element within the RANKL promoter region in prostate cancer cells in accordance with various embodiments of the present invention. (A) Semi-quantitative RT-PCR of ChIP analysis shows increased cMyc PCR products in RANKL-treated and LNCaP-RANKL cells. This implicates that the cMyc in RANKL-treated or LNCaP-RANKL cells is transcriptionally activated by directly binding to its corresponding cis-element within the RANKL promoter, leading to the activation of RANKL transcription. Exposure to OPG can attenuate the interaction between cMyc and its binding motif within RANKL promoter, indicated by the reduced PCR products of cMyc. (B) Using the quantitative real-time PCR, the inventors observed an approximately 20 fold and 11 fold increase of cMyc binding to the cMyc site in LNCaP-RANKL and RANKL-treated LNCaP cells, respectively, and such induction can be dampened by exposing to OPG. (C) EMSA analysis demonstrating nuclear cMyc/Max heterodimer binding to cMyc oligonucleotides in RANKL-treated LNCaP and LNCaP-RANKL cells in vitro. Nuclear extract from both cells was incubated with biotin-labeled cMyc/Max probe (lanes 2-6). An excess of unlabeled probe (400×) as competitor (lane 3), anti-cMyc antibody (lane 4), anti-Max antibody (lane 5), and anti-rabbit IgG as negative control (lane 6) was added to the binding reaction. Arrows indicated free probe, cMyc/Max-DNA complex, and supershifted cMyc/Max-DNA complex conjugated with anti-cMyc or anti-Max antibodies. (D) The inventors also examined the nuclear protein levels of cMyc and Max (heterodimer of cMyc) in LNCaP cells treated with RANKL and LNCaP-RANKL cells by Western blot analysis, and lamin A/C was used as the internal control. The nuclear levels of cMyc and Max were higher in RANKL-treated LNCaP and LNCaP-RANKL cells compared to that of LNCaP cells. RANKL promoter reporter activity and protein expression level was also examined in RANKL-treated LNCaP and LNCaP-RANKL cells in the presence of 20 μM of cMyc inhibitor, 18005-F4, the cMyc inhibitor significantly decreased the RANKL transcriptional activity in LNCaP-RANKL cells or in LNCaP cells treated with RANKL. E and F, RANKL treated LNCaP cells and LNCaP-RANKL cells were treated with 20 μM of cMyc inhibitor, 10058-F4 and examined for the RANKL promoter reporter activity as well as protein expression level. cMyc inhibitor significantly decreased RANKL transcriptional activity (E) and protein expression (F) in LNCaP-RANKL cells or in LNCaP cells treated with RANKL. *, p<0.05; **, p<0.005.

FIG. 21 depicts autocrine and paracrine effects of the RANKL overexpression in accordance with various embodiments of the present invention. RANKL overexpressing PCa cells were examined for autocrine and paracrine functions with multiple expression profiling methods. (A) representative results with RayBio antibody arrays indicate altered soluble factor production in LN-RANKL culture medium, circles indicating a unique MCP-2 induction upon co-culture with the parental LN cells which expressed low to absent levels of RANKL. (B) the induction is quantified. Asterisks indicate statistical significance (P<0.01). A selected list of altered soluble factor production is shown in Table 2, where expressions being confirmed at the protein level are shown in black, while differential expressions at the mRNA level are underlined. These data support the concept that in the presence of RANKL positive LNCaP cells, RANKL negative LNCaP cells produced soluble factors that could be responsible for driving osteoblastic reactions of PCa in mouse skeleton.

FIG. 22 shows that SREBP-1, a gene found to be overexpressed in LN-RANKL cells, promotes fatty acid synthesis. LN-SR-1 and LN-SR-2 represent LN clones stably expressing high levels of SREBP-1 protein (precursor, 125 kDa; mature, 68 kDa) in accordance with various embodiments of the present invention. A, the clones were found to express elevated levels of fatty acid synthase (FAS), Nox5 and decreased catalase proteins which affect the oxidative status of prostate cancer cells, as well as increased AR (androgen receptor) protein and enhanced PI3K-Akt activity. B, these cells were found to accumulate lipid droplets and H₂O₂, with statistically significant differences between LN-SR and LN-Neo cells (p<0.005). These results, taken together, further extended the RANKL/RANK/OPG/c-Met relationship to metabolic cascade of prostate cancer cells, specifically relevant to lipid metabolism, production of reactive oxygen species (ROS), and cell growth and differentiation programs regulated by AR and PI3K-Akt pathway, controlled downstream by RANKL in human prostate cancer cells.

FIG. 23 shows that LN-RANKL cells promote maturation of pre-osteoclasts in accordance with various embodiments of the present invention. LN-RANKL cells were co-cultured with the mouse pre-osteoclast Raw264.7 cells for 7 days. Multinucleated mature osteoclasts were detected by TRAP staining Monoculture of Raw264.7 with RANKL protein addition (100 ng/ml) was used as positive control. In addition, OPG protein was added to the co-cultures to block specifically the RANKL function. Representative stains and total numbers of TRAP multinucleated cells are shown. Asterisks indicate statistical significance compared to those of the LN-RANKL cells (P<0.01).

FIG. 24 shows that LN-RANKL cells facilitate the tumor formation and metastasis of non-tumorigenic and non-metastatic LN^{RFP} cells in accordance with various embodiments of the present invention. In this study, the non-tumorigenic and non-metastatic LN^{RFP} cells were co-inoculated with LN-RANKL cells (at a 9:1 ratio) to athymic mice via intracardial route. The subjects were kept for 2 months for tumor formation. (A) a comparative ex vivo imaging shows that mouse inoculated with LN^{RFP} cells alone did not form xenograft tumors (Controls on left), while in the mouse subjected to co-inoculation, both bone and lymph node metastases now contain red fluorescent cells. (B) μCT and X-ray imaging show a representative mixed osteoclastic and osteoblastic tumor in a tibia, with arrows indicating the osteoblastic new bone formation. (C) bone and lymph node metastases shown in A were detected ex vivo for the presence of red fluorescent LN^{RFP} cells in metastatic bone and lymph node metastases and also found in bone cells derived from ex vivo culture.

FIG. 25 depicts autocrine activation of specific signaling pathways by RANKL overexpression in accordance with various embodiments of the present invention. In this study, LN-RANKL cells were examined by Western blotting for constitutive activation of conventional signal transduction pathways. Among many pathways, consistently elevated phosphorylation of PI3K/Akt, P38 and NFκB proteins was found. The results have been confirmed by alternative studies, in which the parental LN cells were treated with RANKL protein before detection of the elevated phosphorylations.

FIG. 26 shows that RANKL increased the tumorigenic potential of both LNCaP and ARCaP cells by inducing increased anchorage-independent colony formation compared to the LNCaP-Neo and ARCaP_{E}-Neo cells, and such induction can be attenuated upon addition of OPG in accordance with various embodiments of the present invention.

FIG. 27 depicts RANKL expression by gene transfer in non-metastatic human cancer cells in accordance with various embodiments of the present invention.

FIG. 28 depicts that RANKL expression leads to enhanced RANKL and c-Met expression/phosphorylation in prostate cancer cells in accordance with various embodiments of the present invention.

FIG. 29 depicts that RANKL-expressing but not neo-control human cancer cells metastasized to mouse skeleton in accordance with various embodiments of the present invention.

FIG. 30 depicts increased TRAP+ osteoclasts in mouse skeleton harbored with metastatic human cancer cells in accordance with various embodiments of the present invention.

FIG. 31 depicts that RANKL significantly increased the ability of human cancer cells to metastasize to bone and soft tissues in accordance with various embodiments of the present invention.

FIG. 32 depicts that as few as 1,000 RANKL-expressing prostate cancer cells are capable of inducing cancer bone colonization of non-tumorigenic and non-metastatic prostate cancer cells in accordance with various embodiments of the present invention.

FIG. 33 depicts that RANKL-expressing human prostate cancer cells induced RANKL and c-Met expression/phosphorylation in non-metastatic prostate cancer cells in accordance with various embodiments of the present invention.

FIG. 34 depicts that recombinant sRANKL promotes bone colonization by human cancer cells in accordance with various embodiments of the present invention.

FIG. 35 depicts that knockdown of RANK receptor depressed prostate cancer bone colonization in accordance with various embodiments of the present invention.

FIG. 36A and FIG. 36B depict that C-Myc-Max serves as a common transcription factor complex, binds to the cis-element (E-box) resides in human RANKL and c-Met promoters, and activated promoter-Luc reporter activities in accordance with various embodiments of the present invention.

FIG. 37 depicts that RT-PCR results demonstrate the induction of stem cell and neuroendocrine cell phenotypes In LNCaP cells overexpressing RANKL in accordance with various embodiments of the present invention.

FIG. 38 depicts that A RANK decoy receptor, OPG, and a recombinant RANKL Ab, denosumab, antagonized the expression of stem cell markers by RANKL-expressing prostate cancer cells in accordance with various embodiments of the present invention.

FIG. 39 depicts that increased expression of other stem cell markers, CD44, CD49f, and OCT3/4, by RANKL expression in prostate cancer cells as analyzed by FACS in accordance with various embodiments of the present invention.

FIG. 40 depicts that OPG and denosumab also blocked the expression of neuroendocrine markers by RANKL-expressing prostate cancer cells in accordance with various embodiments of the present invention.

FIG. 41 depicts that RANKL expression by prostate cancer tissues predicts the survival of prostate cancer patients in accordance with various embodiments of the present invention.

FIG. 42 depicts that RANKL-expressing tumor growth can be most effectively blocked by the co-administration of denosumab and XL-184 in accordance with various embodiments of the present invention.

FIG. 43 depicts model of RANKL-RANK signaling and cancer bone metastasis in accordance with various embodiments of the present invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

“Cancer” and “cancerous” refer to or describe the physiological condition in mammals that is typically characterized by unregulated cell growth. Examples of cancer include, but are not limited to, breast cancer, colon cancer, lung cancer, prostate cancer (including but not limited to prostate cancer, castration resistant prostate cancer, androgen-independent prostate cancer, androgen-dependent prostate cancer), hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer (including, but not limited to, gliomas, glioblastomas, glioblastoma multiforme (GBM), oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary adenomas, neuroblastomas, and craniopharyngiomas).

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus adult and newborn subjects, as well as fetuses, whether male or female, are intended to be including within the scope of this term.

“Therapeutically effective amount” as used herein refers to that amount which is capable of achieving beneficial results in a patient; for example, a patient with cancer. A therapeutically effective amount can be determined on an individual basis and will be based, at least in part, on consideration of the physiological characteristics of the mammal, the type of delivery system or therapeutic technique used and the time of administration relative to the progression of the disease.

The current invention describes a method to test prostate cancer cell derived RANKL in the pathogenesis and metastasis of human prostate cancer cells with results confirmed in clinical human prostate cancer specimens. Using both ARCaP and LNCaP models, the inventors demonstrated the critical transition of epithelium-like prostate cancer cells to mesenchymal phenotype (EMT) and the propensity of prostate cancer cell to gain bone and soft tissue metastases. The transition of epithelium-like prostate cancer cells to their mesenchymal-like cells can be provoked by soluble growth factors or cellular interaction with host bone or adrenal gland, which ultimately increased cell growth, migration, and invasion in vitro. For example, ARCaP_{M} derived from ARCaP_{E} through EMT trans-differentiation show increased invasive and migratory potential in vitro as well as the ability to metastasize to bone and soft tissues in mice (Xu et al., 2006). Likewise, EMT trans-differentiation can occur in LNCaP cells at the biochemical level (but without evidence of morphologic transition) with these cells gained increased mesenchymal (such as increased expression of vimentin, but decreased expression of E-cadherin) and cell behavioral (such as increased cell migration and invasion) phenotypes capable of metastasizing to bone and soft tissues. The present invention uses both of these cell models with results confirmed in clinical metastatic human prostate cancer specimens. As an example, ARCaP_{E} and ARCaPₘ cells render a cell environment for the study of RANKL/RANK/OPG system on the regulation of osteoclastogenesis and osteoblastogenesis in bone metastasis. Moreover, it is interesting and important to identify the functional role of RANKL in human prostate cancer cells other than osteoclastogenesis induced by prostate cancer. As described herein, the inventors characterized its role by knocking down and overexpressing RANKL in ARCaP and LNCaP models, and the inventors looked into the behavioral changes of these cells and the relationship to the transcriptional regulation of RANKL, c-Met and androgen receptor (AR) promoters.

Understanding the biology and targeting of prostate cancer bone metastasis holds promise for improved survival of patients with castration-resistant and lethal progressive disease. Among many biologic processes studied, the inventors and others found the induction of EMT by soluble growth factors can provoke increased cancer cell growth, invasion, migration and ultimately metastasis to distant organs. Previous elegant studies demonstrating paracrine roles of tumor-derived PTHrP which promotes RANKL expression by osteoblasts, increasing bone turnover through a “vicious cycle”, and linked cellular interactions among cancer (which expresses PTHrP), osteoblasts (which express RANKL) and osteoclasts (which express RANK). Potential paracrine roles of RANKL in mediating hormone-induced stromal-epithelial interaction in mammary gland development, and expansion of “stem cell niche” providing an escape for cancer cells to gain altered behaviors such as increased EMT and resistance to hormone-, chemo- and/or radiation-therapy have also been proposed. The present study added additional understanding the roles of tumor cell-derived RANKL in EMT, and further defined the converging RANKL-c-Met forward feedback loop to explain the roles of prostate cancer cell-derived RANKL in bone and soft tissue metastases. The significance of these observations are: 1) RANKL derived from tumor cells or its microenvironment could amplify RANKL downstream growth and survival signaling by promoting RANKL and c-Met expression. Both RANKL and HGF have been shown to induce EMT in prostate cancer cells and activation of downstream signaling network involving c-Met which is activated primarily by VEGF in ARCaP cell model through its co-receptor, neuropilin-1, to promote survival by activating an anti-apoptotic gene, Mcl-1; 2) The expressed RANKL and c-Met in cancer cells are biologically functional to participate in an enhanced osteoclastogenesis and through increased c-Met phosphorylation and downstream survival signaling; 3) A small number of RANKL expressing prostate cancer cells are sufficient to facilitate the growth and colonization of RANKL-null cells in mouse skeleton suggesting RANKL can serve as a factor in “reawakening” cancer dormancy in mouse bone. Based on trafficking of LNCaP-RFP cells, the inventors observed prostate cancer bone metastasis contributed directly to its secondary soft tissue metastases; 4) These findings support the significant clinical insights gained from targeting of RANKL and c-Met/VEGF based signaling by an anti-RANKL monoclonal antibody, denosumab, and a small molecule cabozantinib (XL-184) which were shown to delay or prevent the progression of castration-resistant prostate cancer (CRPC) in patients with bone metastasis.

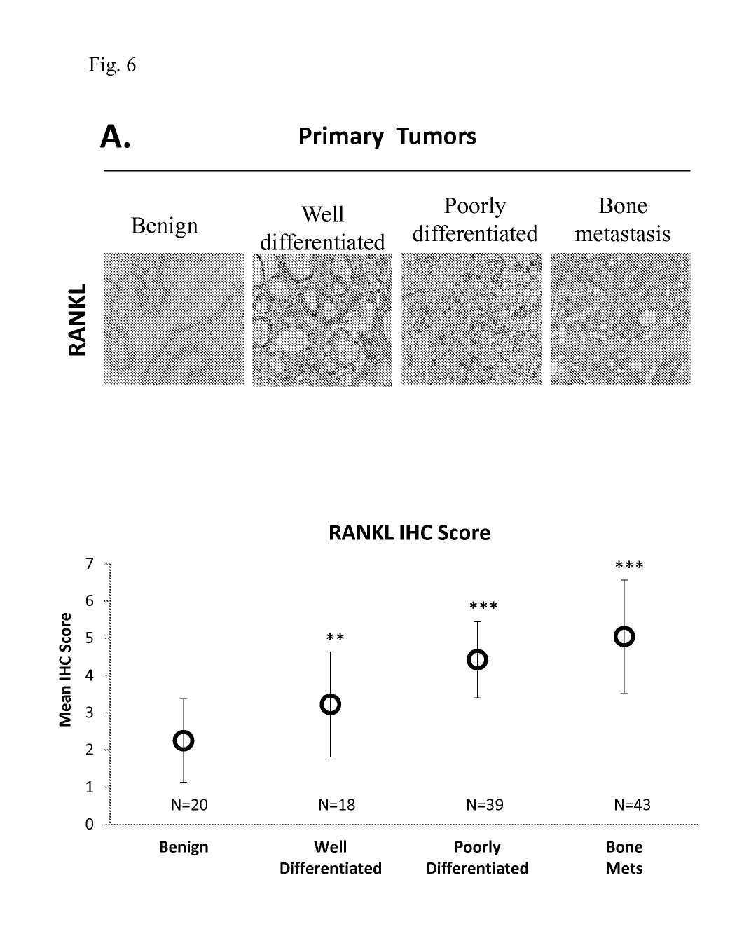

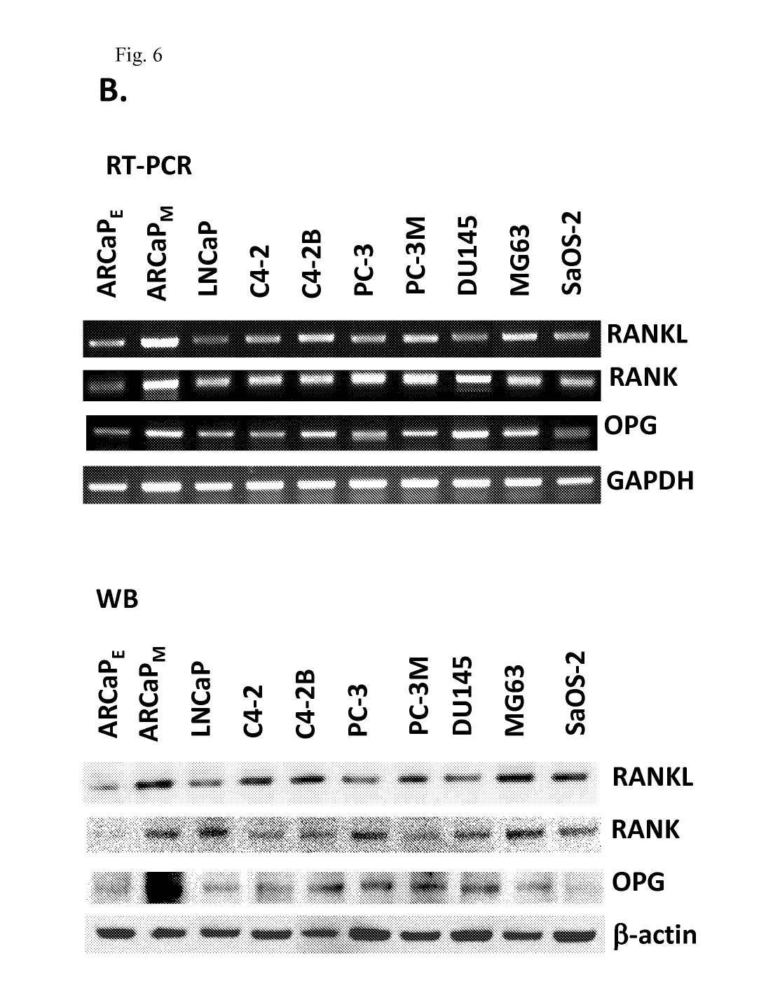

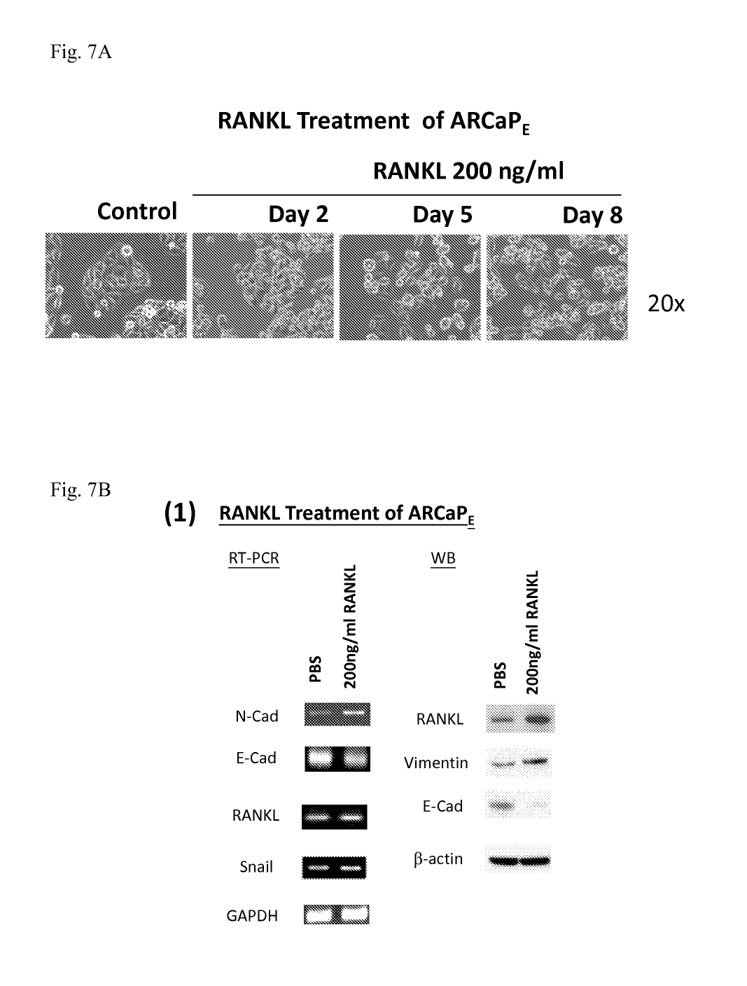

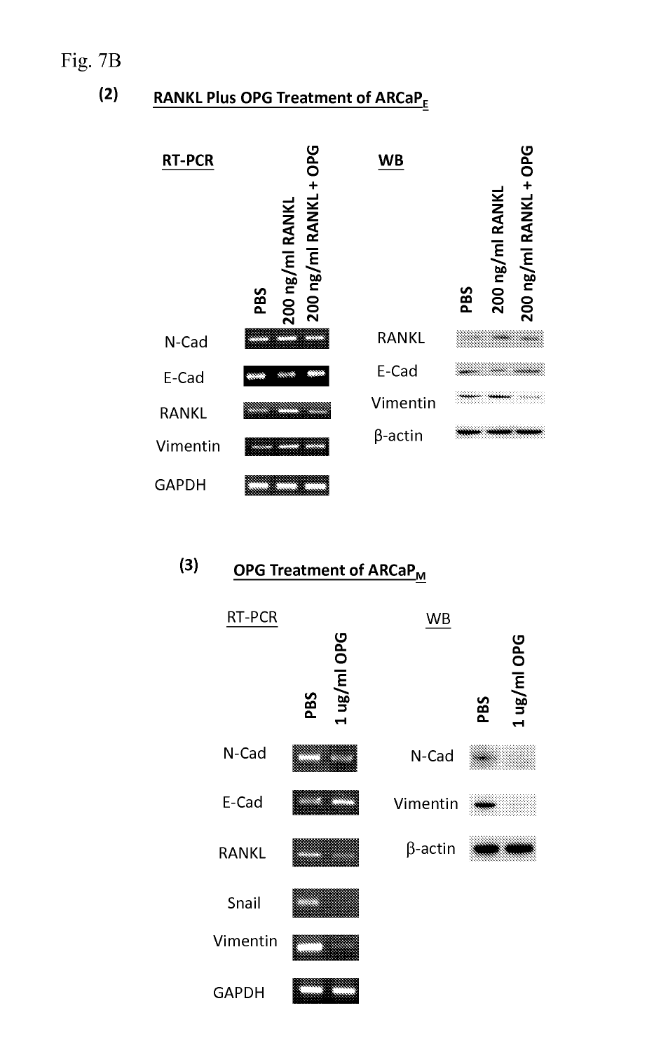

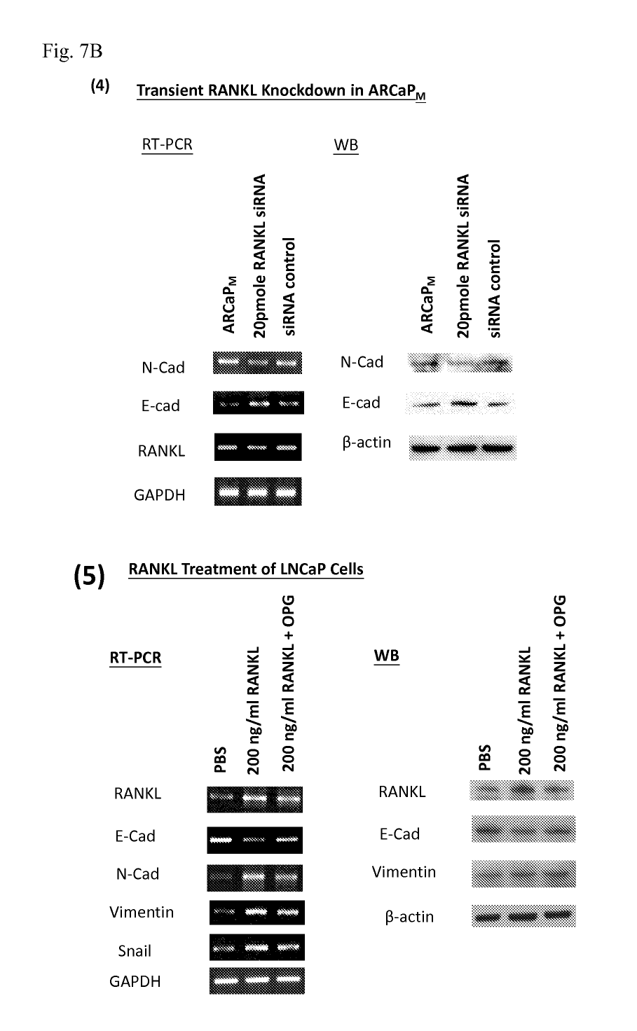

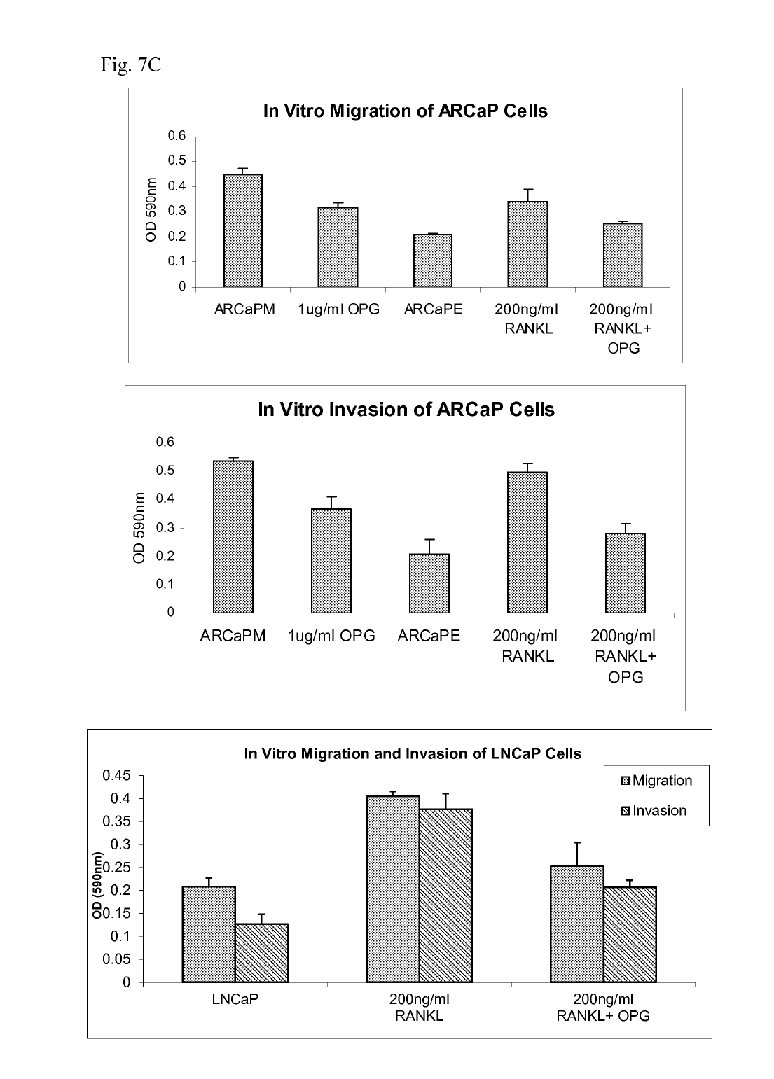

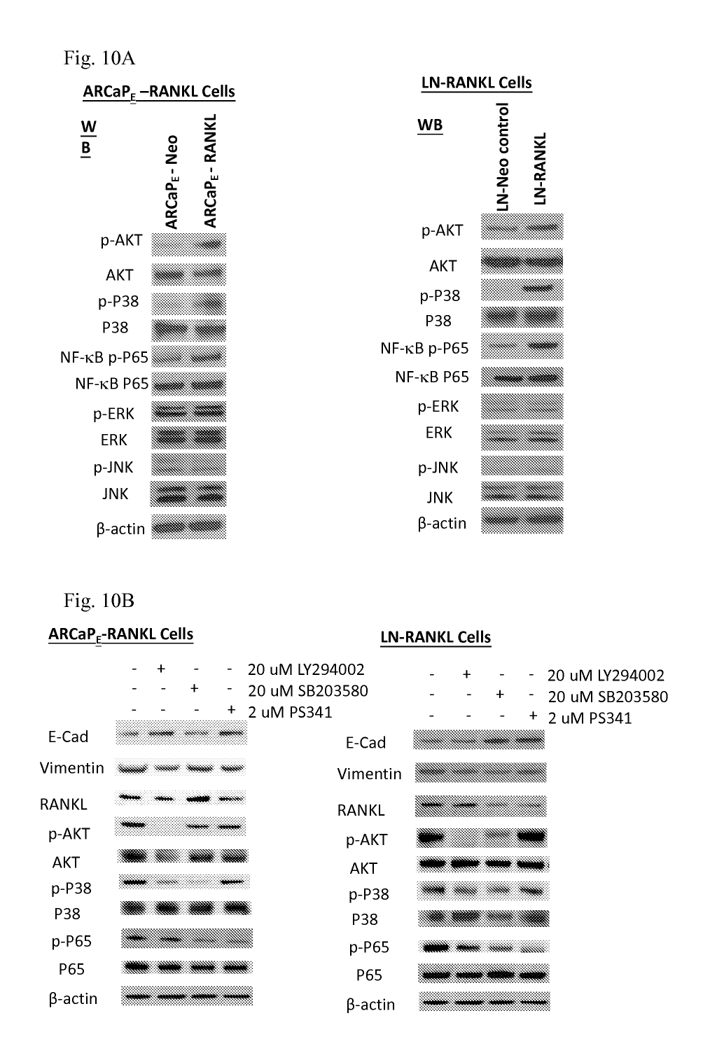

The functional roles of RANKL derived from prostate cancer and its contribution to bone and soft tissue metastases have not been investigated previously in part due to the lack of evidence of a predominant expression of RANKL by prostate cancer cells which were found to colonize the bone. The inventors' results of RANKL amplifying RANKL and c-Met expression and the facilitating roles of a small number of RANKL-expressing prostate cancer cells to promote RANKL-null cells co-colonize bone reaffirmed the potential dynamic role of RANKL in prostate cancer bone and secondary soft tissue metastases. The evidence is further supported by the fact that: 1) RANKL, RANK and OPG are expressed by clinical human prostate cancer tissues and also by a wide-spectrum of isogenic human prostate cancer cell lines (FIGS. 6A and 6B). The steady-state level of RANKL expression in these cell lines seems to correlate with increased invasiveness and bone metastatic potential of prostate cancer tissues and cells (FIG. 6B). 2) Interrupting RANKL-RANK interaction with bisphosphonate or denosumab in men with clinical progressive prostate or breast cancers reduced their skeletal related events and improved patients' survival. 3) Using human prostate, breast, lung and renal cancer cell lines as experimental models for the study of underlying mechanism driving cancer bone metastasis, the inventors found a consistent elevation of RANKL, that was provoked by soluble factors, which drive EMT and cancer bone and soft tissue metastases and induced lethality in experimental mice. 4) Previous studies have focused on the sources of RANKL from normal cells, such as osteoblasts B- or T-cells, which often activate their receptor RANK in the neighboring normal cells in a paracrine manner. Results of this study support a model in which prostate cancer cell-derived RANKL plays a pivotal role in conferring the ability of tumor cells to metastasize to bone through an induction of EMT driven by PI3K-Akt, P38-MAPK and NF-κB signaling (FIG. 10A). This model, however, does not exclude the important functional roles of paracrine RANKL-RANK interaction. The positive-feedback loop of RANKL regulation of its own expression in prostate and bone cancer but not normal cells (FIG. 7B, 8A, 8C), raising the possibility of a “vicious cycle” in cancer but not normal cells in which autocrine RANKL-RANK signaling is amplified in prostate cancer bone metastasis. RANKL-RANK signaling, however, also occurs with paracrine interaction between osteoblasts and osteoclasts during bone remodeling. RANKL also serves as a paracrine mediator in steroid hormone action in mammary gland development and stem cell renewal during pregnancy, and is also involved in lymph node organogenesis, monocyte function and inflammatory responses.

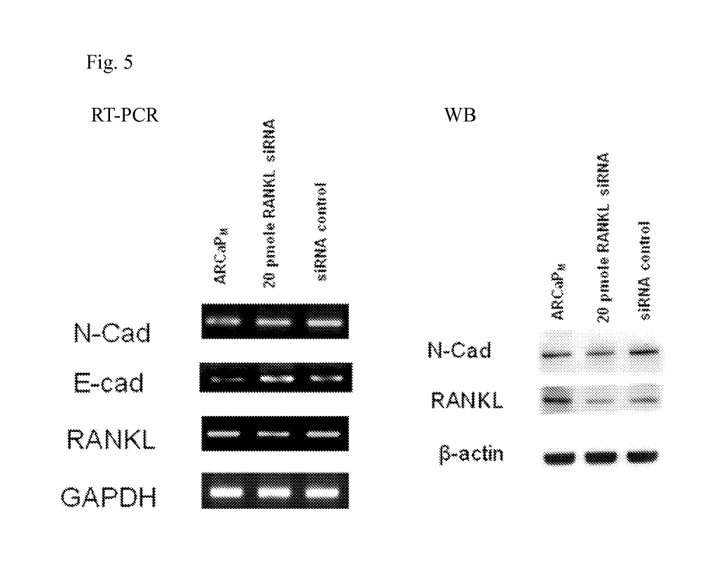

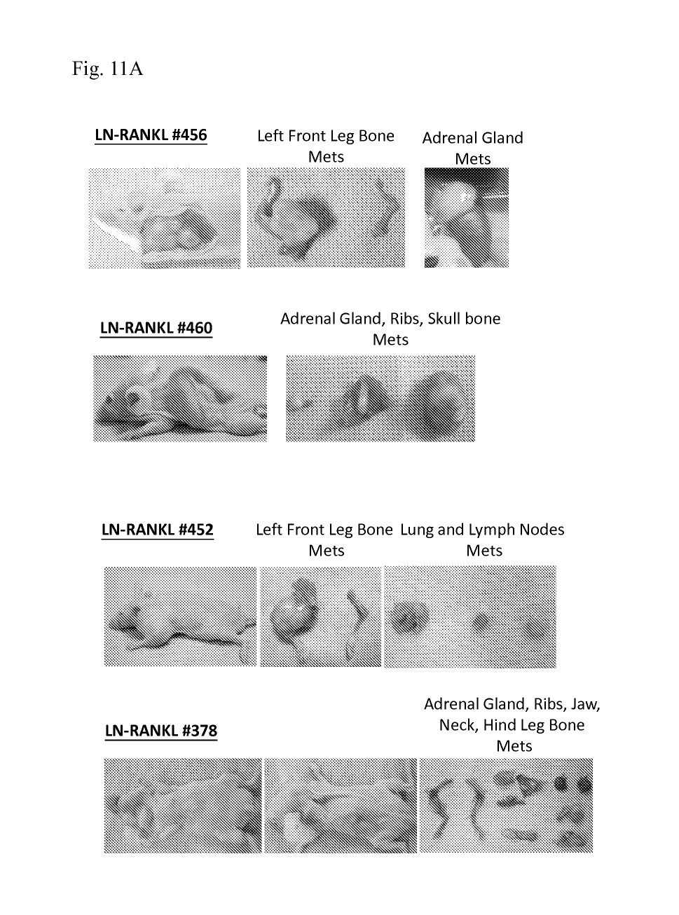

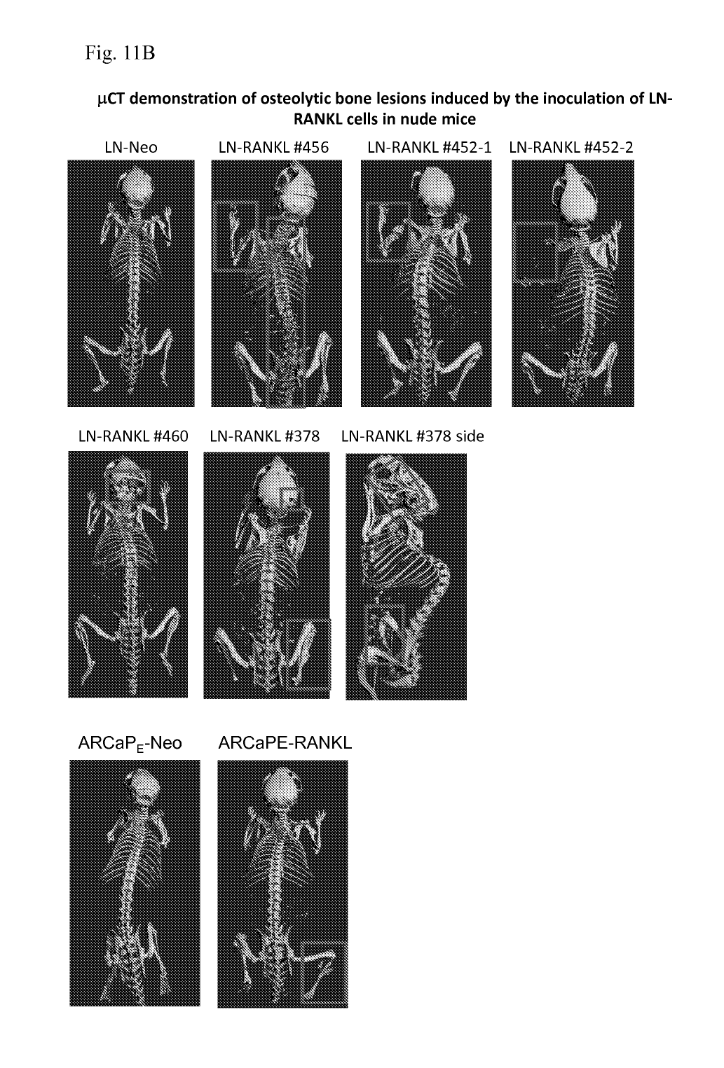

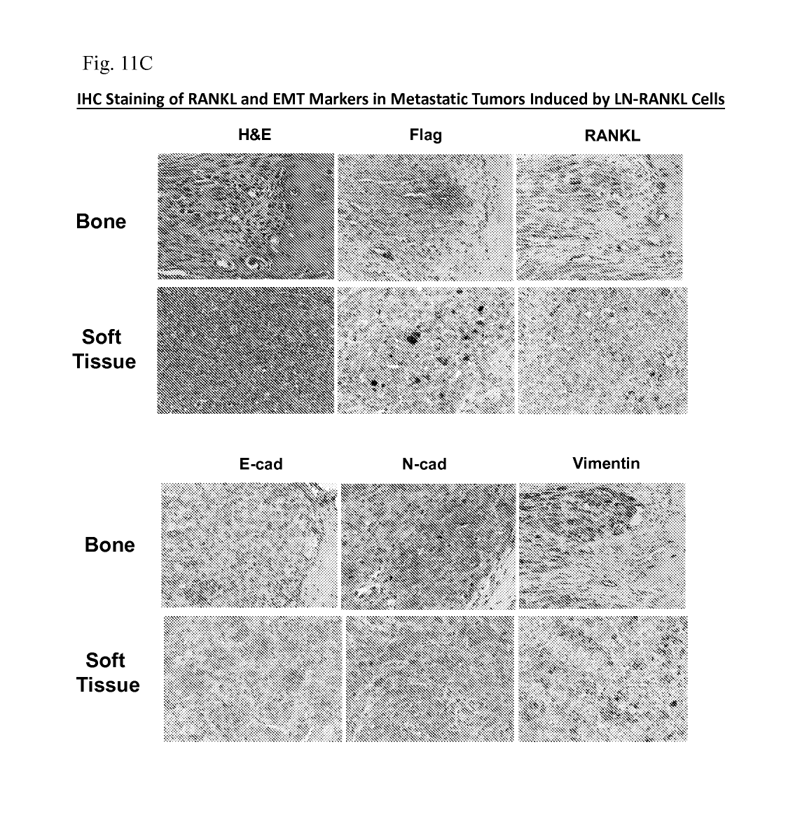

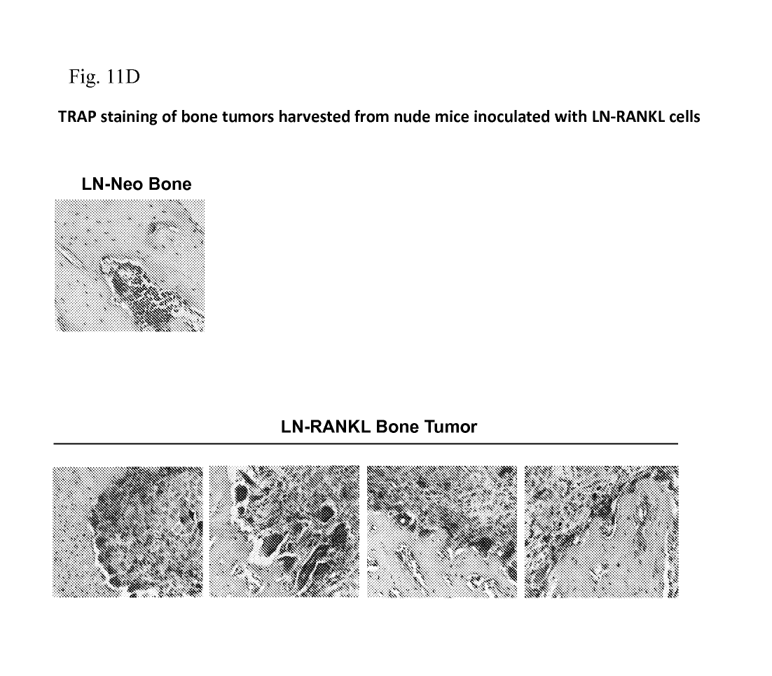

Autocrine RANKL-RANK interaction within prostate cancer cells was shown to modulate “cadherin switch” which controls EMT and its reversal MET. In reality, however, in human prostate cancer bone metastasis, EMT is considered as an important early invasive step in metastasis but once tumor cells reached distant organs a reversal of EMT, or MET occurs. The data herein obtained from the ARCaP EMT model support this suggestion since antagonizing RANKL-RANK interaction genetically by RANKL siRNA (FIG. 7B-4) or blocking their downstream signaling by pathway-specific inhibitors (FIG. 10B), the inventors have observed a reversal of EMT, or MET. The reversal could offer an explanation of why the bulk of prostate cancer cells in circulating blood and in bone appear epithelial in morphology and express E-cadherin and EpCAM. The understanding of RANKL-RANK switch in EMT and its reversal to MET could offer one explanation for the mechanism. Since clinical prostate cancer bone metastasis is predominately osteoblastic, yet the animal models revealed primarily osteolytic bone reactions (FIG. 11D), while not wishing to be bound by any particular theory, the inventors believe that factors yet to be identified within the tumor microenvironment could play a pivotal role in attenuating autocrine RANKL-RANK signaling in prostate cancer cells. For example, elevated OPG or decreased osteoclast activating factors such as IL-11, MIP-1α, and secreted factors that control the shedding of activated RANKL by cathepsins and MMPs could dampen RANKL-RANK signaling. Attenuating RANKL expression by downregulating osteotropic growth factors, such as TGF-β, PTHrP, and prostaglandin E2 could play a role. In addition, factors secreted by bone marrow stromal cells, such as IL-4, IL-13, Wnt/β-catenin, BMP-2, and TGF-β could attenuate RANKL-RANK signal and/or activate osteoblastic activity. These factors secreted in the tumor microenvironment could be responsible for fine-tuning of RANKL-RANK signaling which ultimately will determine the cadherin and EMT/MET switches, cancer cell growth, survival and therapeutic responsiveness. Since stem cell properties have been observed in cancer cells undergoing EMT and that RANKL-RANK signaling could be responsible for the expansion of the stem cell niche, an appropriate balance of RANKL-RANK signaling could have profound implications in determining the status of malignancy of cancer cells. Additionally, RANKL-RANK interaction and downstream signaling could also determine the ability of cancer bone colonization and the coupling between osteolytic versus osteoblastic responses observed in clinical prostate cancer bone metastasis. Consistent with other studies, activation of RANK can enhance cell migration and invasion of prostate and non-prostate cancer epithelial cells.

Prostate cancer cells have been shown to exhibit osteomimetic properties, allowing them to imitate gene expression and the function of bone cells. The inventors found that one of the factors controlling prostate cancer cells' synthesis and deposition of osteocalcin and bone sialoprotein is β2-microglobulin. Interestingly, β2-microglobulin expression in human prostate, breast, lung and renal cancers increased RANKL expression and promoted EMT and cancer skeletal and soft tissue metastases resulting in increased lethality in mice. The inventors speculated that RANKL expression by these cancer cells might be responsible, in an autocrine manner, the morphologic, biochemical and behavioral transition of a prostate cancer cells to express their migratory, invasive and metastatic behaviors through EMT. However, since RANKL expressed by prostate cancer cells is functional in the induction of osteoclastogenesis in vitro, this suggests RANKL-RANK interaction in prostate cancer cells must be involved also in the host microenvironment. The inventors examined the requirement of three RANKL-RANK downstream signaling network, PI3K-Akt, P38-MAPK, and NF-κB, in EMT by the use of appropriate pathway-specific metabolic inhibitors. Results indicate that RANKL-induced EMT was abrogated by inhibiting NF-κB signaling in both ARCaP_{E}-RANKL and LNCaP-RANKL cell models; however, inhibition of P38-MAPK or PI3K-Akt also partially reverted the EMT marker and decreased RANKL expression in both ARCaP_{E}-RANKL and LNCaP-RANKL cells (FIG. 10B). The inventors observed significant pathway “cross-talk” occurs since both P38 and PI3K inhibitors diminished the level of P65 phosphorylation, and that P38 inhibitor had a stronger inhibitory effect on NF-κB activation than that of PI3K inhibitor (FIG. 10B). Additionally, PI3K inhibitor significantly reduced the phosphorylated level of P38 in both cells (FIG. 10B). These results are therefore in agreement with several studies, which have shown that activation of P38 MAPK is required for P65 phosphorylation and transcription function, and Akt can transactivate P65 subunit of NF-κB through the activation of P38-MAPK. Furthermore, studies using malignant melanoma also showed that the ERK pathway is not involved in NF-κB activation. These results suggested that RANKL induced a sequential activation of signaling cascades from PI3K-Akt, P38, and then to NF-κB in ARCaP_{E}-RANKL and LNCaP-RANKL cells. Therefore, RANKL mediates EMT transformation of prostate cancer cells by transactivating NF-κB signaling through an Akt and P38 dependent pathways, which are known to be involved in cancer cell proliferation, survival, and conferring cancer distant metastasis.

The inventors' study identifies an important role for prostate cancer cell derived RANKL in prostate cancer bone and soft tissue metastases through the induction of EMT. The inventors observed an intriguing RANKL autocrine signal amplification system in which RANKL induced its own expression in cancer but not normal cells. The inventors showed RANKL-RANK interaction activated downstream PI3K-Akt, P38-MAPK, and NF-κB. The inventors suggest fine-tuning the RANKL-RANK switch in prostate cancer cells could elicit insights in the biology, such as EMT, osteoblastic versus osteolytic lesions induced by metastatic prostate cancer, and improve therapeutic targeting of RANKL-RANK axis. Understanding the roles of tumor cell-derived RANKL in EMT and tumor dormancy could strengthen the rationale of targeting RANKL-c-Met-mediated downstream signaling and improve the effectiveness of targeting lethal bone metastasis of CRPC.

ARCaP_{M} cell lines are highly metastatic prostate cancer models, which are the best candidates to be used to study and evaluate the involvement of epithelial to mesenchymal transition as well as the host microenvironment in prostate cancer bone metastases. The two subclones of ARCaP cells are mesenchyme-like ARCaP_{M} and epithelium-like ARCaP_{E}. Previous results showed that the RANKL protein expression is abundantly detected in ARCaP_{M} cells, with low level of expression detected in ARCaP_{E} cells. This finding is in accordance with the highly bone metastatic nature of ARCaP_{M} cell of mesenchymal type, which is derived from epithelial ARCaP_{E} through EMT transdifferentiation and the interaction of ARCaP_{E} with the host bone. Since RANKL is the key regulator for the osteoclastogenesis of bone metastasis, the expression of RANKL is expected to be up-regulated in the bone metastasizing ARCaP_{M} cells but not in the epithelial ARCaP_{E} cells. This further proves that ARCaP_{M} cells are more aggressive in invasion and migration in vitro and in metastasis to bone in mice. Therefore, using this cell model allowed the inventors to study more closely the relationship between the host microenvironment EMT and the propensity of prostate cancer to metastasize to bone and soft tissues, which offers the most suitable cell system for investigating the regulation of RANKL/RANK/OPG of the bone remodeling process. Moreover, the inventors also wanted to identify the function of RANKL in human prostate cancer cells and how its expression in the prostate cancer cells can lead to bone metastasis. Therefore, the inventors have tackled this issue from three directions: 1) RANKL treatment and genetically overexpression of RANKL in ARCaP_{E}; 2) Knockdown RANKL expression in ARCaP_{M}; and 3) use of RANKL promoter region to study both function and transcription regulation of RANKL and also to determine its role in promoting EMT in prostate cancer cells.

The inventors first treated ARCaP_{E} cells with RANKL, and the inventors demonstrated that N-cadherin increased and E-cadherin decreased, underlying the EMT process. Other mesenchymal markers also went up, such as vimentin. However, OPG can block RANKL, thus making the cell more epithelial-like again. One thing to note is that RANKL treatment decreased E-cadherin expression much more dramatically in protein level compared to the RNA level. This might imply that RANKL may be involved in the E-cadherin translational regulation or protein degradation but not so much at the RNA regulation. Similar effects have seen in overexpression of RANKL in ARCaP_{E} cells with E-cad decrease and N-cad and vimentin increase. The inventors have also transiently knocked down RANKL in ARCaP_{M} cells, and the inventors found that knocking down RANKL slightly down-regulated N-cad expression at both RNA and protein levels and up-regulated E-cad expression more obviously. From these data, it is clear that RANKL is indeed involved in the EMT process of epithelial-like ARCaP_{E} cells to become more mesenchymal-like and more aggressive. Since both ARCaP cells express both RANKL and RANK, it means that RANKL can act on RANK via an autocrine fashion. While not having to be bound by any particular theory, the inventors believed that once ARCaP_{E} cells are stimulated by growth factors or cytokines, it can significantly increase RANKL secretion, which then bind to its own RANK on the cell surface to induce EMT or cell differentiation/transformation to become more aggressive and behaving like ARCaPₘ cells.

The inventors created RANKL stable clones in three different prostate cancer cell lines, ARCaP_{E}, C4-2, and LN cells. Highly expressed clones were selected and animal studies for the metastasis ability of these clones and for RANKL-expressing C4-2 and LN cells were performed. The inventors converted originally osteolytic metastasis to osteoblastic lesions in the bone by decreasing the ratios of RANKL-expressing LNCaP cells. The inventors can also use these clones to study the invasive properties of these cells to further prove the EMT transition by doing migration and invasion assays. During selections, the inventors have already observed some morphological changes from epithelial-like to mesenchymal-like for ARCaP_{E} cells and for C4-2 cells, some clones are known to undergo neuroendocrine differentiation, a known aggressive form of prostate cancer in patients. For promoter study, the inventors first examined the basic activity of RANKL promoter among different prostate cancer cell lines and the inventors will stimulate the promoter activity using RANKL activating factors, such as vitamin D₃ and PTH to study the regulation of RANKL promoter. Results of these studies demonstrated consistently vitamin D₃ and PTH induced RANKL promoter activities in SaOS-2 (a human osteosarcoma cell line), ARCaP_{E} and LNCaP cells.

Therefore, embodiments of the present invention are based, at least in part, on these findings described herein.

Various embodiments of the present invention provide for methods of treating cancer in a subject in need thereof.

In various embodiments, the method comprises providing a composition comprising an agent capable of inhibiting RANK and/or RANKL, and an agent capable of inhibiting HGF-c-Met/VEGFR2/neuropilin-1-mediated signaling; and administering the composition to the mammalian subject to treat cancer.

In various embodiments, the method comprises: providing a first composition comprising an agent capable of inhibiting RANK and/or RANKL, and a second composition comprising an agent capable of inhibiting HGF-c-Met/VEGFR2/neuropilin-1-mediated signaling; and administering the first and second composition to the mammalian subject to treat cancer.

In various embodiments, the agent capable of inhibiting RANK and/or RANKL is denosumab (available from Amgen). Denosumab is a fully human monoclonal antibody that specifically targets RANKL which is a key mediator of osteoclast formation, function, and survival. By targeting RANKL, downstream c-Met survival signaling will also be blocked because RANKL not only activate RANKL but also c-Met expression.

In various embodiments the agent capable of inhibiting RANK and/or RANKL is RANK-Fc, OPG-Fc for blocking RANKL, shRNA, or siRNA. In various embodiments, the shRNA or the siRNA inhibits RANKL expression.

In various embodiments, the agent capable of inhibiting HGF-c-Met/VEGFR2/neuropilin-1-mediated signaling is XL-184 (available from Exelixis, Inc.). XL184 is a small molecule designed to inhibit multiple receptor tyrosine kinases, specifically MET and VEGFR2. MET is a receptor tyrosine kinase that plays key roles in cellular proliferation, migration, and invasion as well as angiogenesis.

In various embodiments, the agent capable of inhibiting HGF-c-Met/VEGFR2/neuropilin-1-mediated signaling is cMet inhibitor PF-2341066 (Crizotinib), VEGFR2 Kinase inhibitor III (CAS 204005-46-9), denosumab, RANK-Fc, OPG-Fc for blocking RANKL, shRNA, or siRNA.

In various embodiments, the cancer is prostate, kidney, breast, bladder, lung, ovarian, pancreatic, thyroid, liver, gastric, colon or melanoma cancer. In various particular embodiments, the cancer is prostate cancer.

Various embodiments of the present invention provide for a method of preventing, reducing the likelihood of and/or inhibiting metastases of cancer cells in a mammalian subject in need thereof. The method comprises providing a composition comprising an agent capable of inhibiting epithelial-to-mesenchymal transition (EMT) of cancer cells and administering the composition to a mammalian subject in need thereof.

In various embodiments, the agent capable of inhibiting EMT is an agent capable of inhibiting RANKL. Inhibiting RANKL can include but is not limited to blocking RANKL and inhibiting the expression of RANKL.

In various embodiments, osteoprotegerin (OPG) is the agent capable of blocking RANKL. Thus, in various embodiments, the method for preventing, reducing the likelihood or inhibiting metastases of cancer cells in a mammalian subject in need thereof, comprises: providing a composition comprising a quantity of osteoprotegerin (OPG) in an amount effective to bind to RANKL and inhibit the formation of maturing osteoclasts; and administering the composition to the subject to prevent or inhibit metastases of the cancer cells. In various embodiments, the cancer cells are prostate, kidney, breast, bladder, lung, ovarian, pancreatic, thyroid, liver, gastric, colon or melanoma cancer cells. In various particular embodiments, the cancer cells are prostate cancer cells.

In various embodiments, blocking RANKL is achieved with a natural decoy receptor, such as OPG. In other embodiments, blocking RANKL is achieved with an anti-RANKL ab, such as denosumab. In other embodiments blocking RANKL is achieved with RANK-Fc or OPG-Fc. RANKL can also be targeted by blocking its expression upstream using inhibitors such as inhibitors of IL-6 (e.g., ocilizumab), inhibitors of EGF (e.g., Genistein, PD153035 or PD158780), TGF-β (e.g., SB-431542), and receptor kinase inhibitors. Inhibiting RANKL expression can also be achieved by genetic manipulation using RANKL shRNA. An example of an shRNA include but is not limited to CCGGCCCATAAAGTGAGTCTGTCCTCTCGAGAGGACAGACTCACTTTATGGGTTTTT (SEQ ID NO:17).

In various embodiments, the agent capable of inhibiting EMT is denosumab, RANK-Fc, OPG-Fc, siRNA, shRNA, XL-184, crizotinib, VEGFR2 kinase inhibitor III (CAS 204005-46-9) or combinations thereof.

Various embodiments of the present invention provide for a method of inhibiting a process of RANKL-mediated awakening of cancer dormancy, comprising: providing a composition comprising an agent capable of inhibiting epithelial-to-mesenchymal transition (EMT) of cancer cells; and administering a quantity of the composition to the a mammalian subject in need thereof to inhibiting the process of RANKL-mediated awakening of cancer dormancy.

In various embodiments, the agent capable of inhibiting EMT is osteoprotegerin (OPG) and binds to RANKL to inhibit the formation of osteoclasts, thereby inhibiting the process of RANKL-mediated awakening of cancer dormancy.

In various embodiments, the agent capable of inhibiting EMT is denosumab, RANK-Fc, OPG-Fc, siRNA, shRNA, XL-184, crizotinib, VEGFR2 kinase inhibitor III (CAS 204005-46-9) or combinations thereof. In various embodiments, the siRNA or the shRNA inhibits RANKL expression. In various embodiments, the cancer is prostate, kidney, breast, bladder, lung, ovarian, pancreatic, thyroid, liver, gastric, colon or melanoma cancer. In various embodiments, the cancer is prostate cancer.

Various embodiments of the present invention provide for a method of preventing or inhibiting the formation of osteoclasts in prostate cancer cells in a subject in need thereof, comprising: providing a composition comprising a quantity of OPG to the cancer cells, in an amount effective to bind to RANKL and inhibit the formation of osteoclasts; and administering the composition to the subject to prevent or inhibit the formation of maturing osteoclasts which are responsible, in part, for promoting the colonization of prostate cancer cells in the skeleton.

Various embodiments provide a composition for preventing or inhibiting metastases of cancer cells, comprising: a quantity of OPG in an amount effective to bind to RANKL and inhibit the formation of osteoclasts. Further embodiments of the present invention provide a composition for preventing the formation of osteoclasts in prostate cancer cells, comprising: a quantity of OPG in an amount effective to bind to RANKL and inhibit the formation of osteoclasts.

Various embodiments provide for a cell expressing a target selected from the group consisting of RANKL, an EMT marker, NF-kB, c-Met, VEGF, neuropilin-1, Mcl-1 and combinations thereof. In various embodiments, the cell is a cell overexpressing the target. In various embodiments, the cell is a prostate, kidney, breast, bladder, lung, ovarian, pancreatic, thyroid, liver, gastric, colon or melanoma cancer cell. In certain embodiments, cell is a prostate cancer cell. In certain embodiments, the prostate cancer cell is ARCaP_{E}, ARCaP_{M}, C4-2, LNCaP, PC3 or MCF7. In certain embodiments, the cell is LN-RANKL cell.

Various embodiments of the present invention provide for a method of identifying a compound that inhibits metastasis, comprising: providing a cell expressing a target selected from the group consisting of RANKL, an EMT marker, NF-kB, c-Met, VEGF, neuropilin-1, Mcl-1 and combinations thereof; contacting the cell with a test compound; and determining whether metastasis is inhibited in the presence of the test compound, wherein the decrease of the expression of the target is an indication that the test compound inhibits metastasis. In various embodiments, the decrease of the expression of the target, or its upstream signaling components, Src-kinase and Stat3 phosphorylation, are indications that the test compound inhibits metastasis. Alternatively, the RANKL pathway can be effectively blocked by the use of c-Met, VEGF-neuropilin-1, Src-kinase, Stat3 or Mcl-1 inhibitor and combinations thereof. In various embodiments, the EMT marker is selected from the group consisting of N-cadherin, VEGF and combinations thereof. In various embodiments, the cell is a cell overexpressing the target. In various embodiments, the cell is a prostate cancer cell. In various embodiments, the prostate cancer cell is ARCaP_{E}, ARCaP_{M}, C4-2, LNCaP, PC3 or MCF7.

Various embodiments of the present invention provide for an animal, comprising a cell expressing a target selected from the group consisting of RANKL, an EMT marker, NF-kB, c-Met, VEGF, neuropilin-1, Mcl-1 and combinations thereof. In various embodiments, the cell is an LNCaP-RANKL cell. In various embodiments, the animal is a mouse.

Various embodiments of the present invention provide for a method of identifying a compound that inhibits metastasis, comprising, providing the animal comprising a cell expressing a target selected from the group consisting of RANKL, an EMT marker, NF-kB, c-Met, VEGF, neuropilin-1, Mcl-1 and combinations thereof; contacting the animal with a test compound; and determining whether metastasis is inhibited in the presence of the test compound. In various embodiments, the decrease of the expression of the target is an indication that the test compound inhibits metastasis. In various embodiments, the decrease of the expression of the target, or its upstream signaling components, Src-kinase and Stat3 phosphorylation, are indications that the test compound inhibits metastasis. Alternatively, the RANKL pathway can be effectively blocked by the use of c-Met, VEGF-neuropilin-1, Src-kinase, Stat3 or Mcl-1 inhibitor and combinations thereof.

Various embodiments of the present invention provide for a method of switching osteolytic bone lesion and/or metastasis to osteoblastic bone lesion and/or metastasis in a subject in need thereof comprising: providing an agent capable of blocking RANK/RANKL signaling and/or HGF/cMet/VEGF/VEGFR2/neuropilin-1-mediated signaling; and administering the agent to the subject to switch osteolytic bone lesion and/or metastasis to osteoblastic bone lesion and/or metastasis.

In various embodiments, the agent is selected from the group consisting of denosumab,

RANK-Fc, OPG-Fc, siRNA, shRNA, XL-184, crizotinib, VEGFR2 kinase inhibitor III (CAS 204005-46-9) and combinations thereof. In certain embodiments, the siRNA or the shRNA inhibits RANKL expression.

In various embodiments, the present invention provides pharmaceutical compositions including a pharmaceutically acceptable excipient along with a therapeutically effective amount of an agent capable of inhibiting RANK and/or RANKL and/or an agent capable of inhibiting HGF-c-Met/VEGFR2/neuropilin-1-mediated signaling, including but not limited to downstream activation of neuropilin-1, Src-kinase, Stat3, Mcl-1, and NF-kB of the present invention. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection.

Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages of an effective an agent capable of inhibiting RANK and/or RANKL and/or an agent capable of inhibiting HGF-c-Met/VEGFR2/neuropilin-1-mediated signaling including but not limited to downstream activation of neuropilin-1, Src-kinase, Stat3, Mcl-1, and NF-kB of the present invention can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied malignant tumors, or the responses observed in the appropriate animal models, as previously described.

Embodiments of the present invention provide for a process, comprising: providing a composition comprising one or more detection probes to detect a protein selected from the group consisting of RANK, RANKL, and combinations thereof contacting the first composition to a tissue sample or a cell sample from a mammalian subject desiring a determination of cancer survival; quantifying the level RANK or RANKL to determine the likelihood of cancer survival in the mammalian subject. In various embodiments, the protein can be RANKL. In various embodiments, the cancer is a prostate, kidney, breast, bladder, lung, ovarian, pancreatic, thyroid, liver, gastric, colon or melanoma cancer.

In various embodiments, the one or more detection probe can comprise a label to produce a signal to detect the protein. In various embodiments, the label can be selected from the group consisting of a radiolabel, a chromophore, a fluorophore, a quantum dot and combinations thereof.

In various embodiments, the process can further comprise predicting a long survival time when a level of RANK or RANKL is lower than an established average, or predicting a short survival when a level of RANK or RANKL is higher than an established average. In various embodiments, the process can further comprise predicting a survival of over 100 months when a level of RANK or RANKL is lower than an established average, or predicting a of surviving less than 100 months when a level of RANK or RANKL is higher than an established average.

In various embodiments, the level of RANK or RANKL can be used to determine a course of therapy. In various embodiments, the course of therapy can comprise administering an agent capable of blocking RANK/RANKL signaling and/or HGF/cMet/VEGF/VEGFR2/neuropilin-1-mediated signaling. In various embodiments, the agent capable of blocking RANK/RANKL signaling and/or HGF/cMet/VEGF/VEGFR2/neuropilin-1-mediated signaling can be selected from the group consisting of denosumab, RANK-Fc, OPG-Fc, siRNA, shRNA, XL-184, crizotinib, VEGFR2 kinase inhibitor III (CAS 204005-46-9) and combinations thereof.

In various embodiments, the tissue sample or cell sample can be obtained from a tumor.

In various embodiments, the one or more detection probes can be an antibody capable of binding to RANK, RANKL, or combinations thereof.

Various embodiments of the present invention provide for a process, comprising providing a first composition comprising one or more isolated nucleic acid probes; contacting the first composition to an RNA sample from a mammalian subject desiring a determination of cancer survival, to produce one or more cDNA molecules; providing a second composition comprising one or more isolated nucleic acids probes comprising a sequence capable of hybridizing to one or more nucleic acids selected from the group of genes consisting of RANK and RANKL; contacting the second composition with the one or more cDNA molecules to amplify the one or more cDNA molecules; and quantifying the expression level of the one or more genes to determine the likelihood of cancer survival in the mammalian subject. In various embodiments, the one or more genes can be RANKL. In various embodiments, the cancer is a prostate, kidney, breast, bladder, lung, ovarian, pancreatic, thyroid, liver, gastric, colon or melanoma cancer.

In various embodiments, the process can further comprise predicting a long survival time when an expression level of the one or more genes is lower than an established average, or predicting a short survival when an expression level of the one or more genesis higher than an established average. In various embodiments, the process can further comprise predicting a survival of over 100 months when an expression level of the one or more genes is lower than an established average, or predicting a of surviving less than 100 months when an expression level of the one or more genes is higher than an established average.

In various embodiments, the expression level of the one or more genes can be used to determine a course of therapy. In various embodiments, the course of therapy can comprise administering an agent capable of blocking RANK/RANKL signaling and/or HGF/cMet/VEGF/VEGFR2/neuropilin-1-mediated signaling. In various embodiments, the agent capable of blocking RANK/RANKL signaling and/or HGF/cMet/VEGF/VEGFR2/neuropilin-1-mediated signaling can be selected from the group consisting of denosumab, RANK-Fc, OPG-Fc, siRNA, shRNA, XL-184, crizotinib, VEGFR2 kinase inhibitor III (CAS 204005-46-9) and combinations thereof.

In various embodiments, the RNA sample can be obtained from a tumor sample.

Various embodiments of the invention provide for a composition, comprising: one or more detection probes to detect a protein selected from the group consisting of RANK, RANKL, and combinations thereof; and a tissue sample or a cell sample from a mammalian subject desiring a determination of cancer survival. In various embodiments, the protein can be RANKL.

In various embodiments, the cancer is a prostate, kidney, breast, bladder, lung, ovarian, pancreatic, thyroid, liver, gastric, colon or melanoma cancer.

In various embodiments, the one or more detection probes can comprise a label to produce a signal to detect the protein. In various embodiments, the label can be selected from the group consisting of a radiolabel, a chromophore, a fluorophore, a quantum dot and combinations thereof.

In various embodiments, the one or more detection probes can be an antibody capable of specifically binding to RANK, RANKL, or combinations thereof.

Various embodiments of the invention provide for a composition, comprising: one or more isolated nucleic acids probes comprising sequences capable of hybridizing to the group of genes consisting of RANK and RANKL; and a tissue sample or a cell sample from a mammalian subject desiring a determination of cancer survival.

In various embodiments, the cancer is a prostate, kidney, breast, bladder, lung, ovarian, pancreatic, thyroid, liver, gastric, colon or melanoma cancer.

In various embodiments, the composition can further comprise: one or more isolated cDNA molecules transcribed from an RNA sample obtained from a mammalian subject desiring a determination of cancer survival.

Various embodiments of the invention provide for an array, comprising: a substrate having a composition comprising one or more isolated nucleic acids probes comprising sequences capable of hybridizing to the group of genes consisting of RANK and RANKL; or one or more isolated nucleic acids probes comprising sequences capable of hybridizing to the group of genes consisting of RANK and RANKL and one or more isolated cDNA molecules transcribed from an RNA sample obtained from a mammalian subject desiring a prognosis regarding a cancer.

In various embodiments, the cancer is a prostate, kidney, breast, bladder, lung, ovarian, pancreatic, thyroid, liver, gastric, colon or melanoma cancer.

Various embodiments of the invention provide for a process of prognosticating a tumor, comprising: providing one or more probes to detect the expression level of one or more genes or level one or more proteins from the group consisting of RANK, RANKL and combinations thereof; contacting the one or more probes to a test sample obtained from a mammalian subject desiring a prognosis regarding a tumor; determining one or more expression levels of the one or more genes or one or more proteins; prognosticating the tumor based on the one or more expression levels of the one or more genes or the level of the one or more proteins. In various embodiments, the one or more genes or one or more proteins can be RANKL.

In various embodiments, the cancer is a prostate, kidney, breast, bladder, lung, ovarian, pancreatic, thyroid, liver, gastric, colon or melanoma cancer.

In various embodiments, the one or more probes can be an antibody capable of specifically binding to RANK, RANKL, or combinations thereof, and determining one or more expression levels can be for the one or more proteins.

In various embodiments, the one or more detection probes can comprise a label to produce a signal to detect the protein. In various embodiments, the label can be selected from the group consisting of a radiolabel, a chromophore, a fluorophore, a quantum dot and combinations thereof.

In various embodiments, the process can further comprise predicting a long survival time when an expression level of the one or more genes or the level of the one or more protines is lower than an established average, or predicting a short survival when an expression level of the one or more genes or the level of the one or more proteins is higher than an established average. In various embodiments, the process can further comprise predicting a survival of over 100 months when an expression level of the one or more genes or level of the one or more proteins is lower than an established average, or predicting a of surviving less than 100 months when an expression level of the one or more genes or level of the one or more proteins is higher than an established average.

Various embodiment provide for a system, comprising: an isolated sample obtained from a mammalian subject desiring the prognosis of a cancer, the isolated sample comprising an abnormal level of RANK, RANKL, or combinations thereof; and a reagent to react with RANK, RANKL, or combinations thereof. In various embodiments, the isolated sample can comprise an abnormal level of RANKL and the reagent can be a reagent to react with RANKL.

In various embodiments, the cancer is a prostate, kidney, breast, bladder, lung, ovarian, pancreatic, thyroid, liver, gastric, colon or melanoma cancer.

In various embodiments, the reagent can further comprise a label to produce a signal to detect the abnormal level of RANK, RNAKL or combinations thereof. In various embodiments, the label can be selected from the group consisting of a radiolabel, a chromophore, a fluorophore, or a quantum dot.

The present invention is also directed to a kit to treat cancer, a kit to identify a compound that inhibits metastasis, and a kit to prognosticate cancer. The kit is useful for practicing, for example, the inventive method of treating cancer or identifying a compound that inhibits metastasis. The kit is an assemblage of materials or components, including at least one of the inventive compositions. Thus, in some embodiments the kit contains a composition including an agent capable of inhibiting RANK and/or RANKL and/or an agent capable of inhibiting HGF-c-Met/VEGFR2/neuropilin-1-mediated signaling, including but not limited to downstream activation of neuropilin-1, Src-kinase, Stat3, Mcl-1, and NF-kB of the present invention, as described above. In some embodiments, the kit contains one or more compositions as discussed above to prognosticate cancer.

The exact nature of the components configured in the inventive kit depends on its intended purpose. For example, some embodiments are configured for the purpose of treating cancer. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat cancer, identify agents that inhibit metastasis. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Cell Culture

ARCaP_{E} and ARCaP_{M} cells were cultured and grown in T-medium supplemented with 10% FBS and 1% Penicillin. The cells were grown to 90% confluence before assay for cell extracts.

### Example 2

### Transfection

4×10⁵ ARCaP and PC3 cells were seeded into a 6-well plate and cultured for 48 hrs until they grow to 90% confluence. The RANKL siRNA (50 pmole) and siRNA control (50 pmole) and lipofetamine (2.5 ul) were mixed with 250 ul of OPTI-medium without any FBS, respectively and incubated at RT for 5 minutes. The DNA and lipofetamine were then mixed together and incubated for 20 minutes before adding to the cells in each well. The medium was changed to normal 5% T-medium after 6 h incubation. The cells were incubated at 37° C. for two days before harvesting.

### Example 3

### Protein Extraction

The cells in a 10 cm dish were scraped off in the medium and transferred to 1.5 ml Eppendorf tubes. After centrifugation at 1,800 rpm for 5 minutes, the supernatant was removed and 100 ul of Triple Detergent Lysis Buffer (with 1.5 ul PMSF and 6 ul of 25× protease inhibitor) was added to the cell pellets. The lysate was mixed and incubated on ice for 30 minutes with constant vortexing followed by centrifugation at 14,000 rpm, 10 minutes, at 4° C. The supernatant was then transferred to 0.5 ml Eppendorf tube, and the protein concentration was measured.

### Example 4

### Western Blotting

Total of 30 ug of proteins per well from both ARCaP_{E} and ARCaP_{M} cells were run on a Tris-Bis 4-12% gradient polyacrylamide gel (Invitrogen) one and half hours at 150 Volts. The proteins on the gel were then transferred to PVDF membranes using Hoefer semi-dry system. The membranes were incubated with 5% skim milk in PBST (Blocking solution) for 20 min to block non-specific antibody labeling. The RANKL primary antibody was diluted with blocking solution (1:200 and 1:500) and incubated with the membrane for one hour at RT on a rocker or overnight at 4° C. The membrane was washed with PBST (PBS+0.01% TWEEN) three times for a minimum of 5 min each. Secondary rabbit anti-goat antibodies were incubated with the membrane for one hour at RT on a rocker. The membrane was again washed with PBST three times for 5 min each. Chemiluminescent substrate (Western Lightning) was evenly added across the top surface of the membrane, and the membrane was exposed to X-ray films for 30 seconds to 2 minutes depending on the strength of the signals and then developed according to the manufacturer's instructions.

### Example 5

### Reverse Transcription

2 ul of RNA from each cell, 3 μl Random Hexamer (RH), 1 ul of 10 mM dNTP mix and 4 ill DEPC water were mixed in a PCR tube and labeled as Mix A. The reaction mixture was heated at 65° C. for 5 min to separate secondary structures from the RNA, then put on ice for 1 min to allow the Random Hexamer (Invitrogen) primers to anneal to single-stranded RNA. In a separate tube, 2 μl of 10× buffer, 4 μl 25 mM MgCl₂, 2 μl of 0.1M DTT, 1 μl RNaseH, and 1 μl of reverse transcriptase (200 U/μl) from the Superscript First Strand kit (Invitrogen) were added, mixed, and labeled as Mix B. Mix B was transferred into Mix A and left at RT for 10 min. The cDNA reaction mixture was heated at 42° C. for 60 min and then at 7° C. for 15 min to inactivate the reverse transcriptase.

### Example 6

### CDNA Amplification

For each reaction, a 50 μl PCR reaction mix containing 5 μl of 10×PCR buffer, 1.5 ul of 50 mM MgCl₂, 1 ul of 10 mM dNTP, 1 ul of each forward and reverse primers (RANKL F: CAGCACATCAGAGCAGAGAAAG (SEQ ID NO:1) and RANKL R: TGTTGGCATACAGGTAATAAAAGC (SEQ ID NO:2); GAPDH as the positive control), 3 ul cDNA, 37 ul double distilled water, and 0.5 μl of Taq polymerase (Invitrogen) was made. The reaction was first activated at 94° C. for 2 min and run for 36 cycles of 94° C. (30 sec-melting), 60° C. (30 sec-annealing), and 72° C. (60 sec-extension), and lastly incubated at 72° C. for 5 min to amplify the cDNA. A 1% gel electrophoresis was run with 5 μl of 100 by DNA ladder and 10 μl of amplified cDNA of each sample to check the size of the cDNA and also whether it was successfully synthesized under UV light.

### Example 7

### RANKL Treatment

The cells are seeded in 6-well plate and 24 h later, the cells are serum-starved for one day in RPMI-1640 medium, and 200 ng/ml of RANKL and 400 ng/ml of OPG were added to the cells and incubated for three to four days before harvest.

### Example 8

### Endogenous Expression of RANKL, RANK, and OPG in Prostate Cancer Cells

The inventors first identify the endogenous expression of RANKL/RANK/OPG axis in different prostate cancer cells as well as the positive control osteoblast cell, SaOS-2. Most prostate cancer cells express RANKL and RANK, not all prostate cancer cells express OPG or express at very different levels.

### Example 9

### ARCaP_{E} Treated with RANKL

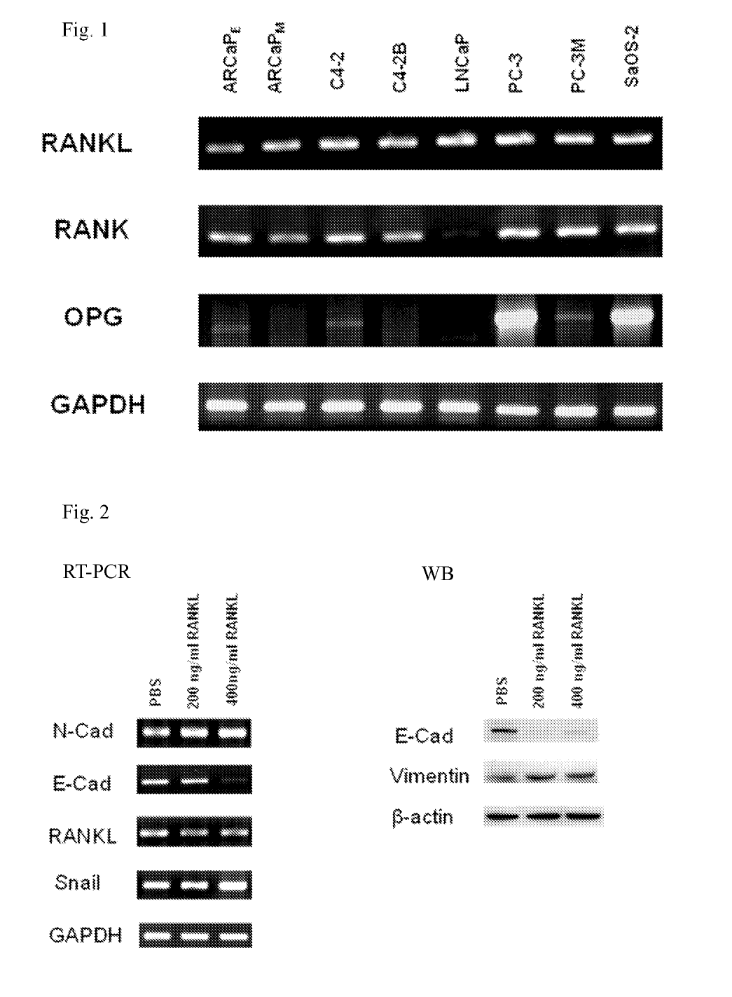

To demonstrate that RANKL can drive ARCaP_{E} cells undergoing EMT, the inventors treated the cells with recombinant RANKL (200 and 400 ug/ml) for four days, and both mRNA and protein levels of different EMT markers were examined by RT-PCR and Western blot. The N-cadherin expression is slightly increased by RANKL and decrease of E-cadherin is not so dramatic in RNA level compared to the protein level. RANKL seems to have no effect on the expression of Snail but increases the vimentin expression (FIG. 2).

### Example 10

### ARCaP_{E} Treated with RANKL Plus OPG

OPG is the decoy receptor of RANKL, and it is known to inhibit RANKL induced downstream signaling. Therefore, the ARCaP_{E} cells were treated with both RANKL and RANKL plus OPG to examine the expression of the EMT markers. From RT-PCR, N-cad and Vimentin are slightly decreased by OPG compared to the cells treated with 300 ng/ml RANKL, and the OPG could restore the expression of E-cad. The decrease of E-cad again was much more obvious in WB and OPG could restore the E-cad expression. Vimentin was only slightly increased by RANKL but significantly reduced by OPG.

### Example 11

### Overexpression of RANKL In ARCaP Cells

An alternative approach is applied to examine the role of RANKL in inducing EMT in prostate cancer cells. RANKL cDNA is subcloned into p3×Flag vector (Sigma) and the Flag-tag is at the N-terminus and the plasmid is then transiently overexpressed in ARCaP_{E} cells. EMT markers are again examined with RT-PCR and Western blot. RANKL expression vector increased N-cadherin expression compared to the neo control and E-cadherin expression is also decreased by RANKL expression in ARCaP_{E} cells. The overexpression of RANKL is confirmed by RT-PCR. In this case, RANKL expression was able to increase Snail expression at RNA level and Vimentin at protein level (FIG. 4).

### Example 12

### Transient RANKL Knockdown in ARCaP_{M} Cells

RANKL expression can drive EMT in ARCaP_{E} cells to become more mesenchymal like. Another approach to prove that RANKL can regulate EMT in ARCaP cells is to knockdown RANKL in a more mesenchymal-like ARCaP_{M} cells to determine if the expression of mesenchymal markers can be dampened.

With 20 pmole RANKL siRNA, only slight decrease in N-cadherin expression but more E-cadherin expression, meaning that the cells were now becoming more epithelial-like. However, the knockdown of RANKL is not that impressive, only to about 50% knockdown (FIG. 5).

### Example 13

### RANKL Promoter Construct

Because the inventors are interested in how RANKL is transcriptionally regulated, the inventors have constructed a 2.5 kb 5′ upstream region of RANKL promoter. The inventors used BAC clones (RP11-86N24) to amplify the promoter region of RANKL from human chromosome 13 contig. The whole promoter region (2.5 kb) was then cloned into pGL3 basic luciferase reporter vector. The inventors placed RANKL promoter reporter into ARCaP_{E,M}, C4-2, and a positive cell line, SaOS-2 to examine the basic promoter activity. Then, the inventors stimulated the RANKL promoter by various growth factors, such as vitamin D₃, PTH, and TGFβ.

### Example 14

PCa cell lines and clinical specimens express bone-specific proteins that collectively confer osteomimetic properties. Osteomimicry plays important roles supporting PCa growth and survival in bone [14-17]. Responding to growth factors, cytokines and chemokines in the tumor microenvironment, PCa cells will undergo EMT to acquire the ability to metastasize, as the inventors have demonstrated in LN and ARCaP PCa progression models [18-24]. The inventors evaluate specifically the biology of RANKL- and HGF-signaling axes and the interactions of PCa cells with OCs and OBs, to provide a rationale for targeting these converging signaling networks and developing novel predictive biomarkers for PCa progression. This approach is taken because the inventors discovered an expansion of a RANKL/RANK/OPG triad relationship to c-Met signaling in which RANKL not only has a forward feedback loop with regard to RANKL expression but also an extension toward c-Met expression. This observation has important clinical implication establishing the concept of targeting RANKL, c-Met and their closely related converging cell signaling network involving but not limited to downstream activation of neuropilin-1, Src-kinase, Stat3, Mcl-1, and NF-kB.

### Example 15

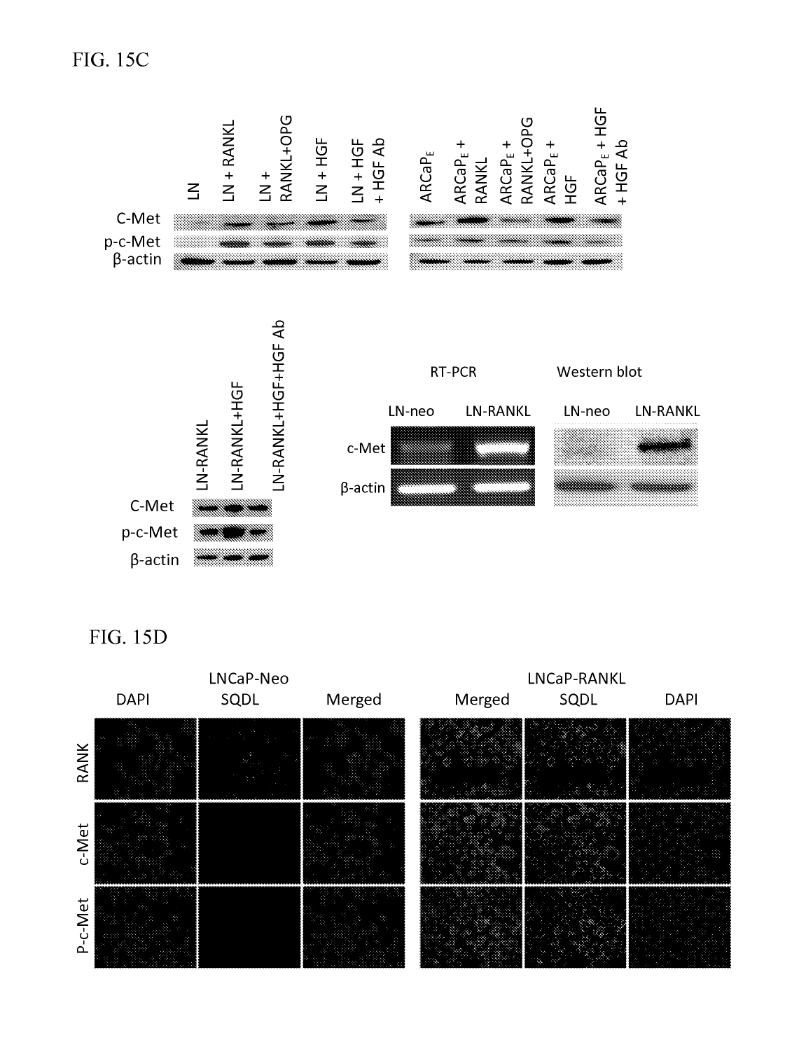

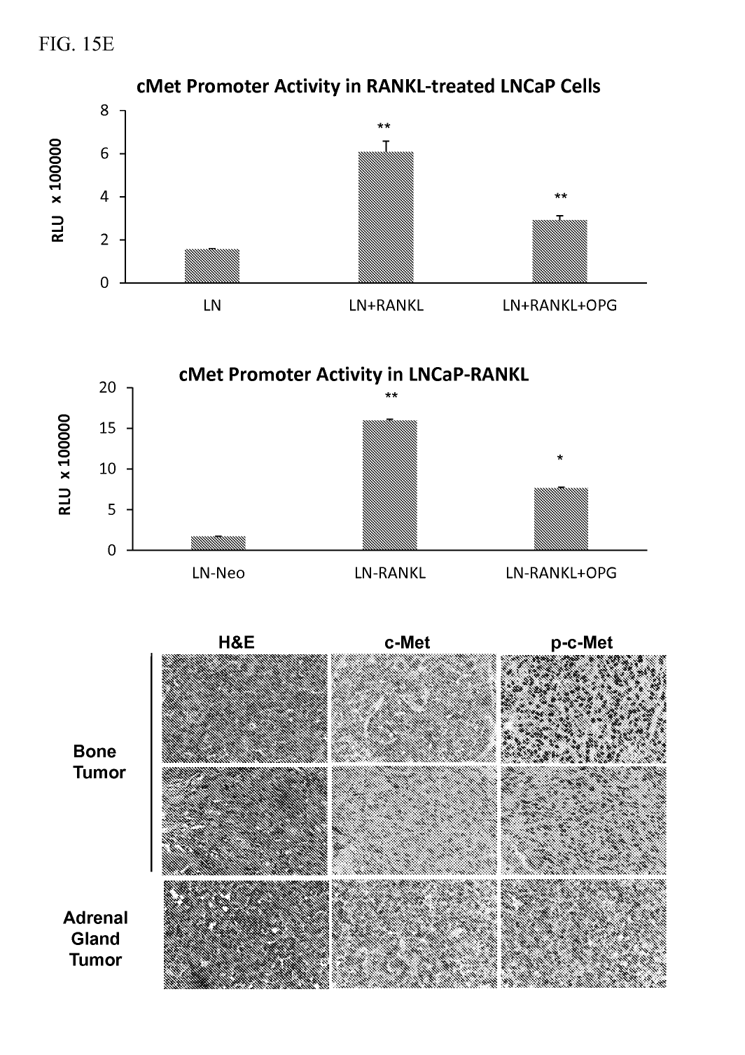

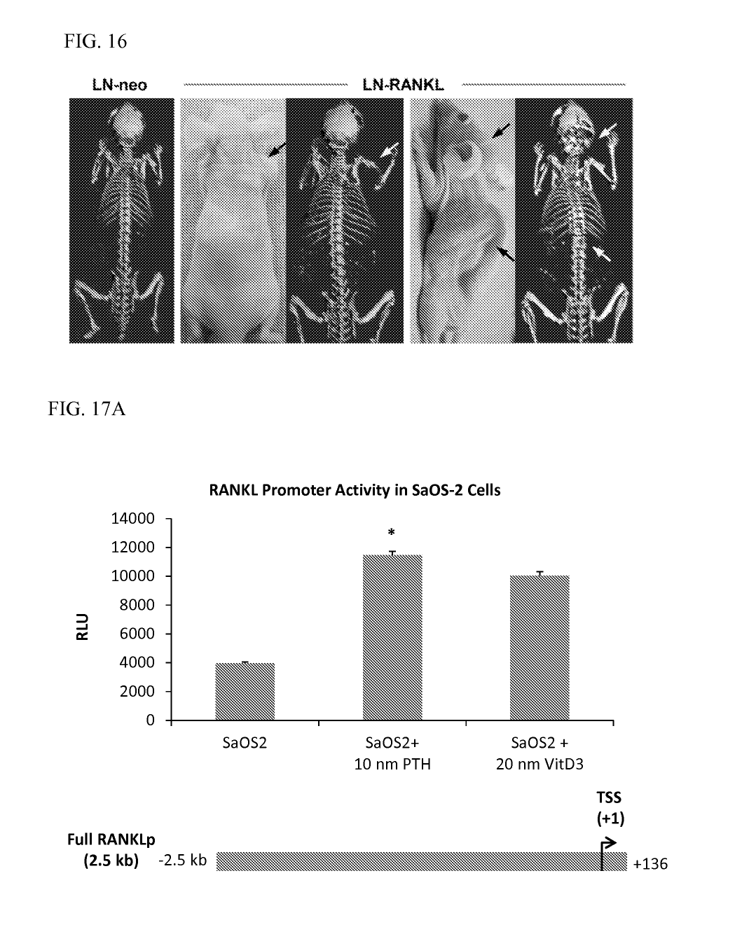

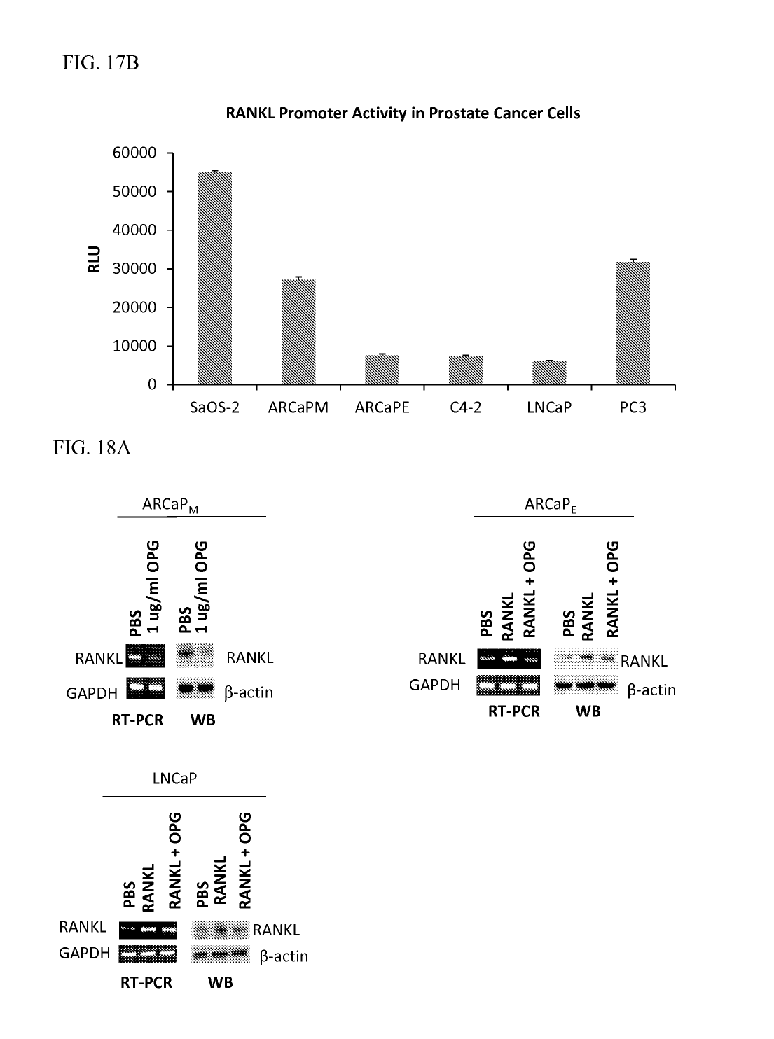

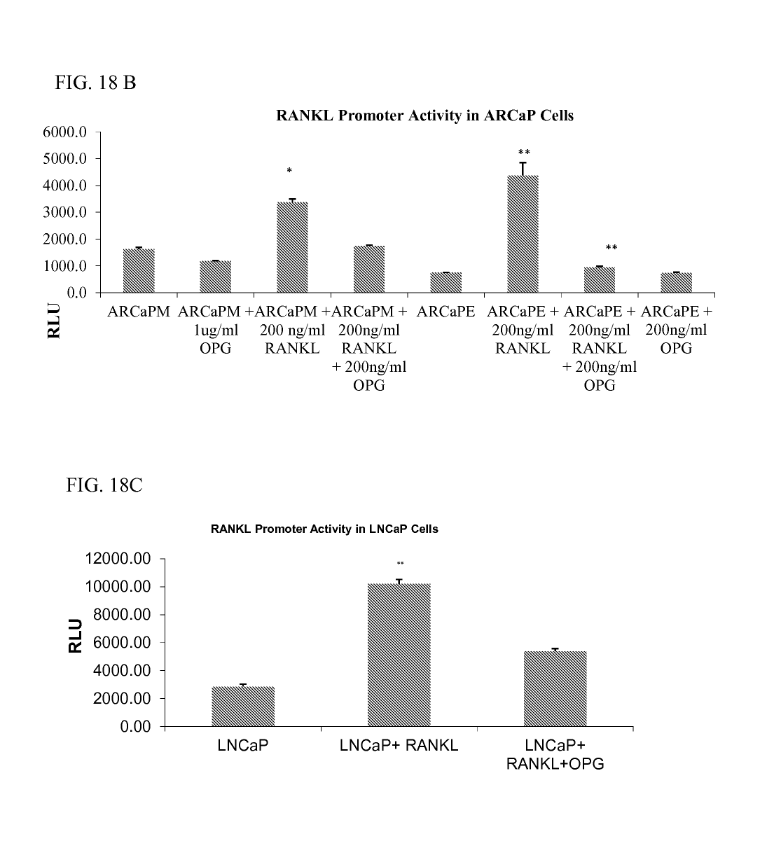

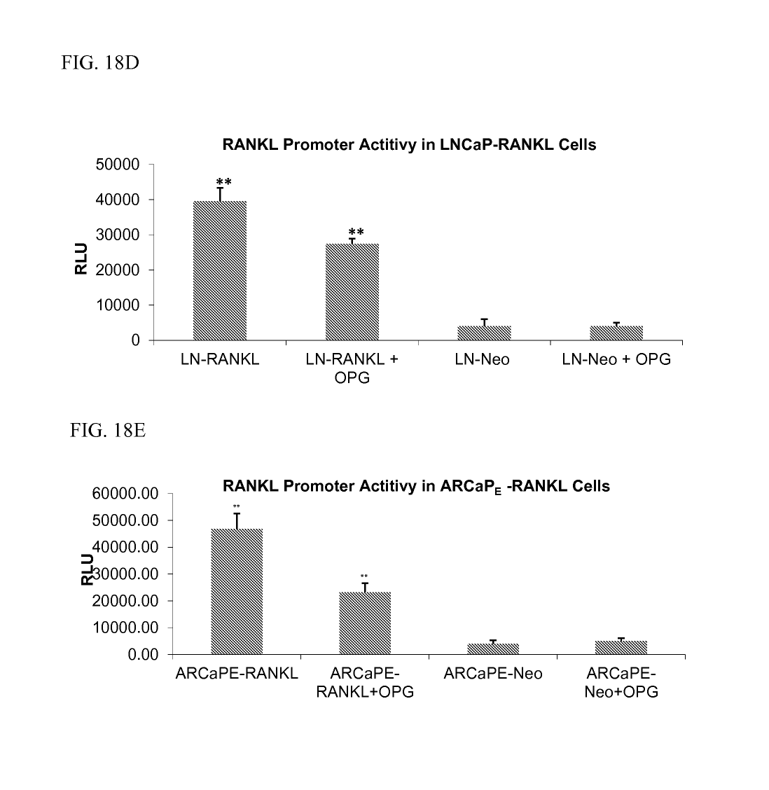

LN cells express constitutively a low level of RANKL and do not form xenograft tumors when inoculated in the absence of Matrigel or organ-specific stromal cells [22, 24-27]. Likewise, LN tagged with a red fluorescence protein (LN^{RFP}) did not form tumors during a 14-month period (data not shown). In ARCaP_{M} cells, the RANKL expression induces EMT and metastases, mediated by Snail and ROS [3, 18, 19]. RANKL was stably expressed in PCa cells to characterize the morphology, gene expression, and behavioral changes. LN stably expressing RANKL (LN-RANKL) underwent EMT, upregulated N-cad and vimentin, and down-regulated E-cad (FIG. 15). LN-RANKL cells injected intracardially (IC) in mice metastasized to bone, lymph node, lung and adrenal gland at 100, 90, 40 and 85% frequency, respectively (Table 1). MicroCT and X-ray radiography showed that the tumors formed in mouse bone were primarily osteolytic, mixed with osteoblastic lesions (FIG. 16). Clinical relevance of the RANKL- and HGF-axes were determined with a quantum-dot (QD)-based multiplex IHC protocol to collect evidence of EMT in primary and bone metastatic human PCa specimens. It was found that EpCAM-positive (epithelial) and negative (transitioned to mesenchyme) PCa cells co-existed but expressed both RANKL and N-cad, both are the markers of mesenchymal transition. Similarly, increased RANKL, N-cad, and phosphorylated c-Met were identified in the novel LTL313 CRPC xenograft model and clinical CRPC specimens. This QD-based multiplex IHC procedure will be shared to analyze multiple gene expression simultaneously at the single cell level.

TABLE 1
RANKL overexpression renders metastatic potential to LNCaP cells

| | Bone | Lymph node | Adrenal gland | Lung |
|---|---|---|---|---|
| LN-neo | 0/15 | 0/15 | 0/15 | 0/15 |
| LN- | 20/20 (100%) | 18/20 (90%) | 17/20 (85%) | 8/20 (40%) |
| RANKL | | | | |

### Example 16

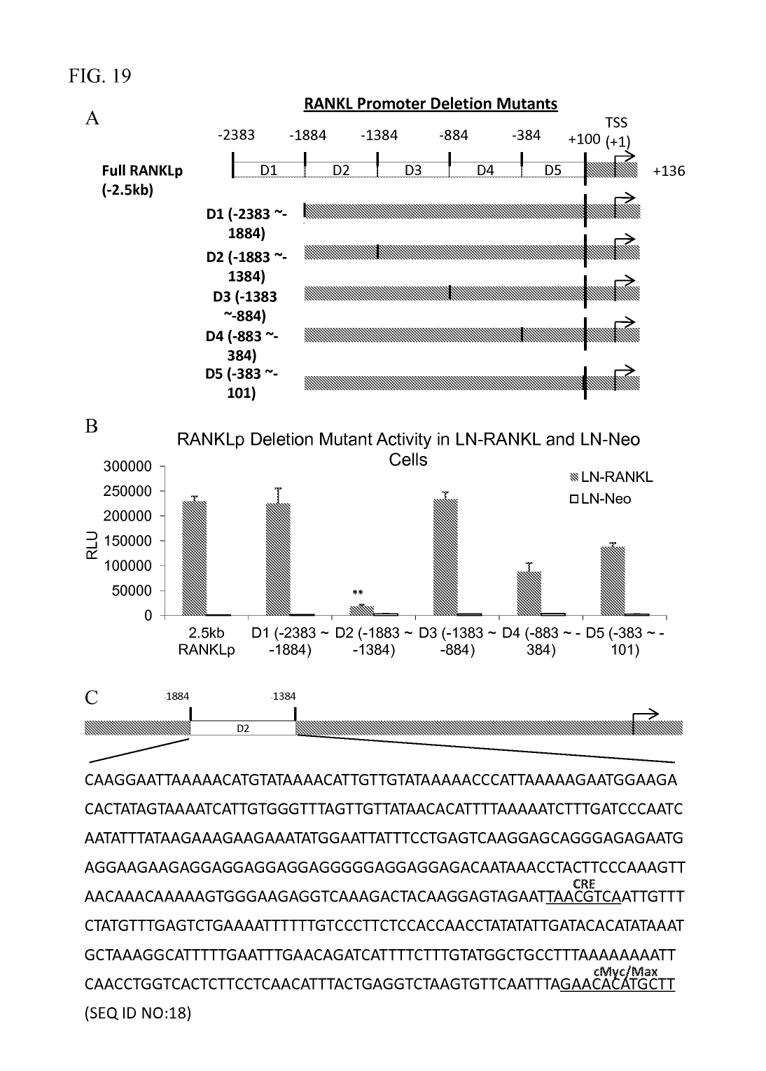

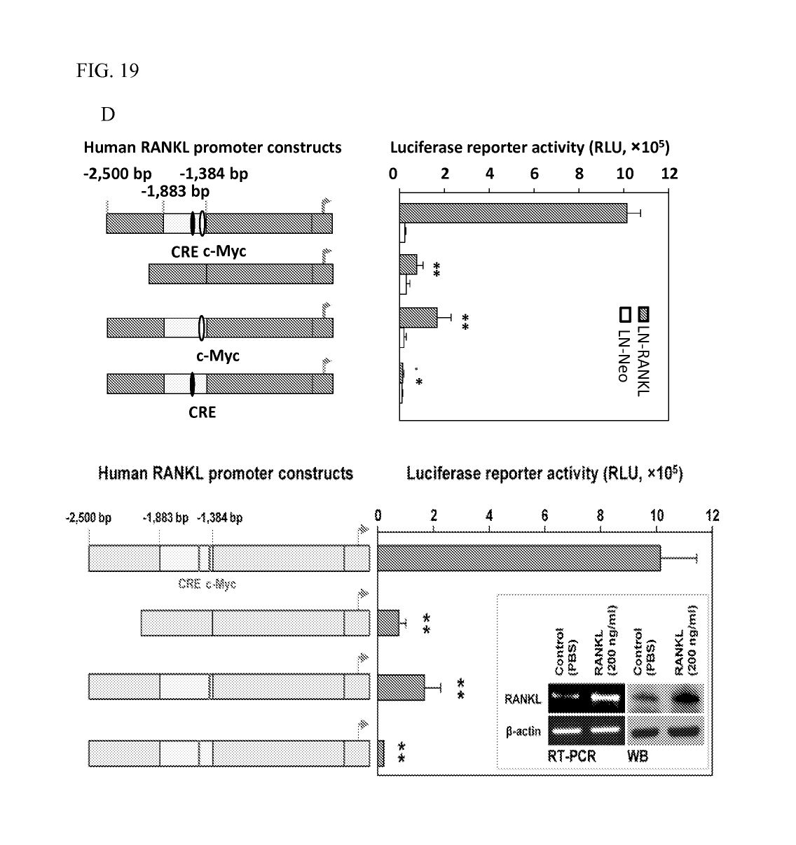

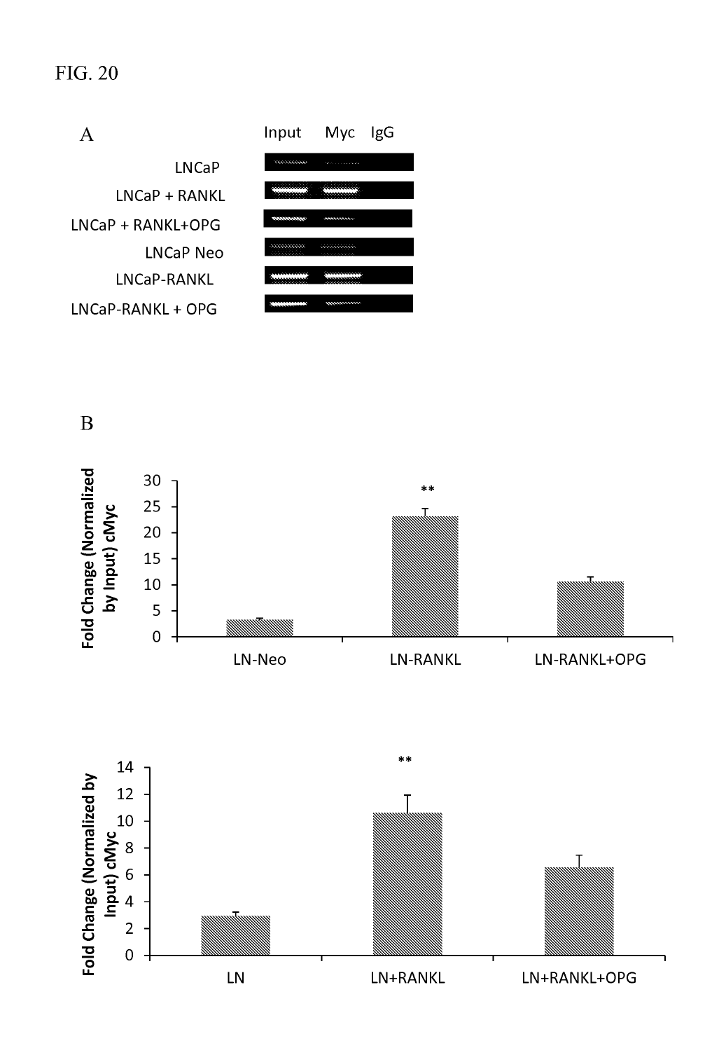

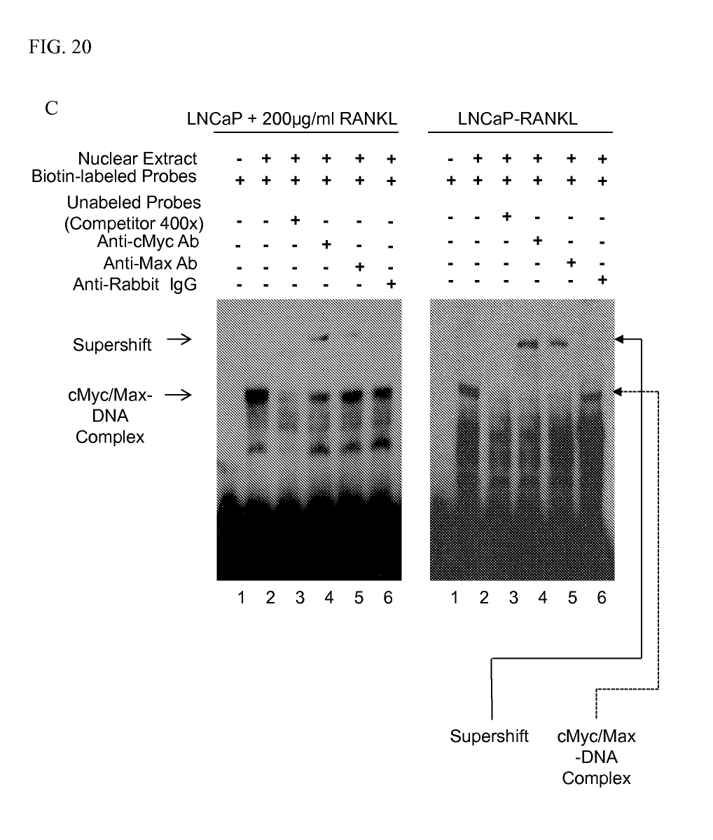

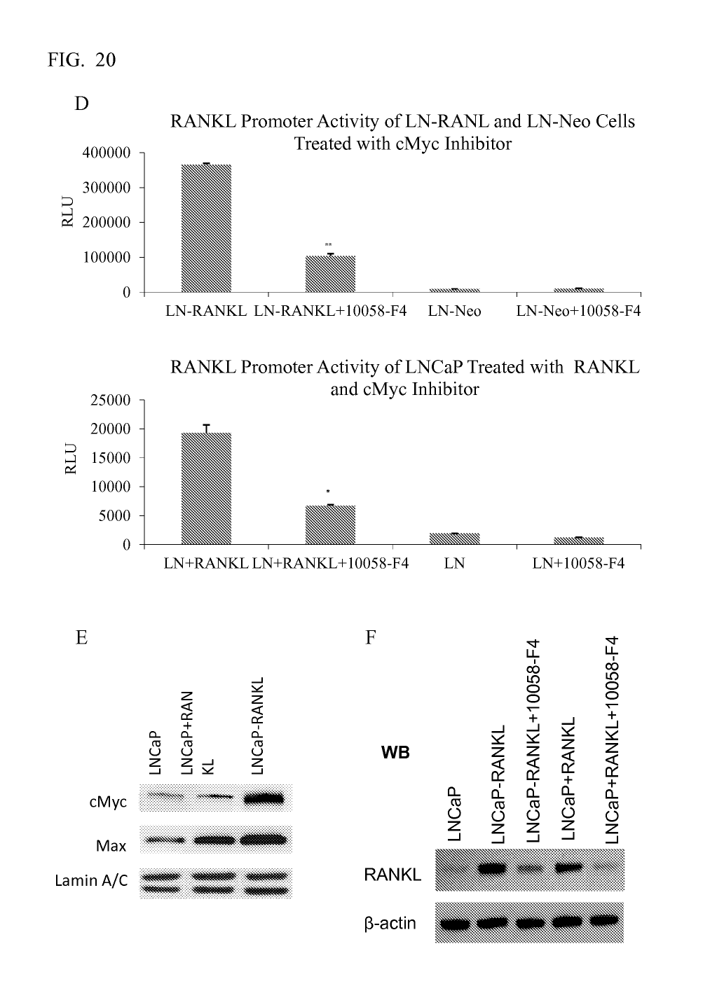

It was found that treating LN cells with RANKL protein induced endogenous RANKL expression (FIG. 19), and RANKL-overexpression in PCa cells led to increased endogenous RANKL (FIG. 15). To study the autocrine regulation, the inventors cloned a 2.5 Kb human RANKL promoter and engineered deletion constructs driving a luciferase reporter. This series of study identified CREB and c-Myc binding cis-elements as mediators in the autocrine regulation of RANKL expression (FIG. 19). RANKL expressed by PCa cells was biologically functional, since it potently induced TRAP⁺ OC formation by the Raw264.7 mouse pre-osteoclast cell line, and since the induction could be blocked by OPG, a decoy receptor for RANKL protein (FIG. 16).

### Example 17

It was found that RANKL markedly induced the expression of c-Met in PCa cells (FIG. 15), revealing a converging signaling pathway between the RANKL- and HGF-axes with a possible VEGF participation downstream. It was previously reported that VEGF binds to a co-receptor, neuropilin-1, which complexes with c-Met to facilitate c-Met phosphorylation and to activate the anti-apoptotic gene of myeloid cell leukemia-1 (Mcl-1) [28]. Mcl-1 induction involves rapid activation of Src-kinase and Stat3. The inventors also established that PDGF-BB, an osteogenesis factor, is a target of the RANKL axis and activates Mcl-1 through HIF-1α, which forms a complex with β-catenin and p68 and activates the Mcl-1 promoter (data not shown). Collectively, these data suggest that Mcl-1 can be used as a read-out for activation of the converging signaling pathway between the RANKL/RANK- and HGF/c-Met-axes. Both of the signaling mechanisms involved downstream activation of p38, MAPK, PI3K-Akt and NF-kB. Whether c-Met activation can reciprocally activate RANKL-RANK signaling is under investigation.

### Example 18

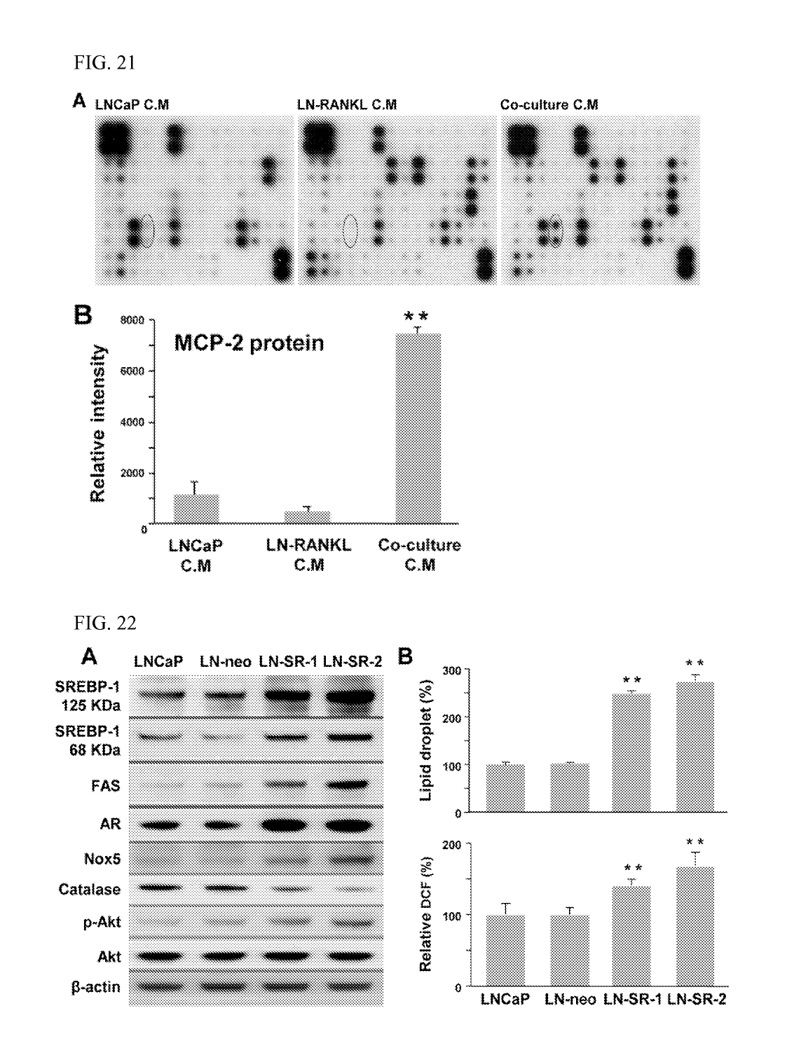

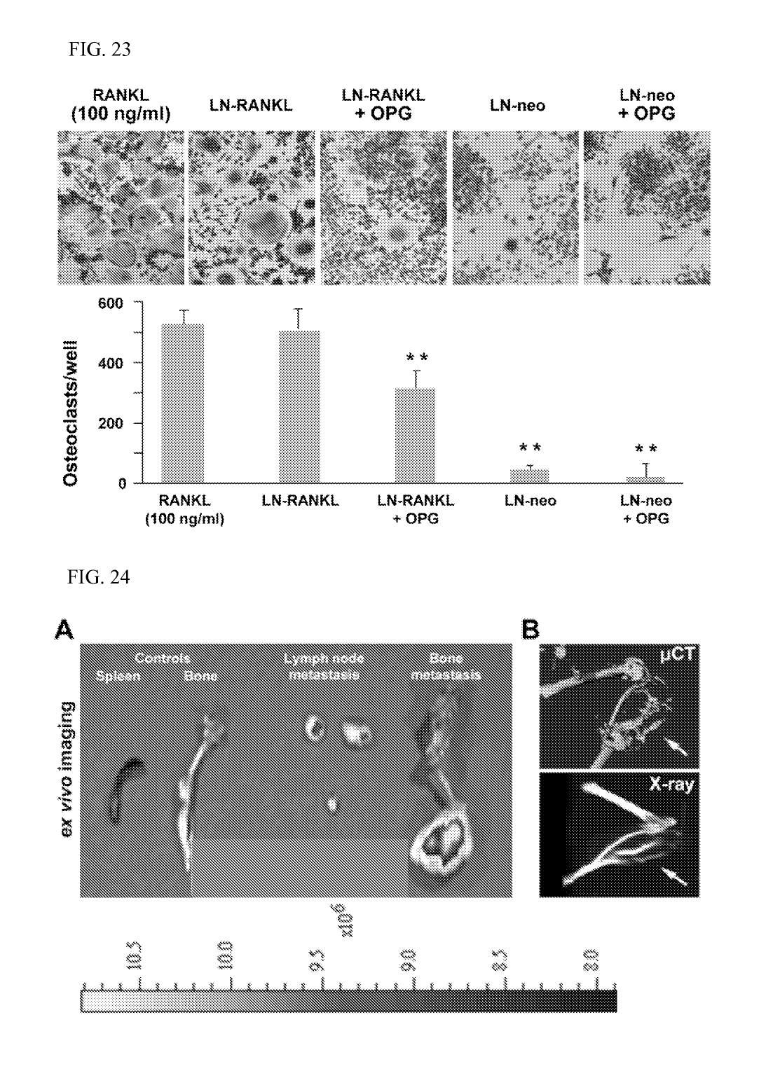

Xenograft inoculation of LN-RANKL cells caused predominantly osteolytic bone metastasis, with only occasional osteoblastic reactions being detected. Intriguingly, abundant osteoblastic responses were produced when LN-RANKL cells were co-inoculated with the parental LN cells at a 1:9 ratio (a chimeric LN model). To understand the switch, the inventors conducted cell co-culture to harvest conditioned media (C.M) from LN-RANKL, LN and the co-cultured LN-RANKL:LN cells. Secreted soluble growth factors in the conditioned media were identified with the RayBio Human Cytokine Antibody Array system (FIG. 21). As an example, such an assay revealed a specific induction of MCP-2 protein, a chemotaxis factor, in the mixed cell co-culture. The inventors analyzed the expression of other cytokines and chemokines using Cytokine Antibody Arrays and a parallel qRT-PCR array (SABioscience). These studies indicated 5 categories of soluble growth factors, which function in mediating chemotaxis, bone resorption, bone tissue formation, proteolytic cleavage and angiogenesis, were upregulated in the LN-RANKL cells (Table 2). Taken together, these studies demonstrate that an increased RANKL level in PCa cells can alter production and secretion of chemokines, cytokines and growth factors. The alteration could affect the balance of osteolytic/osteoblastic activities when PCa cells colonize bone. The secreted factors will be further refined, validated and developed as predictive biomarkers for PCa progression. Importantly, these factors produced by the mixed population of PCa cells could also determine the status of dormancy of PCa cells since LN failed to form bone metastasis in mouse skeleton alone but requiring the participation of LN-RANKL cells.

TABLE 2
RANKL-induced expressional changes of soluble factors.

| Chemotaxis | Bone resorption | | Bone formation | | Proteolytic activation | | Angiogenesis | |
|---|---|---|---|---|---|---|---|---|
| MCP-2 | HGF | BMP-3 | OPG | FGF-1 | TIMP-1 | MMP8 | VEGF | RANKL |
| GRO | IL-6 | TGFβ1 | TIMP-1 | FGF-2 | uPAR | MMP9 | CD40 | TFNα |
| GCP-2 | IL-8 | RANKL | PDGF-BB | BMP-4 | | MMP10 | CD40 | FGF-1/2 |
| | | | | | | | LIGAND | |
| GM-CSF | | | Runx2 | BMP-5 | | Cathepsin K | MMP9 | TGFβ1 |

### Example 19

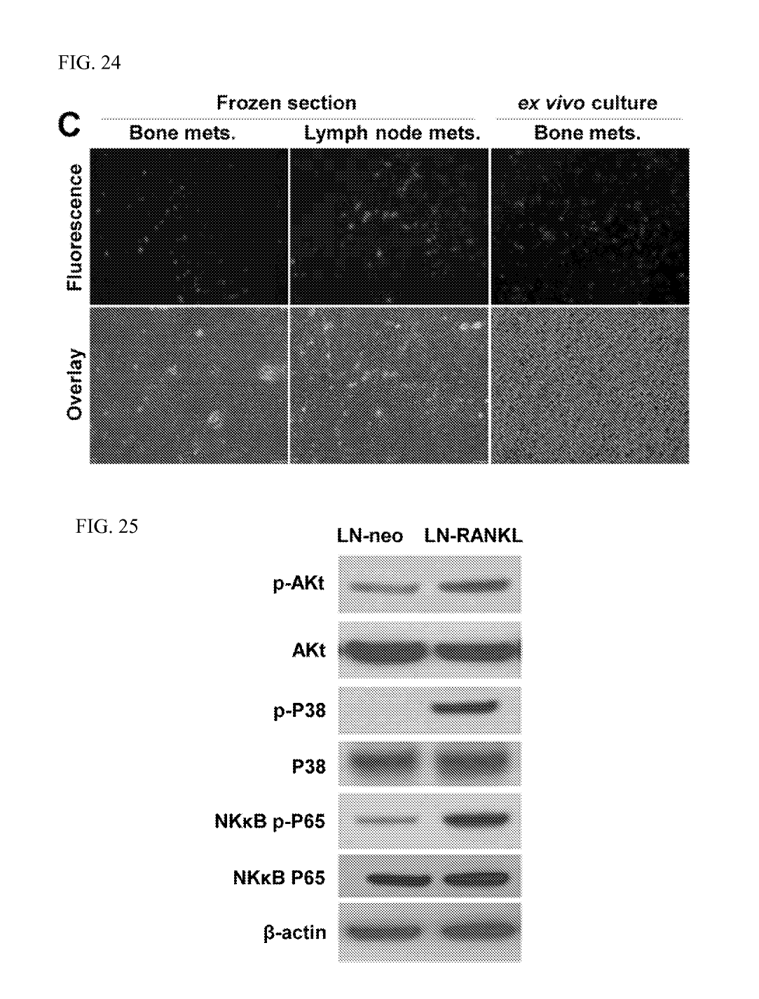

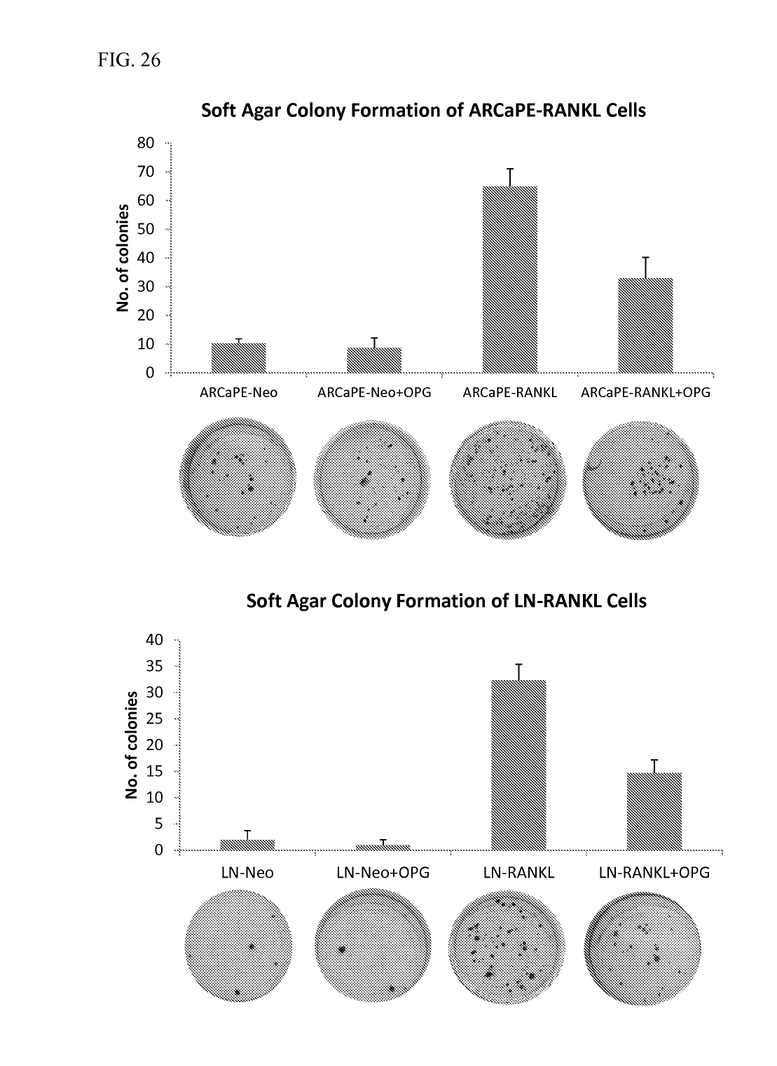

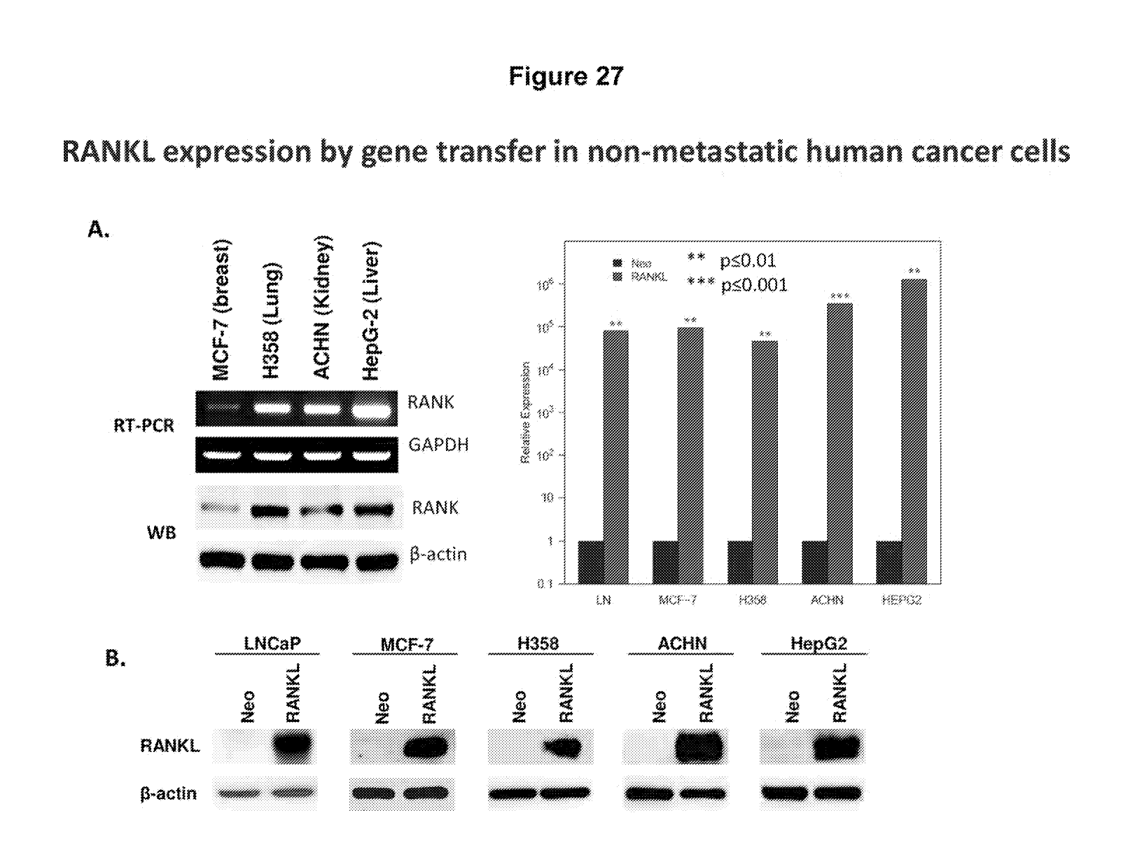

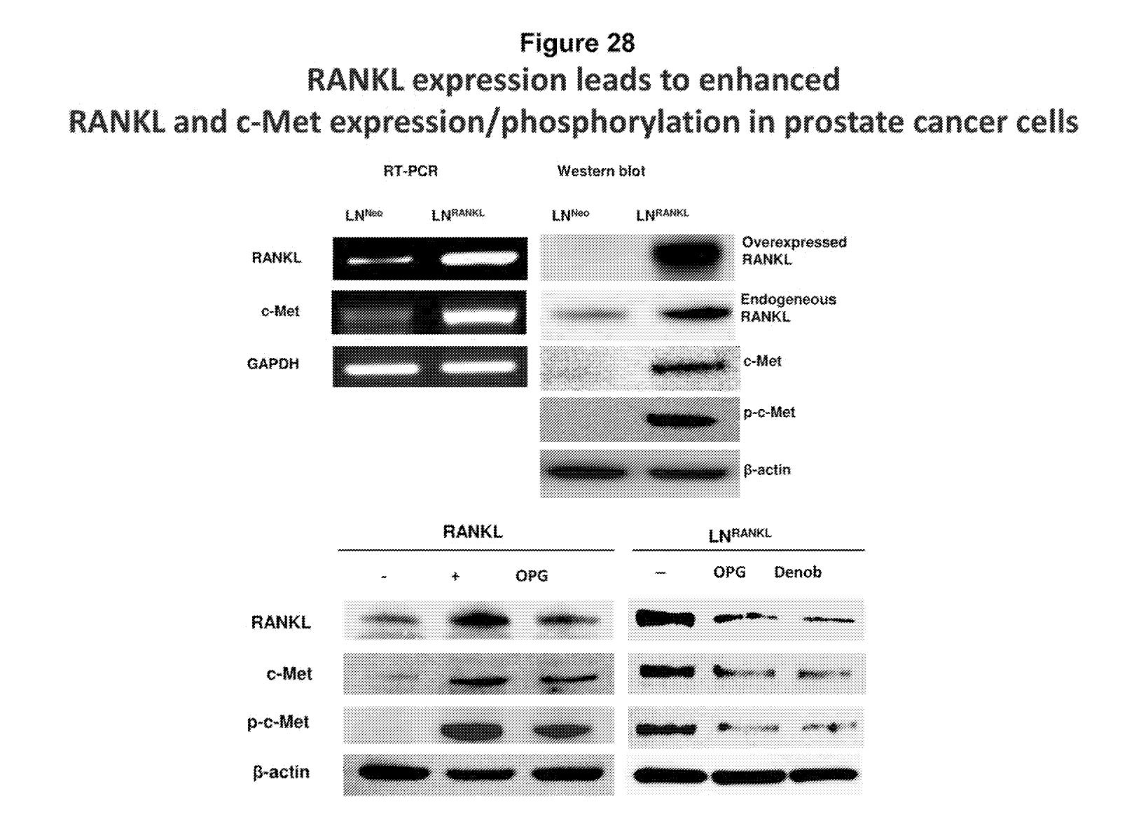

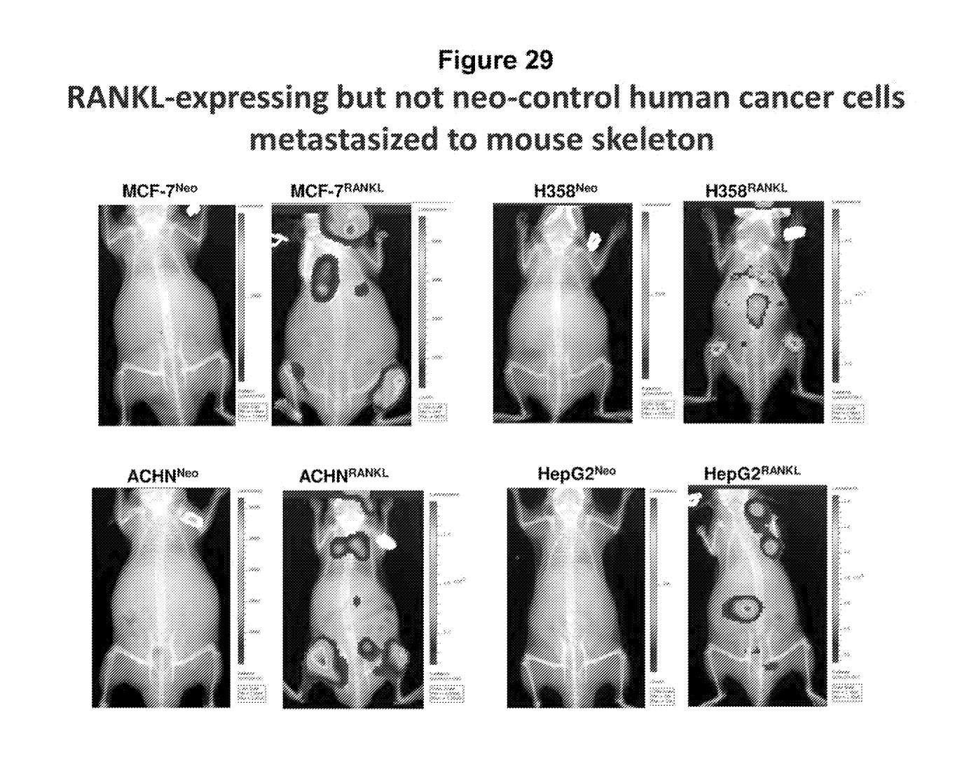

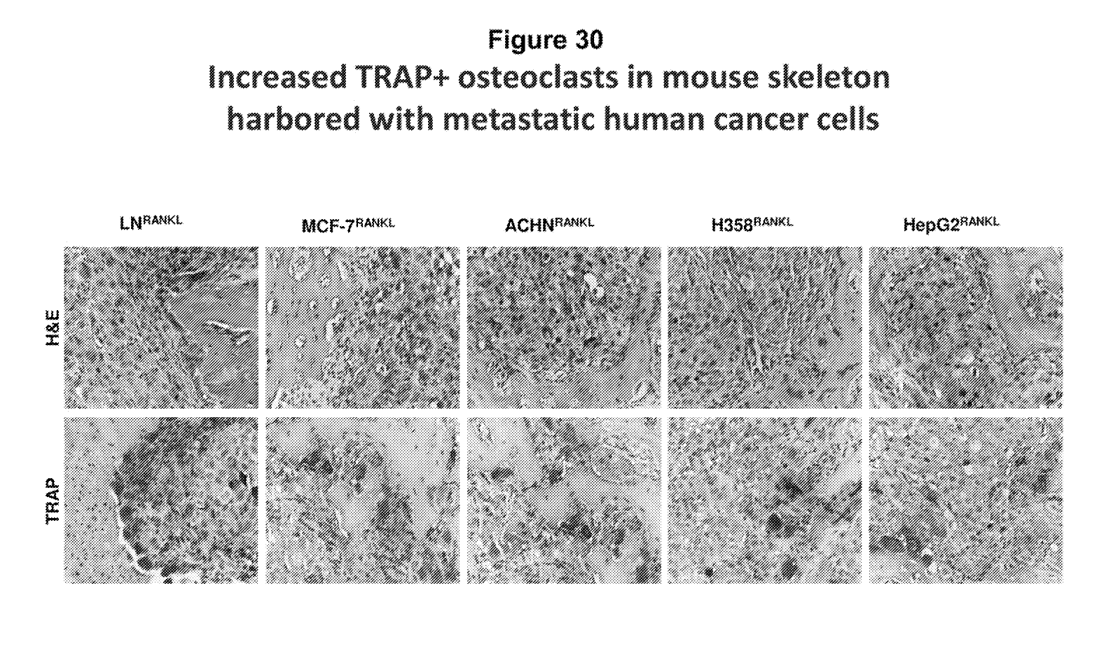

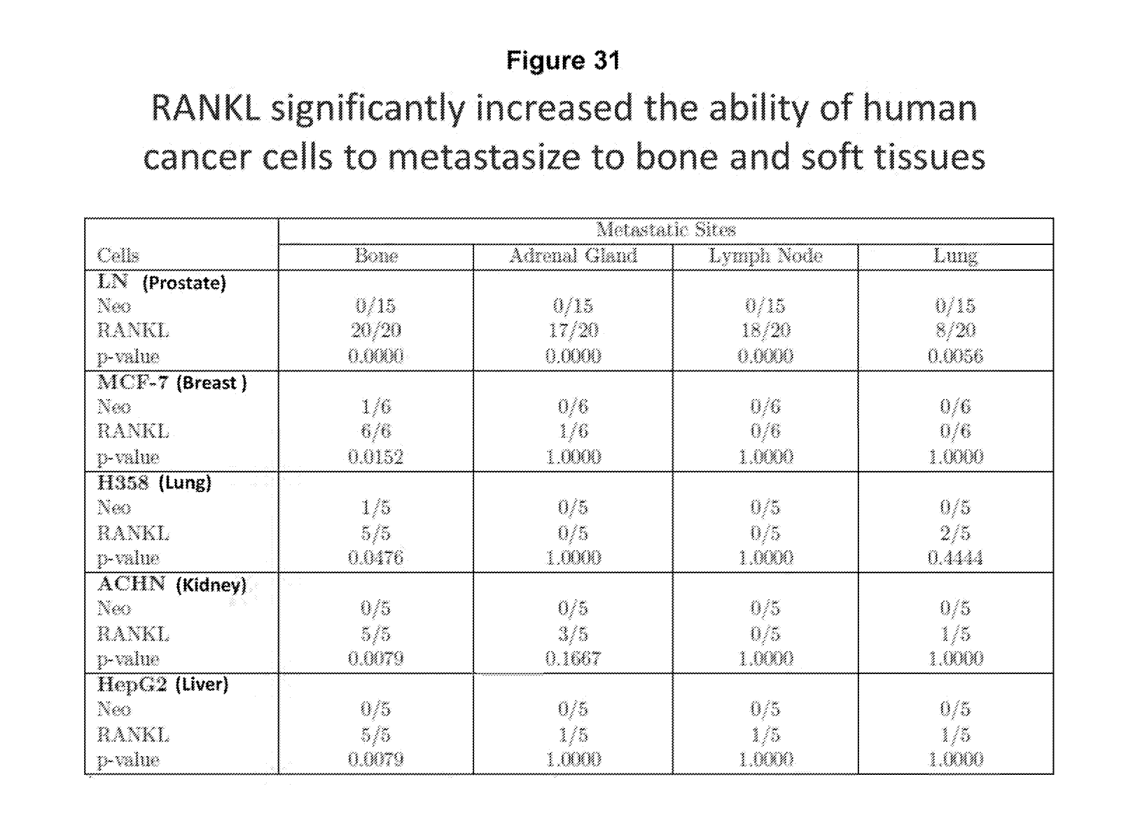

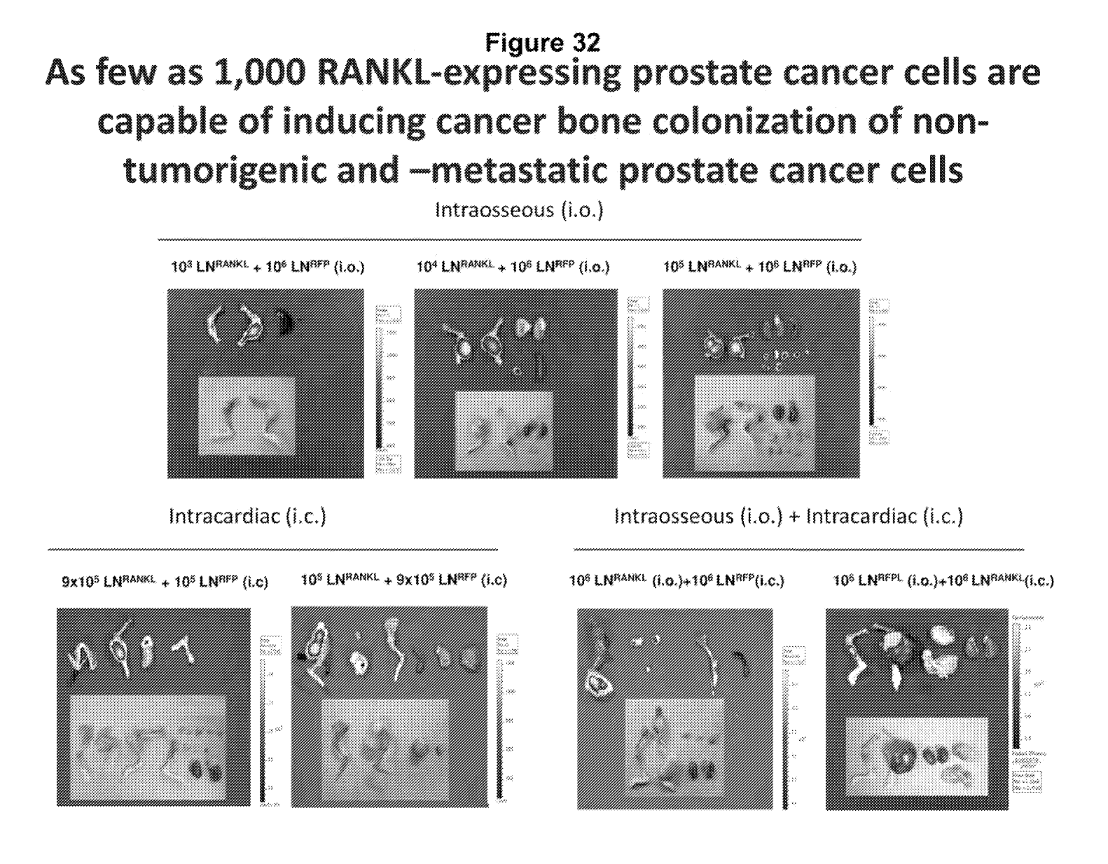

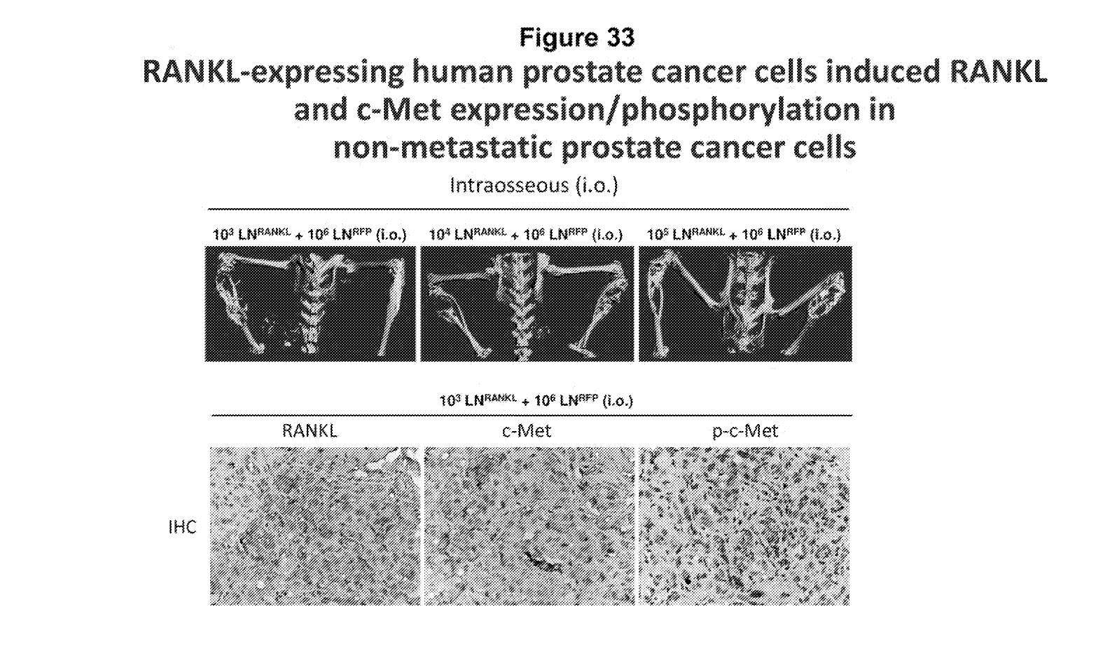

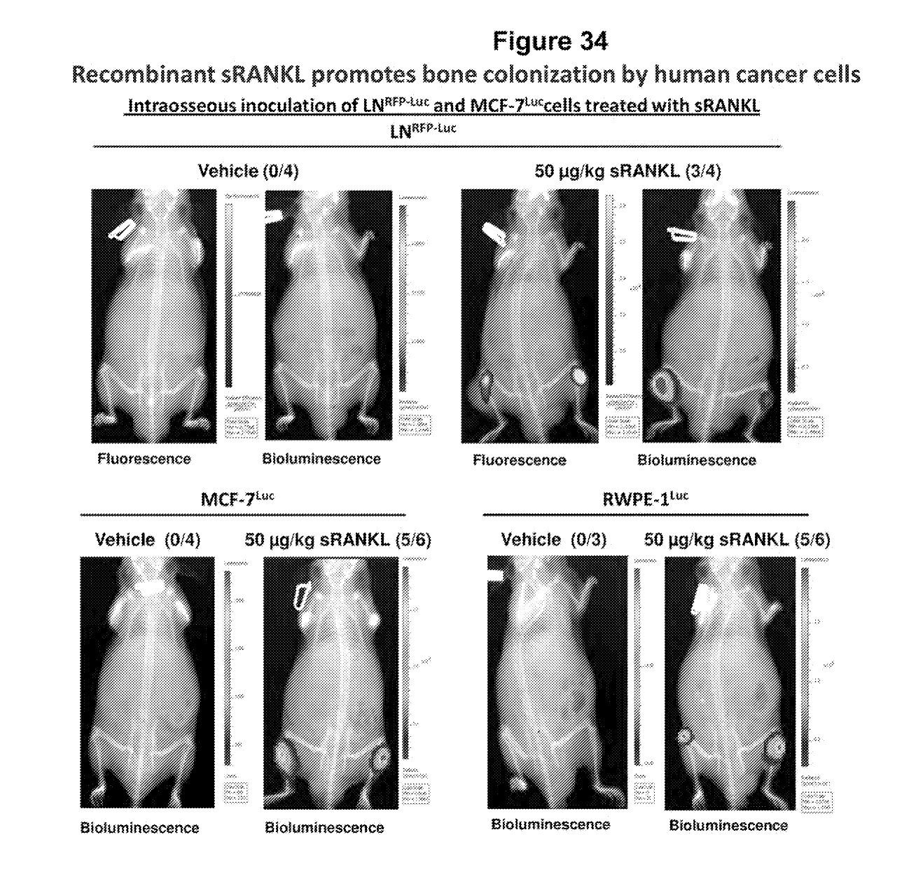

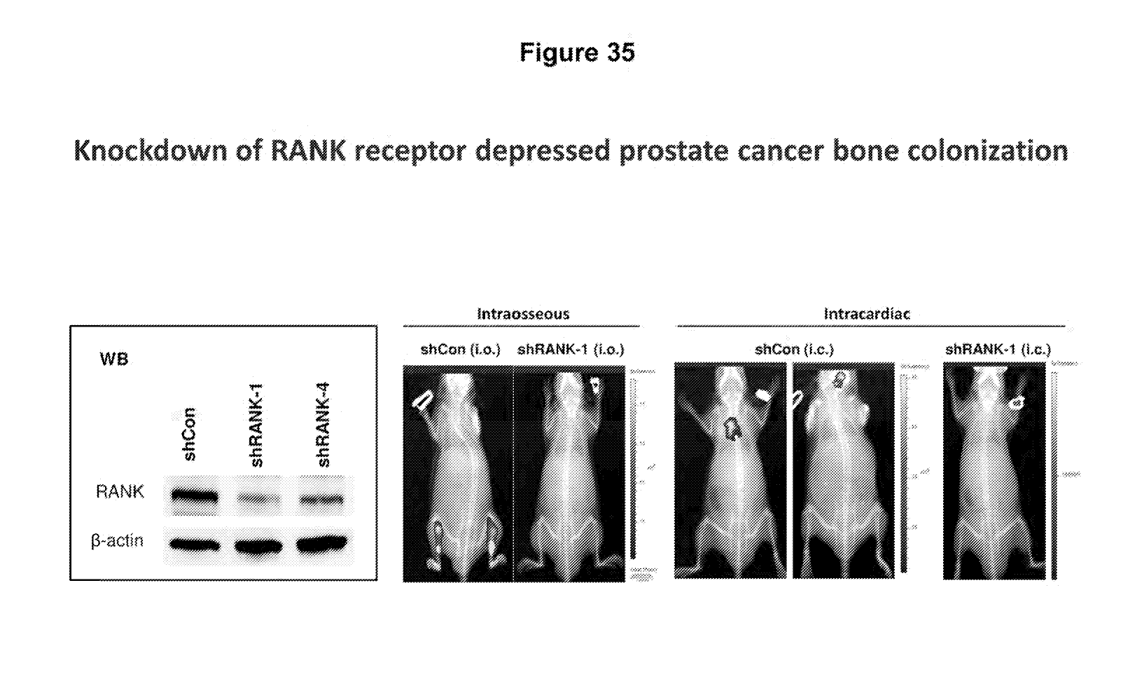

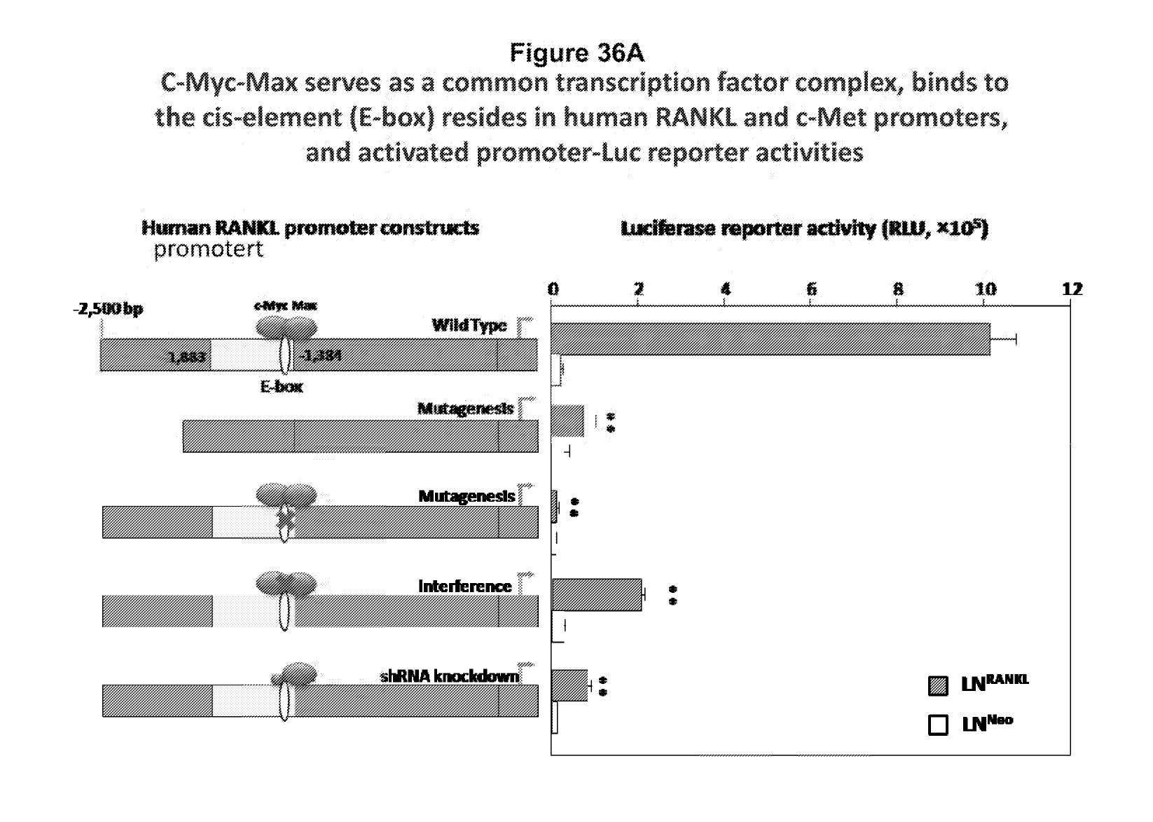

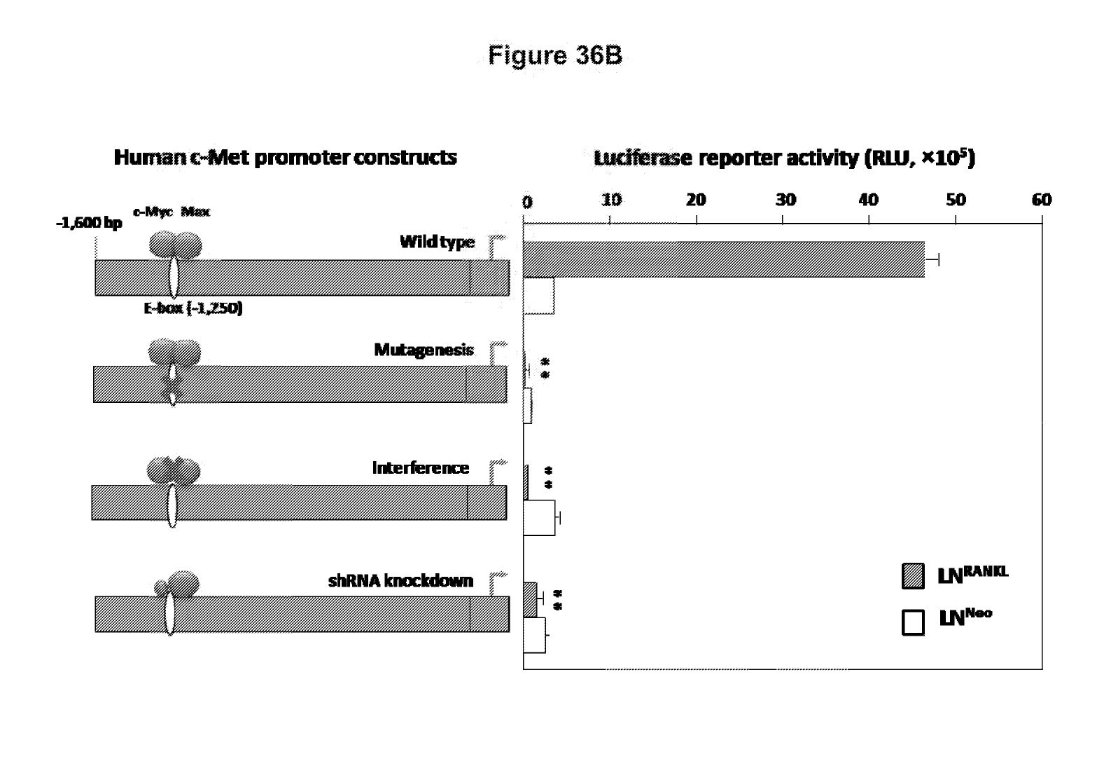

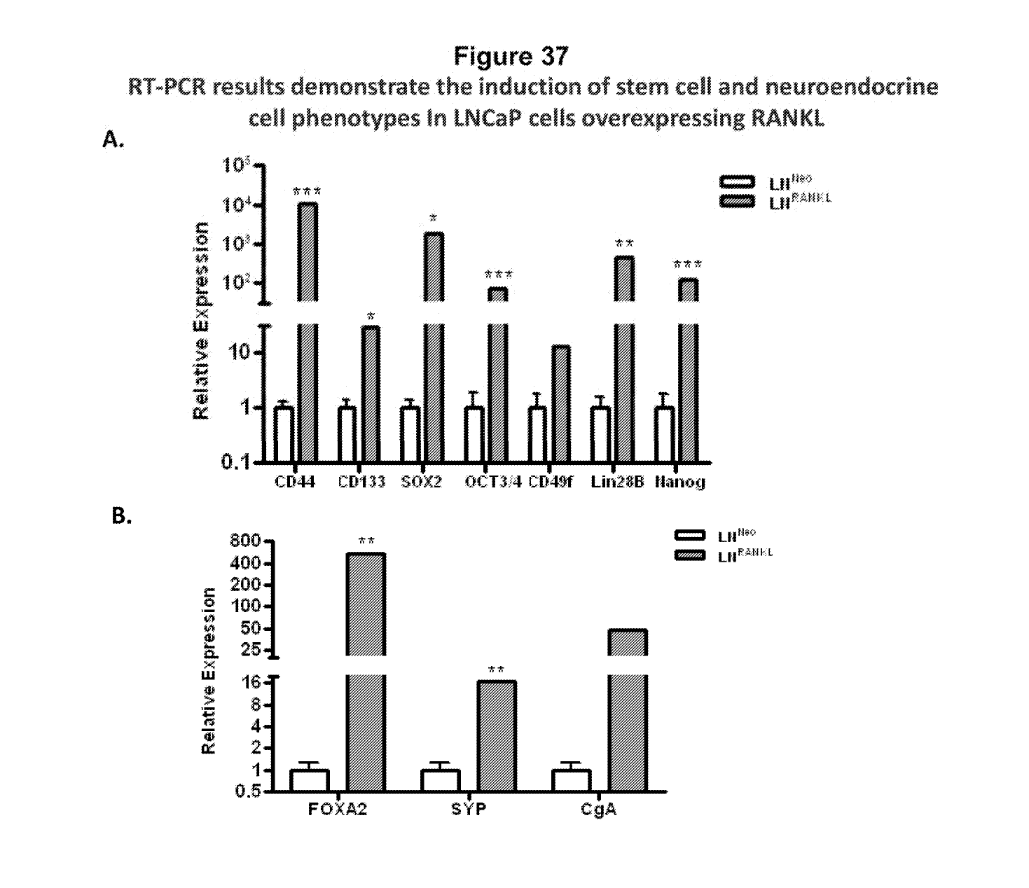

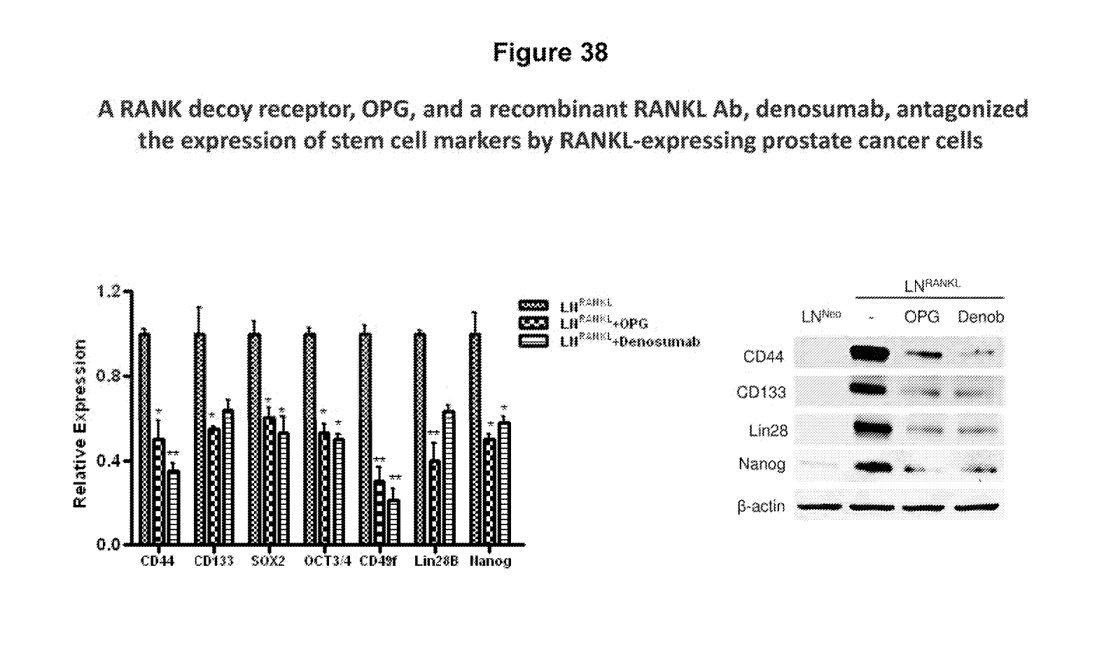

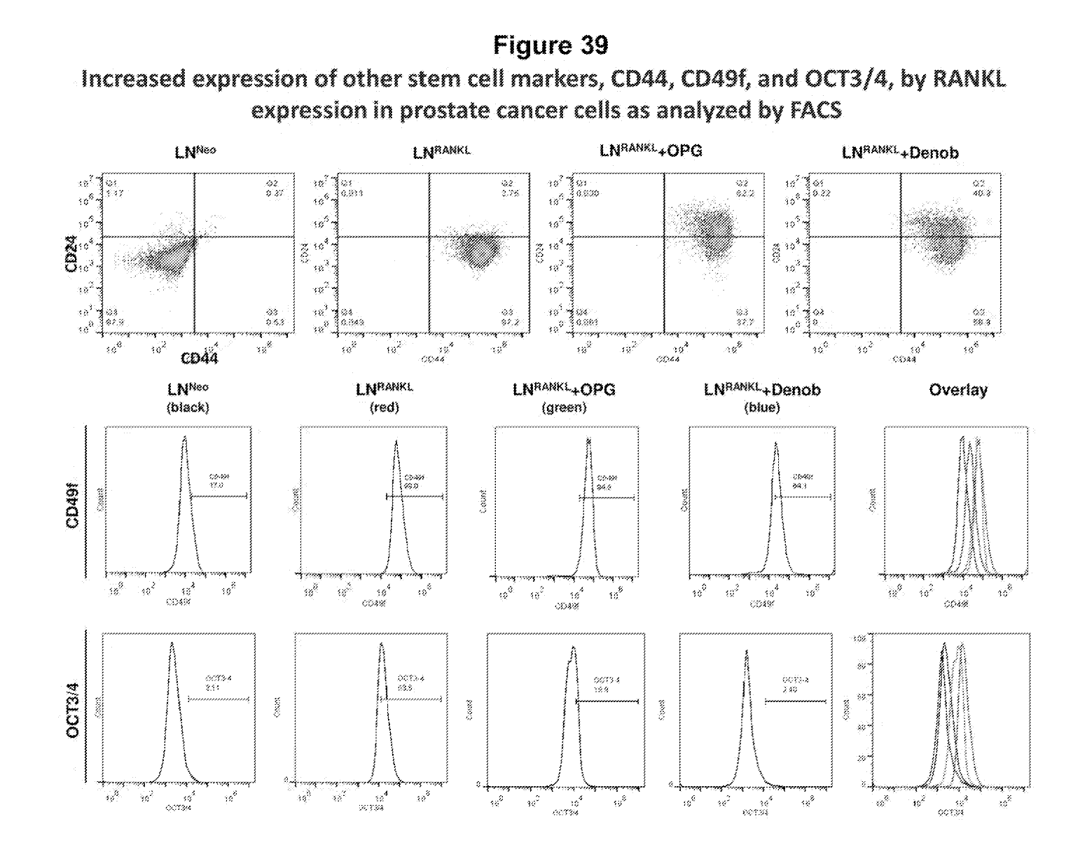

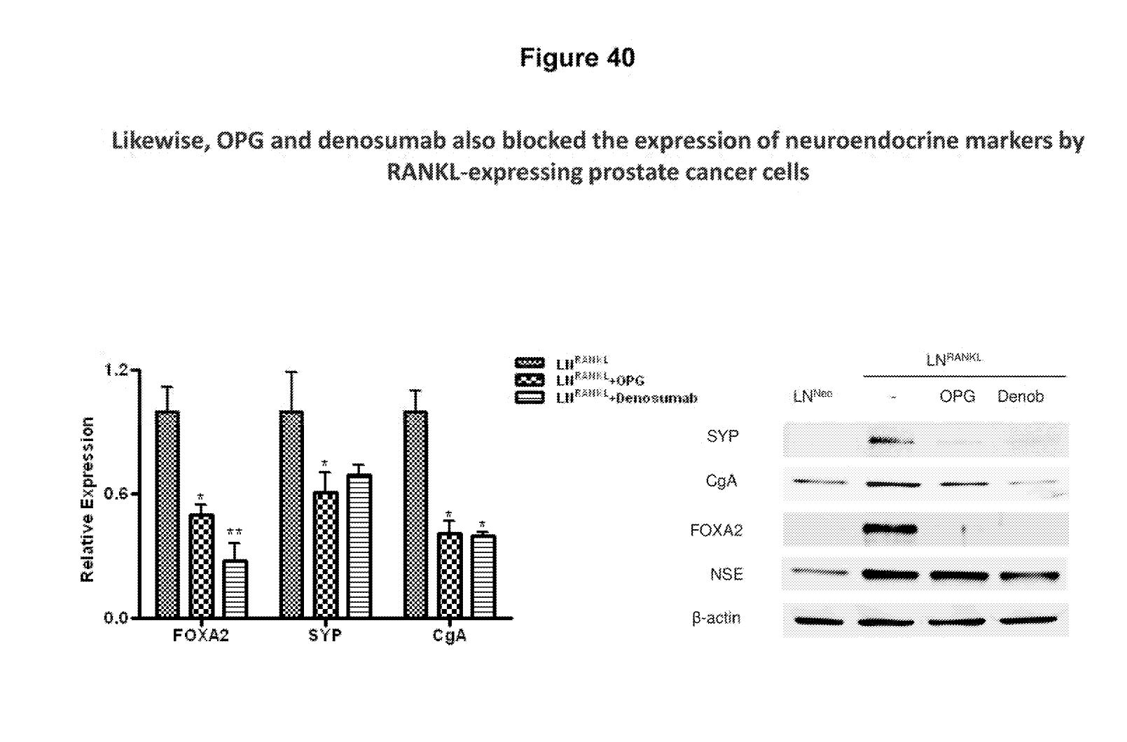

The inventors found that although LN^{RFP} cells were non-tumorigenic and non-metastatic, they could cohabit in bone and soft tissues with LN-RANKL cells. LN^{RFP} cells were detected in tumor specimens and cells derived from the tumors by fluorescence imaging (FIG. 24). Thus LN^{RFP} cells, considered as cancer cells in a dormant state, can be activated by the presence of aggressive LN-RANKL cells and become capable of co-colonizing metastatic bone and soft tissue sites (FIG. 24). This example illustrated the targeting RANKL, c-Met including but not limited to downstream activation of neuropilin-1, Src-kinase, Stat3, Mcl-1, and NF-kB could prevent the activation of tumor dormancy.

### Example 20

### Determination of the Biology and Functional Roles of RANKL and HGF Axes in Promoting PCa Interaction with Bone Cells and the Development of Invasive and Metastatic Disease

Conventional concepts depict the paracrine roles of OCG cytokines as promoting bone remodeling, angiogenesis [7, 9, 29-38], and the expansion of myoepithelial and stem cells in hormone-responsive organs [39-43]. Without wishing to be bound by any particular theory, the inventors believe that PCa cells with an osteomimetic phenotype, expressing bone markers, RANKL, RANK, OPG and M-CSF, hijack normal bone remodeling, and angiogenesis by activating converging RANKL/RANK- and HGF/c-Met-signaling axes, undergo EMT and participate directly in bone and soft tissue colonization. This concept is supported by clinical observations that both primary and bone metastatic PCa tissues express osteomimetic and EMT-associated biomarkers [15, 16, 19, 21]. Studying the autocrine/paracrine roles of RANKL, the inventors observed that enforced expression of RANKL in PCa cells promotes EMT and distant metastases to bone and soft tissues. LN-RANKL cells also showed increased c-Met expression, raising the possibility of a converging RANKL-RANK and HGF-c-Met cell signaling network leading to enhanced c-Met phosphorylation and downstream signaling including but not limited to downstream activation of neuropilin-1, Src-kinase, Stat3, Mcl-1, and NF-kB, which was confirmed in the LTL313 CRPC animal model and clinical specimens.

### Example 21

### Determination on Whether RANKL-RANK Autocrine Interaction Alone is Sufficient to Support PCa Bone and Soft Tissue Colonization

RANKL expression in LNCaP, C4-2 and ARCaP cells was shown to promote EMT and cancer cell migration, invasion and metastasis and these cells have also been shown to express RANK and OPG [46-48]. Since RANKL and RANK are expressed by bone cells and inflammatory cells, it is possible that in vivo both paracrine and autocrine RANKL-RANK interactions are crucial for PCa cells to develop bone metastasis. On the other hand, the inventors' studies have shown that autocrine RANKL-RANK interactions among PCa cells alone are sufficient for PCa cells to colonize the bone, suggesting that new therapies may focus on cancer cell-derived RANKL, which could bypass the undesirable side effects of osteonecrosis, reported in some patients treated with bisphosphonates and denosumab [49, 50]. To develop a cancer cell-derived inhibitor of RANKL, it is conceivable to concentrate on the structure of RANKL on tumor cell surface and its regulatory mechanisms of RANKL shedding by specific forms of proteolytic enzymes.

The inventors knockdown or knockout (KO) RANKL from OBs and/or RANK from OCs, and then compare the growth and bone colonization of LN-RANKL cells in host mice. RANKL/RANK in OBs/OCs is downregulated by lentiviral vectors established in the inventors laboratory that achieve >80% gene knockdown efficiency [51]. In brief, bone marrow, harvested from Balb/c mice, are infected with a lentiviral vector carrying a collagen 1 α2-RANKL shRNA targeting OBs, or a LysM-RANK shRNA targeting OCs (controls infected with vector constructs without an insert). The promoter specificity has been reported [49, 52] and confirmed in Dr. Bhowmick's lab [53], in which three transgenes, a tamoxifen-inducible collagen 1 α2-Cre-ER, a conditional stromal Tgfbr2^{floxE2/floxE2}, and Rosa26, were expressed in mice to study the roles of Tgfbr2 signaling in OBs [53-55]. Likewise, crossing Tgfbr2^{floxE2/floxE2} and LysM-Cre resulted in Tgfbr2^{LysMKO} mice, which showed greatly decreased OCs as detected by reduced TRAP⁺ staining and increased bone volumes. The cell-type specific deletion of these genes is controlled by the promoters, and the effectiveness of the deletion will be confirmed by qRT-PCR and western blots. These approaches are currently under investigation. In brief, Balb/c/nu host are lethally irradiated. They receive bone marrow transplantation with genetically engineered bone marrow cells via IV infusion. Four groups of mice (N=15/group) receive control, OB^{Collα2KO}, OC^{LysMKO} and both. Mice are allowed to recover for 3 wks and are then inoculated with LN-RANKL cells (5×10⁵ cells) by IC injection. Serum PSA is monitored every two weeks for a total of 8 weeks. Tumor growth is monitored by a NIR organic dye MHI-148, luciferase detection of tagged LN-RANKL cells, and X-ray or Tc⁹⁹ imaging for bone lesions indicative of cancer growth in bone [22, 56-59]. Tumor number, size and location are monitored biweekly for an 8-wk period. Mice are sacrificed and tumor presence is confirmed by H/E, IHC and western blots (p-c-Met, pNF-kB, p-Akt) and histomorphometric analyses (tumor/bone ratio). Mouse sera are analyzed for biomarkers.

While not wishing to be bound by any particular theory, the inventors believe that RANKL/RANK KO from OBs/OCs could reduce the number and size of LN-RANKL tumors in the mouse skeleton and soft tissues. This suggests: 1) Endogenous OBs/OCs interaction with PCa cells creates a favorable microenvironment for the growth, survival and colonization of PCa in bone. 2) Soft tissue metastases can originate from the skeleton, either because of direct effects on the bone microenvironment (lack of RANKL/RANK on OBs/OCs), on the evolution of PCa cells residing in the skeleton, or deficiency of tumor-promoting MSC and inflammatory cell recruitment due to the altered bone microenvironment. IHC confirms the specificity and extent of KO of RANKL/RANK in bone cells and decreased activation of c-Met, Akt and NF-kB signaling. Histomorphometric analyses helps to distinguish osteolytic and osteoblastic responses of bone to the invading PCa cells. PCa bone metastasis may be developed preferentially in regions where RANKL/RANK is expressed, with the presence of more TRAP+ cells. This can increase the local concentration of growth factors as a result of increased bone turnover elicited by OB^{RANKL}-OC^{RANK} and/or PCa^{RANKL}-OC^{RANK} interactions favoring PCa bone colonization. If this strategy does not affect the latency and incidence of PCa bone and soft tissue metastases it suggests the autocrine/intracrine RANKL-RANK pathway within PCa cells is dominant. This can affect the future development of new therapeutics targeting the interface of RANKL-RANK interactions in PCa cells to treat PCa metastases.

Gene KO using lentiviral vectors in bone marrow preparations could be incomplete, compromising the extent of PCa cells homing to bone and soft tissues. Since RANKL increases with aging, an alternative could be to test if LN-RANKL may be more metastatic in aged than in young mice. To avoid cell loss or transformation during in vitro lentiviral transfection to cultured cells, lentiviruses are delivered directly by intraosseous administration. Since RANKL could also be produced by other host cell types such as MSC, T- and B-cells, a lentiviral targeting vector driven by a ubiquitous promoter, CMV, is prepared to KO RANKL from multiple cell types. This approach could also help address the differential lethal irradiation of bone marrow, in which OCs are more effectively eliminated by radiation than OBs; the residual MSC can contribute to host-derived OBs obviating the expected biological effects of transplanted OBs. To overcome this problem, in vitro co-culture studies can be made: LN or LN-RANKL cells (tagged with RFP) are co-cultured with purified OBs from neonatal mouse calvaria and/or OCs from mouse bone marrow macrophages, tagged with GFP using established methods [60-63]. The growth of PCa^{RFP}, OCs^{GFP}, and/or untagged OBs is monitored by FACS and confocal microscopy. In some of the co-culture studies, the effects of RANKL/RANK KO OBs/OCs are used to ascertain the functional roles of the RANKL axis between PCa and OBs/OCs.

The advantages of the co-culture studies are: 1) they are highly versatile, so mouse cells can be replaced by human OCs and OBs; 2) the co-culture can be performed in a 3-D system using either RWV or Hydrogel [64, 65]; 3) co-culture can be performed on bovine bone to assess osteoclastogenesis, angiogenesis, and mineralization of bone; 4) co-culture can allow measurement of cell proliferation (by measuring RFP or GFP-tagged cells), differentiation and survival (using appropriately engineered promoter reporter constructs such as collagen 1 α2-luc to measure OBs differentiation, Mcl-1-luc to measure cell survival, osteocalcin-luc to measure PCa osteomimicry and OBs differentiation and TRAP⁺ for OCs differentiation) [15, 28, 66, 67].

Conditional OC RANK KO mice generated by crossing RANK^{flox/flox} and LysM-Cre mice are established. The resulting OC^{RANK-/-} mice will be bred into the Rag^{-/-} background. The mice are expected to exhibit complete RANK deletion from OCs, allowing the observation of PCa growth and metastases to bone and soft tissues in the absence of host OC RANK activity. Control studies will be conducted using mice with the same genetic background but with intact RANK in OCs. Similar studies to generate mice with RANKL KO in OBs are not feasible at this time because of the lack of a RANKL^{flox/flox} strain.

### Example 22

### Determination on Whether the HGF-c-Met Signaling Axis Establishes “Cross-Talk” with RANKL-RANK Signaling Axis

The inventors determine if increased c-Met expression in PCa cells, in response to increased RANKL-RANK signaling, could enhance HGF-c-Met autocrine/paracrine signaling and result in increased PCa bone metastasis; and if increased HGF-c-Met signaling in PCa cells could enhance their response to RANKL-induced osteoclastogenesis and bone colonization. Since there is significant divergence between human and mouse HGF, the inventors chose to investigate the functional roles of human HGF (hHGF) in the context of human PCa metastases in two models.

First Model,

HGF-Rag^{-/-} transgenic mouse model established in Dr. George Vande Woude's laboratory were used. They were inoculated with 5×10⁵ cells IC/mouse, 15 mice/group, of LN-RANKL (which as expected, responded to HGF). They are also inoculated with 5×10⁵ cells IC/mouse, 15 mice/group, with c-Met KO (it is expected to not respond to HGF). The growth and metastatic potential of the cells and signal activation are measured; control experiments are conducted by injecting the cells IC in Rag^{-/-} transgenic mice not expressing hHGF, not expecting to see differences of tumor metastases of these cells. Plasma concentrations of hHGF in mice are determined by ELISA. hHGF levels correlates with the incidence, latency, size and location of PCa metastases. hHGF and c-Met phosphorylation in PCa tumors harvested from different metastatic sites are determined. Mcl-1, a downstream anti-apoptotic target of c-Met, Src-kinase and Stat3 phosphorylation, mediators of Mcl-1, are measured and used as read-outs [28]. In addition, a c-Met “activation signature” is measured, which was identified in hepatocellular carcinoma [68] and validated in breast cancer [69] in PCa cells.

Second Model,

HGF-Rag^{-/-} and control Rag^{-/-} transgenic mice (15 mice/group) are inoculated with ARCaP_{E} cells which express endogenously c-Met, RANK and RANKL. Since ARCaP_{E} cells are marginally metastatic, it is determined if endogenous hHGF in HGF-Rag^{-/-} mice may activate HGF-c-Met signaling and increase PCa bone colonization. The growth and metastasis of PCa tumors in mice are evaluated. The status of RANKL-RANK signaling in the harvested tumors is assessed by the determination of the phosphorylation of p38 MAPK, PI3K-Akt and NF-kB (FIG. 25). It is to be tested whether HGF-c-Met signaling axis activates or “cross-talks” with RANKL-RANK signaling axis.

Expected Results and Data Interpretation:

LN-RANKL cells are more metastatic than LN-RANKL/c-Met KO in colonizing the bone and soft tissues in HGF-Rag^{-/-} mice. Without wishing to be bound by any particular theory, the inventors believe that this homing ability will correlate with c-Met phosphorylation in PCa cells. If c-Met KO in LN-RANKL cells does not affect the incidence, latency and/or pattern of metastases, this suggests the dominant role of RANKL-RANK signaling in determining LN-RANKL metastases. Serum and tissue hHGF levels, and c-Met phosphorylation in PCa and OCs may correlate with and predict the ability and the site of PCa metastases in mice. Without wishing to be bound by any particular theory, the inventors believe that cells with activated HGF- and RANKL-signaling axes will also exhibit increased Mcl-1 expression and elevated Src-kinase and Stat3 phosphorylation with increased c-Met “activation signature”.

Alternatives:

The current study focuses on the activated forms of HGF and RANKL, which under pathophysiological conditions are regulated by proteolytic cleavage enzymes (see FIG. 21). These enzymes are responsible for the release of these cytokines in a context-dependent manner [70-75]. The growth, survival and expansion of metastatic tumors also require angiogenesis under the control not only of the RANKL- and HGF-axes [76-80] but also the VEGF- and TGFβ-signaling axes. As an alternative experimental approach, co-culture studies are conducted using LN-RANKL^{RFP}/LN-RANKL^{RFP-MetKD} cells (i.e., without/with c-Met knockdown), and ARCaP_{M}^{RFP-RANKL-high} and ARCaP_{E}^{RFP-RANKL-low} (ARCaP cells expressed intrinsically high or low RANKL) cultured with OBs^{GFP}/OCs^{GFP} cells. The growth of the mixed populations is assessed by FACS and confocal analyses. The expression of anti-apoptotic gene Mcl-1 and the status of differentiation of OCs-TRAP⁺ and OBs (osteocalcin⁺ and Runx2⁺) cells in the co-culture are assessed [28]. Finally, PCa growth is established in bovine bone to determine the functions of OCG cytokines in angiogenesis, osteoclastogenesis, osteoblastogenesis and bone mineralization. Shedding of OCG cytokines by proteolytic cleavage could be important. A study to assess the role of MT1-MMP in the tumor microenvironment is performed by comparing the amount (ELISA) and the activity of sRANKL on OCs (TRAP⁺) in co-culture of PCa with calvarial bone obtained from MT1-MMP^{-/-} mouse [81]. It has been reported that calvaria from MP9^{-/-} mice were unable to activate/cleavage pro-angiogenic cytokines and failed to undergo osteoclastogenesis[82].

### Example 23

### The Transcription Factors Activated by RANKL-RANK Interaction Autoregulate RANKL Expression in PCa Cells

Since RANKL expression drives PCa bone metastasis, understanding how the autoregulation of RANKL is executed could help devise strategies to override the program and to block PCa bone metastasis. Two transcription factors (TFs), CREB and c-Myc, were identified as essential for the RANKL positive feedback loop in PCa cells. These TFs are known to control a host of other important regulatory genes that affect the global pathophysiology of cancer cells [83, 84]. For example, the participation by CREB in RANKL transcription could also lead to increased SREBP-1 [85], a TF known to regulate androgen receptor (AR). SREBP-1, when expressed in PCa cells, upregulates not only AR but also fatty acid synthase and Nox 5, and downregulates catalase leading to an accumulation of lipid droplets and hydrogen peroxide in PCa cells (FIG. 22). These factors are known to promote PCa progression [86]. In LN-RANKL cells, elevated levels of SREBP-1, fatty acid synthase, Nox 5 and ROS were found but not AR (data not shown), supporting the regulatory role of SREBP-1. These findings suggest that coordinated upregulation of RANKL in PCa cells induces a metabolic switch of PCa cells toward increased accumulation of lipids through enhanced lipogenesis and ROS. Activation of c-Myc has been shown to cooperate with the HGF-c-Met axis, which can activate a downstream effector, Snail, shown by the inventors' laboratory to promote EMT and cancer metastasis [18, 19, 21], with a concomitant increase in intracellular ROS [3]. These results, taken together, support the importance of the converging RANKL- and HGF-signaling axes for inducing ROS and lipogenesis in PCa cells to promote PTEN inactivation and stem cell renewal. To confirm that these TFs bind to their respective specific cis-elements, perform gel shift, supershift, chromatin IP and promoter site-directed mutagenesis are performed as reported by the inventors' laboratory [87-90]. Moreover, whether the antioxidant N-acetyl-L-csyteine (NAC) inhibits the growth of LN-RANKL cells by lowering oxidative stress, by antagonizing NF-kB activation, a downstream target of the RANKL- and HGF-signaling axes, are tested using the inventors' groups' published protocols [3, 18, 64, 86].

Expected Results and Data Interpretation:

Without wishing to be bound by any particular theory, the inventors believe that the action of CREB and c-Myc in the regulation of RANKL transcription can be confirmed by these studies. These TFs could act as a complex by forming heterodimers with other TFs. Although RANKL is expressed not only by cancer cells but also by host inflammatory cells, the positive feedback loop of RANKL on RANKL expression will be cancer-specific and might be explained by differential TF activation. These results are supported by recent reports where three human PCa cell lines showed upregulated (2.5- to 4-fold) cell surface RANKL when in close contact with osteoblasts (which express RANKL) in co-culture [91-93]. This observation could have the following important biologic implications: 1) PCa cells can serve as amplifiers for RANKL production. The production of RANKL by a few cancer cells or inflammatory cells in the cancer cell neighborhood can conceivably stimulate RANKL expression by PCa cells, by either membrane bound RANKL or sRANKL shedding from cell membranes by proteases (e.g. MT1-MMP) [81, 94, 95]. 2) Activation of the RANKL axis promotes c-Met expression, creating a converging signaling axis with HGF-c-Met, and triggering downstream activation of common signaling pathway components shared between RANKL and HGF axes, such as p38, PI3K-Akt and NF-kB (FIG. 25), supporting increased PCa cell proliferation, survival, angiogenesis and osteoclastogenesis, ultimately contributing to PCa bone colonization. 3) PCa cells expressing RANKL could conceivably awaken other dormant cancer cells in bone to express RANKL and increase osteoclastogenesis and angiogenesis, further promoting cancer cell colonization of the skeleton. Alternatively, the RANKL expressing PCa cells could create a fertile microenvironment through increased interactions with OCs and OBs facilitating skeletal colonization by PCa cells irrespective of their basal levels of RANKL expression. 4) RANKL expressing cells were observed to undergo a metabolic switch toward lipid accumulation, enhanced ROS and increased AR activity. This switch can enhance CRPC transition with the potential for increased genomic instability. Secreted ROS can induce increased oxidative stress and expansion of the stem cell niche. 5) Elevated c-Myc could increase Src kinase, a known downstream signaling node of the HGF-axis, and also an attractive target for PCa therapy [96-99].

Alternatives:

Whether RANKL stimulates RANKL expression in an autocrine/paracrine manner in lung, breast, renal cancer and multiple myeloma cells, known to metastasize to the skeleton, is examined.

### Example 24

### To Target the Converging RANKL- and HGF-Signaling Axes and Determine the Effectiveness of Co-Targeting these Pathways and their Downstream Effectors Using Cell Culture and Co-Culture Models

Data collected from laboratory and clinical studies suggest targeting the converging RANKL- and HGF-signaling axes separately is clinically effective. To improve the targeting potential of these converging signaling mechanisms, the inventors propose to target sequentially four interrelated cell signaling networks: RANKL-RANK, HGF-c-Met, N-cad and oxidative stress. The rationale of this approach is that blockade of RANKL- and HGF-signaling axes is expected to reduce but not eliminate EMT-, N-cad- and oxidative stress-induced PCa cell migration and invasion. Targeting N-cad and oxidative stress could further attenuate the progression of PCa cells that escape blockade of the RANKL- and HGF-signaling axes [45]. This approach could also blunt other GF-, VEGF-, ECM- and MMP-mediated increases of PCa EMT, and associated cell migration, invasion and metastases. Many of these signaling pathways are known to converge via increasing N-cad and accumulation of intracellular ROS and lipogenesis[100-102]. The basic experimental design is: LN^{RFP}, LN-RANKL^{RFP} or ARCaP_{M}^{RFP-RANKL-high} cells cultured either alone or together with freshly prepared mouse calvarial OBs^{GFP}/marrow macrophage-derived OCs^{GFP}, with or without the presence of the bovine bone. The cells are exposed to individual targeting antibody or small molecules, in a dose-response manner, directed toward RANKL (denosumab), HGF (AMG102 or XL-184), N-cad (1H7) and/or ROS (NAC). Cell growth will be monitored by FACS sorting and validated by confocal microscopy. While not wishing to be bound by any particular theory, the inventors believe that denosumab+XL-184 (use IC₅₀) can additively block the growth of PCa cells and interrupt their interactions with OBs/OCs. The growth and differentiation of the bone cells are assessed as described above. If PCa cells resist blockade of RANKL and HGF signaling, N-cad and ROS as the blocking agents are to restore the growth inhibitory mechanisms. These targeting experiments are conducted in the presence or absence of soluble human HGF and/or human RANKL, to obtain basal and activated states of the cell signaling network. To assure that the results can be applied in the clinic, the study is expanded to include other human CRPC cells known to metastasize to bone (C4-2 and PC-3). The most effective targeting combinations will be selected for in vivo studies.

Expected Results and Data Interpretation:

Combined targeting should be the most effective and durable because some PCa cells escape the targeting of single signaling axis. All the reagents selected for targeting are considered relatively non-toxic in the treated hosts, which will increase the likelihood of observing the effectiveness of these combined agents and facilitate clinical translation. This combination strategy could lower the dose of drugs needed to control tumor growth and metastasis and thus reduce systemic toxicity. This targeting strategy will result in reduced growth and enhanced differentiation of OCs (TRAP⁺), OBs, and PCa cells in co-culture and cancer-induced osteoclastogenic and angiogenic responses in bone explants.

### Example 25

### To Develop Novel Co-Targeting Strategies Using Human PCa Xenograft and Bone Metastic Models

A human LTL-313 xenograft model that mimics CRPC in PCa patients was established by Dr. Y. Z. Wang. A chimeric LN-RANKL/LN bone metastasis model that mimics PCa in patients is chosen for the study of mixed osteoblastic and osteolytic responses in bone. These models are used for both prevention (initiate drug treatment 2-wks after castration) and tumor reduction (initiate drug treatment after tumor has been established by imaging criteria in castrated hosts). Intact male Balb/c/nu mice will be implanted with LTL-313 tumors orthotopically for localized CRPC. For bone metastasis, a chimeric osteoblastic model by injecting a mixture of LN-RANKL:LN^{RFP} (1:9) cells (5×10⁵/mouse) IC is established. One week after tumor induction, mice are castrated, wait for 2-wks, to establish CRPC. For prevention and tumor reduction studies, a combination therapy is used to save the total number of mice used. A total of 60 Balb/c/nu are randomized for either the prevention or tumor reduction study with 30 mice/group. They are randomized (15 mice/group) to receive either vehicle or sequentially treated with denosumab mAb and XL-184 to control tumor growth and serum PSA elevation. If serum PSA relapses, they are then treated with combinations of a N-cad mAb, 1H7, and an antioxidant, NAC. Tumor growth is followed by NIR imaging with MHI-148 or luciferase bioluminescence imaging as reported by the inventors' group [56-59]. For bone metastasis, X-ray and/or Tc⁹⁹ bone scan are used [22]. Changes in incidence, tumor size and location of the tumors in the hosts are monitored every two weeks for a total of 8 weeks. When the experiments are terminated, mouse tumor tissues and metastases are harvested for H/E, histomorphometry and IHC analyses of the tissue sections. Mouse serum and marrow contents are harvested for biomarker analyses and comparison between the targeted and control vehicle treated groups.

Expected Results and Data Interpretation:

Without wishing to be bound by any particular theory, the inventors believe that the combined targeting strategy will be most effective against the growth of freshly established CRPC PCa tumors and bone and soft tissue metastases in the experimental prevention models. The responsiveness of established PCa tumors in both CRPC and metastasis models in the tumor reduction studies could be less, and require longer treatment time and higher dose to observe tumor regression. PCa tumor response to therapy is recognized by NIR imaging (recognizing both soft tissue and bone metastases), and luciferase imaging (with better penetration than NIR agents for deep spine metastases (data not shown). Bone metastasis can also be detected by X-ray and Tc⁹⁹ bone scan. As tumor growth at the primary or metastatic sites responds to therapy, the inventors expect a sharp drop of serum PSA with objective tumor volume reduction detected by tumor images and supported by a host of predictive serum biomarkers originating from bone and tumors. The inventors expect reduced levels of these markers in serum and tissues, and the decline of these markers will be correlated with serum PSA decline. The responsiveness of the serum and tissue biomarkers will correlate to a large extent with the tumor volume, serum PSA and the induction of apoptosis and blockade of angiogenesis as previously demonstrated by the inventors' laboratory [25, 57, 106-110].

Alternatives:

Additional animal studies are performed using an additional 150 mice to test the effectiveness of the individual agents in different combinations to understand the targeting mechanisms of the OCG cytokines and ROS with target-specific antibodies and antioxidant, and the roles of VEGFR2, c-Ret, c-Met in the target models. These additional studies could help resolve potential questions of the roles of multiple tyrosine kinases as potential targets for XL-184, the antibody competition. The PK/PD profiles of the therapeutic agents could determine the proper scheduling of these targeting agents. If LTL-313 has a slower growth rate in castrated mice, androgen-independent LTL-220M line is used, which grow more robustly in castrated hosts as a replacement.

### Example 26

### To Conduct Pilot Studies to Test the Utility of Potential PCa-Derived Factors in Clinical PCa Serum, Tissue and Marrow Biopsy Specimens

PCa bone metastasis is predominantly osteoblastic in contrast to the osteolytic pattern seen in breast cancer. Clinical assessments of the growth of PCa tumor in the skeleton and therapeutic responses are restricted to markers related to bone cells and bone turnover such as alkaline phosphatase, N-terminal collagen 1 C-telopeptide (N-telopeptide), osteocalcin, cross-linked pyridinoline and deoxypyridinoline (D-PYD) with results validated by MRI, X-ray and T⁹⁹ scintigraphy. Using the human LN-RANKL model, the inventors discovered five classes of factors that are produced by the metastatic PCa cells (Table 2) that could be used clinically for assessing therapeutic responses to PCa bone metastasis therapy. The inventors propose: 1) to conduct preclinical animal studies and characterize the secreted factors expressed by localized chimeric human LN-RANKL:LN tumors grown subcutaneously (a non-metastatic model with injection cell:Matrigel, 1:1 mix) (N=15) and compare the profiles of these secreted factors in chimeric LN-RANKL:LN tumors metastasizing to bone (N=15). In separate studies the inventors profile and compare the secreted factors in serum of mice bearing chimeric LN-RANKL:LN cells injected either subcutaneously or intraosseously (N=15 each). 2) to screen for the presence of secreted factors in human sera (N=60) collected from CRPC patients who have been subjected to neoadjuvant trials, but without radiographic evidence of bone metastasis, and compare the secreted factors against another group of CRPC patients who presented with radiographic evidence of bone metastases (N=60). Criteria are established to exclude secreted factors that might yield a high noise/signal ratios such as factors regulated by androgen or lack of androgen, factors that are involved in skeletal development and bone diseases, and factors related to chronic inflammation. The inventors propose to profile MCP-2, GRO, GCP-2, MMP8 and MMP10 which are believed to be representative of secreted factors from PCa bone metastasis. The expression of these proteins is assessed in bone marrow aspirates obtained from PCa patients with confirmed bone metastasis. For the preclinical animal studies, sera obtained from mice are subjected to either protein array or ELISA and results will be compared between localized versus bone metastatic PCa. The expression of secreted factors can be measured in as little as 1 μl of serum; hence the inventors can conduct a time-course study of these markers in live mice (e.g., detect marker changes every 2 wks for a total of 8 wks). To optimize the chances of success, the inventors conduct pilot studies evaluating PCa-derived factors in clinically defined specimens.

Expected Results and Data Interpretation:

While not wishing to be bound by any particular theory, the inventors expect some concordance of the secreted factors in mouse and human sera. Better concordance may be observed in secreted factors in human sera and bone marrow aspirates from men with confirmed PCa bone metastasis. Marked differences in both the type and level of secreted factors are expected to emerge in human sera from patients with and without confirmed bone metastasis. If secreted factors produced by osteomimetic PCa cells could also be produced by bone cells, these secreted factors are more likely to be expressed by PCa cells grown in bone than PCa cells grown locally. Like bone-derived factors that predict therapeutic responses, PCa-derived factors can also predict the progression and responsiveness of PCa patients to androgen deprivation or combination therapy. The responder patients can be expected to express lower levels of chemotaxis, osteoblastogenesis, osteoclastogenesis, proteolytic cleavage enzymes and angiogenesis markers than the non-responders. Because of the plasticity of cancer cells, markers associated with cancer cells may be more disease stage- and contact-dependent. Monitoring changes as a function of time will be valuable and could be used to evaluate risks.

Alternatives:

To increase the chances of identifying unknown protein factors produced by CRPC using the PCa model systems described above, two new approaches are used: 1) increased search for new cytokines with other commercially available antibody arrays, and 2) the use of advanced Isobaric Tags for Relative and Absolute Quantitation (iTRAQ) [113, 114] and mass spectrometry to detect new unknown protein factors produced by the cell models. The principle of the iTRAQ method is to tag the secreted proteins in the conditioned media harvested from cells cultured in different growth conditions. The protein factors in the conditioned media of LN or LN-RANKL cells, either with or without androgen (R1881) exposure, or disaggregated LTL131 cells from tumors growing in intact and castrated hosts, are harvested and enzymatically digested with trypsin, and labeled with one of the two iTRAQ reagents in pairs. Samples are then mixed and analyzed by tandem mass spectrometry (MS/MS). Each of four iTRAQ reagents increases the MW of the secreted proteins corresponding to each peptide, and the intensity of reporter ions can be analyzed with simultaneous sequencing and quantification of the labeled peptides. Other proteomics methods are available to us if necessary, including SILAC, a robust quantitative proteomics approach for measuring differences in protein levels in cultured cells and applicable on a proteome scale [41, 115].

### Example 27

### Cell Culture

Isogenic human prostate cancer LNCaP C4-2, and C4-2B, were established as previously described. PC-3, PC-3M and DU145 human prostate cancer cells and MG63 human osteosarcoma cells were obtained from ATCC (Manassas, Va.). LNCaP, C4-2, C4-2B, PC3, PC3-M, DU145, and MG63 were all maintained in RPMI 1640 supplemented with 5% FBS. ARCaP_{E} and ARCaP_{M} cells, established by the inventors' lab, were maintained in T-medium (Invitrogen, Carlsbad, Calif.) supplemented with 5% FBS. SaOS-2 and RAW264.7 cells (kindly provided by Dr. Neil Weitzmann, Emory University, Atlanta, Ga.) were maintained in DMEM supplemented in 10% FBS.

### Example 28

### Transfection

ARCaP_{E} or ARCaP_{M} cells were seeded into 6-well plate and transiently transfected with flag-tagged RANKL or neo plasmid DNA under the regulation by CMV-promoter, or RANKL siRNA (Santa Cruz Biotechnology, Inc., Santa Cruz, Calif.) with an universal scramble siRNA which serves as the control (Invitrogen, Carlsbad, Calif.). After 48 h of transfection, cells were harvested for western blotting and RT-PCR analyses. For stable transfection, LNCaP cells were first transfected with either flag-tagged RANKL- or neo-control-plasmid cDNA (Sigma-Aldrich, St. Louis, Mo.) in a 6-well plate for 48 h, and the cells were trypsinized and seeded into a 15 cm dish. The cells contain the transfected vector were selected in 400 μg/ml of G418 until individual colonies that contain transfected construct were formed. LNCaP cell colonies expressing flag-tagged RANKL or neo plasmid were picked and the cells were amplified from a 48-well plate to a 10 cm dish in the presence of G418. Stable clones were maintained in 200 μg/ml of G418 and routinely checked for flag-RANKL expression by western blot analysis.

ARCaP_{E}-RANKL and ARCaP_{E}-Neo stably transfected cell lines were established by a lenti-viral infection protocol. In brief, lenti-viral particles containing RANKL-GFP or GFP alone (Neo control) were produced by 293FT cells after transfection with Plvx-RANKL-AcGFP or Plvx-AcGFP-N1 plasmids (Clontech, Mountain View, Calif.) for 24 to 48 h. ARCaP_{E} cells were infected with the lenti-viruses containing DMEM media in 1 to 5 ratio in the presence of 8 μg/ml of polybrene (Sigma-Aldrich, Inc., St. Louis, Mo.) for 48 h, and the cells were selected, characterized and maintained in T Medium containing 4 μg/ml of puromycin.

### Example 29

### In Vitro Osteoclastogenesis Assay

The co-cultures of osteoclast precursor cells (RAW 264.7 cells) and prostate cancer cells were performed in 48-well plate containing 500 μl of α-MEM media supplemented with 10% FBS and were set up in quadruplicates. Osteoclast precursor cells were seeded with prostate cancer cells in an 8:1 ratio (15,000 RAW 264.7 cells and 1,875 prostate cancer cells) per well. Recombinant mouse RANKL (100 ng/ml, provided by Dr. Xu Feng), was added to the assay as the positive control. Additionally, recombinant OPG (1 μg/ml) was added to designated wells to block RANKL-mediated increased osteoclastogenesis. The cells were checked under the microscope daily from day-4 to day-7. Cells were fixed and subjected to tartrate-resistant acid phosphatase (TRAP) staining (Sigma-Aldrich, Inc., St. Louis, Mo.). TRAP⁺ multinucleated cells (3 nuclei) in the entire well were counted as mature osteoclasts and the images were taken under light microscopy.

### Example 30

### In Vitro Growth, Migration and Invasion Assays

RANKL- or Neo vector-expressing ARCaP_{E} and LNCaP cells were seeded into 96-well plate at 5,000 cells per well. For R1881 treatment, LNCaP-RANKL or LNCaP-Neo cells were serum starved in serum-free RPMI1640 overnight and treated in the presence or absence of 10 nM of R1881. Cell proliferation was examined daily by adding 20 μl of CellTiter 96 AQᵤₑₒᵤₛ One Solution Reagent (Promega, Madison, Wis.) into each well and incubating for 2 h. The plate was read at absorbance of 490 nm using a 96-well plate reader. The CellTiter 96 AQᵤₑₒᵤₛ One Solution Cell proliferation Assay is a colorimetric method capable of determining cell proliferation by assessing the total number of viable cells in each well.

Migration and invasion of cancer cells were examined in 24-well plates using either uncoated or collagen I- and growth factor reduced Matrigel (BD Biosciences, Bedford, Mass.)-coated transwells (8 μm pore size), respectively, as previously described. Cells were serum-starved in RPMI 1640 or T-medium (Invitrogen, Carlsbad, Calif.) overnight and dissociated in 10 mM EDTA/HEPES. The lifted cells were washed and counted, and 5×10⁴ cells were seeded in 100 μl of serum-free RPMI 1640 or T-medium in the upper chamber of the transwells in the presence or absence of 1 μg/ml OPG and 400 μl of RPMI 1640 or T-medium supplemented with 5% FBS was placed in the lower chamber of the transwells. After 24 h (migration) and 48 h (invasion) of culturing at 37° C., the migrated and invaded cells were fixed with 10% formaldehyde, stained with 0.5% crystal violet, and the cells at the upper side of the membrane of the chamber were removed and quantified.

### Example 31

### Reverse Transcription (RT)-Polymerase Chain Reaction (PCR)

Total RNA from cells was isolated using RNeasy Mini Kit (Qiagen, Valencia, Calif.) according to manufacturer's instructions. Complementary DNA (cDNA) was generated from 3 μg of total RNA using SuperScript® III First-Strand Synthesis System (Invitrogen, Carlsbad Calif.), and 1 μl of cDNA was subjected to PCR analyses using the following primers: RANKL F: 5′-TGG ATC ACA GCA CAT CAG AGC AG-3′ (SEQ ID NO:3); RANKL R: 5′-TGG GGC TCA ATC TAT ATC TCG AAC-3′ (SEQ ID NO:4); RANK F: 5′-GGG AAA GCA CTC ACA GCT AAT TTG-3′ (SEQ ID NO:5); RANK R: 5′-GCA CTG GCT TAA ACT GTC ATT CTC C-3′ (SEQ ID NO:6); OPG F: 5′-GCT AAC CTC ACC TTC GAG-3′ (SEQ ID NO:7); OPG R: 5′-TGA TTG GAC CTG GTT ACC-3′ (SEQ ID NO:8); E-cadherin F: 5′-GCC AAG CAG CAG TAC ATT CTA CAC G-3′ (SEQ ID NO:9); E-cadherin R: 5′-GCT GTT CTT CAC GTG CTC AAA ATC C-3′ (SEQ ID NO:10); N-cadherin F: 5′-GAT GTT GAG GTA CAG AAT CGT-3′ (SEQ ID NO:11); N-cadherin R: 5′-GGT CGG TCT GGA TGG CGA-3′ (SEQ ID NO:12); Snail F: 5′-CAG ACC CAC TCA GAT GTC AA-3′ (SEQ ID NO:13); Snail R: 5′-CAT AGT TAG TCA CAC CTC GT-3′ (SEQ ID NO:14); Vimentin F: 5′-GGA CTC GGT GGA CTT CTC-3′ (SEQ ID NO:15); Vimentin R: 5′-CGC ATC TCC TCC TCG TAG-3′ (SEQ ID NO:16). The PCR reaction cycles involved an initial denaturation at 94° C. for 10 min, followed by 36 cycles of 94° C., 1 min; 55° C., 30 sec for RANKL and RANK and 48° C., 1 min for OPG; 72° C., 1 min and a final extension at 72° C. for 10 min. For E-cadherin and N-cadherin gene amplification, the PCR reactions ran for a total of 32 cycles and the annealing temperature was 55° C. and 47° C., respectively for 30 sec. For Snail, Vimentin, and GAPDH amplification, the PCR reactions ran for a total of 28 cycles with annealing temperatures at 48° C. for 30 sec. The amplified PCR products were detected and analyzed on 1% agarose gel.

### Example 32

### Western Blot Analysis

ARCaP_{E} and LNCaP cells were cultured in 6-well plate and at 70% confluency, the cells were serum-starved overnight and then treated with 200 ng/ml of trimerized RANKL in serum-free and phenol-red free RPMI-1640 medium for three days before harvesting the cells. ARCaP_{E}-RANKL and LNCaP-RANKL cells were cultured in 6-well plate and treated with 20 μM of LY294002, a PI3K inhibitor, 20 μM of SB203580, an inhibitor of MAP kinase reactivating kinase, or 2 μM of PS341 (bortezomib), a proteasome inhibitor, at 90% confluency for four hours. Cells were lysed in RIPA buffer containing 1× protease inhibitor cocktail (Thermo Fisher Scientific, Waltham, Mass.) and centrifuged, and the supernatants were collected and quantified using Bradford Protein Assay (Thermo Fisher Scientific, Waltham, Mass.). Cell lysates (20-30 μg) were resolved on a 4-12% Bis-Tris gradient SDS-PAGE (Invitrogen, Carlsbad, Calif.) under reducing conditions, followed by transblotting onto nitrocellulose membrane (BioRad, Hercules, Calif.). The membranes were blocked in 5% non-fat milk in PBST for one hour at room temperature (RT) and incubated with diluted primary antibodies in blocking buffer at 4° C. overnight. The primary antibodies used were RANKL, E-cadherin, vimentin, and OPG (Santa Cruz Biotechnology, Inc., Santa Cruz, Calif.), RANK (Amgen, Thousand Oaks, Calif.), N-cadherin (BD Transduction Laboratories, San Jose, Calif.), phosphor-Akt (Ser 473), Akt, phosphor-p38 (Thr180/Tyr182), p38, phosphor-ERK1/2 (Thr202/Tyr204), ERK, phosphor-SAPK/JNK (Thr183/Tyr185), JNK, phosphor-NF-κB p65 (Ser536), and NF-κB p65 (Cell Signaling Technology, Danvers, Mass.). The membranes were washed with PBST for three times before incubating with peroxidase-conjugated anti-mouse or anti-rabbit secondary antibodies at RT for one hour. After three times washing, the membranes were visualized using ECL Plus reagent (GE Healthcare, Piscataway, N.J.).

### Example 33

### In Vivo Experiments

All animal procedures were performed according to approved protocol from the Institutional Animal Care and Use Committee. LNCaP-RANKL and LNCaP-Neo cells (1×10⁶ cells/50 μl PBS) were inoculated intracardially into 5- to 7-week-old male athymic nude mice (Charles River, Wilmington, Mass.), respectively using 28G1/2 needle (LNCaP-RANKL n=20; LNCaP-Neo n=15) as described previously. All mice were constantly monitored for metastatic tumor formation on a weekly basis by x-ray. Mice bearing metastatic bone tumors were first observed at 2 month, and mice were sacrificed at 3 month after injection. Before euthanasia, all mice were scanned with μCT tomography to examine the mouse skeleton and to determine the type of bone lesions. All metastatic tumors to bone or soft tissues were harvested and processed for histomorphological and histochemical analyses.

### Example 34

### Immunohistochemical Analysis

All reagents from the DAKO system were used for immunoperoxidase staining of section slides. Paraffin-embedded sections of bone and soft tissue tumors were rehydrated and antigenic epitopes were retrieved in citrate buffer using a pressure cooker. After antigen retrieval, slides were blocked with dual endogenous enzyme block (DEEB) at RT for 10 min and incubated with primary antibodies against Flag (Cell Signaling Technology, Danvers, Mass.), RANKL IMG-185A (Imgenex, San Diego, Calif.), RANKL NB100-56512 (Novus, Littleton, Colo.), RANKL FL-317 (Santa Cruz Biotechnology, Inc., Santa Cruz, Calif.), E-cadherin, N-cadherin, or vimentin (Santa Cruz Biotechnology, Inc., Santa Cruz, Calif.) at 4° C. overnight. The slides were placed at RT for 1 h and rinsed in Tris-buffered saline with 0.05% Tween (TBST) and incubated with Envision+Labeled Polymer-HRP at RT for 30 min. The slides were incubated with peroxidase substrate buffer with a chromogen, diaminobenzidine (DAB) to detect the staining signal, followed by hematoxylin counterstain of nuclei. After dehydration and cover-slipping, the slides were examined under light microscopy

### Example 35

### Tartrate-Resistant Acid Phosphatase Staining

The osteoclasts in tissue samples were detected by Tartrate-Resistant Acid Phosphatase (TRAP) staining. The bone tumors were harvested and decalcified in 10% ethylenediamine tetraacetic acid (EDTA)/PBS for 14 days and the decalcifying solution was changed every other day. The decalcified bone tissues were processed, paraffin embedded, and cut into sections. The bone tissue sections were deparaffinized and stained with the TRAP staining kit purchased from Sigma (St. Louis, Mo.) according to the manufacturer's instructions.

### Example 36

### RANKL Expression Correlates with Human Prostate Cancer Progression

The inventors examined RANKL expression in clinical prostate cancer tissue specimens of differing Gleason grade and tumor stage using an established IHC protocol. The inventors took special steps including the use of multiple anti-RANKL antibodies from commercial sources, immunoadsorption of these antibodies by recombinant RANKL protein, and validation of RANKL expression in experimental human prostate cancer cells and xonograft tissues by RT-PCR and western blots. Using the well-characterized anti-RANKL monoclonal antibody, the inventors assessed RANKL expression on one commercial TMA of benign and prostate cancer specimens and two custom-made TMAs containing well- and poorly-differentiated and bone metastatic prostate cancer specimens. The inventors observed low levels of staining for RANKL in benign prostate tumor (N=20) but positive staining in both the nucleus and cytoplasm in well- and poorly-differentiated human prostate cancer specimens (FIG. 6A, well differentiated N=18; poorly differentiated N=39). Significantly stronger immunostaining for RANKL was observed in the bone metastases (FIG. 6A N=43). Statistical analysis (one-way ANOVA) further confirmed the significantly higher expression of RANKL in the bone metastatic specimens over benign, well-differentiated (P<0.001), and poorly-differentiated prostate cancers (P=0.035) (FIG. 6A). RANKL expression in primary tumor was also higher compared to the benign tumor (P=0.023). These immunohistochemical results support a positive correlation between the level of RANKL expression and the progression of clinical prostate cancer specimens with increased RANKL expression from benign to locally invasive, and to skeletal metastases.

The inventors next examined RANKL, RANK, and OPG expression at both mRNA and protein levels in several pairs of isogenic human prostate cancer cell lines with differential propensity for bone and soft tissue metastases with two osteosarcoma cell lines, MG-63 and SaOS-2, served as positive controls. Differential expression at mRNA and protein of RANKL, RANK, and OPG was observed in each pair of these isogenic cell lines (FIGS. 6A and 6B). Positive correlations of RANKL expression and the aggressiveness of human prostate cancer cell lines were observed with higher expression in ARCaP_{M}, C4-2/C4-2B, and PC-3M cells than the corresponding parental, ARCaP_{E}, LNCaP, and PC-3 cells; higher levels of RANKL expression was found in more aggressive osteosarcoma MG-63 than the indolent SaOS-2 cells. RANKL receptor, RANK, was expressed by all of the prostate cancer cell lines, and strong expression was observed in ARCaP_{M} and PC3M cells. RANK mRNA expression is not always correlated with RANK protein expression in LNCaP cells. While an inverse relationship of protein expression was observed between RANKL and OPG, this was no correlation between RANKL and OPG mRNAs however. In the ARCaP EMT prostate cancer progression cell model, the inventors demonstrated consistently that the more invasive and metastatic ARCaP_{M} cells had stronger expression of the RANKL/RANK/OPG triad than the less invasive ARCaP_{E} cells. Among these isogenic prostate cancer cell line pairs, ARCaP_{E} and LNCaP cells showed the lowest expression level of the RANKL, RANK, and OPG. Collectively, these results indicated that RANKL, RANK, and OPG were expressed by most of the human prostate cancer cell lines the inventors examined, and their levels of expression generally correlated with the virulence of these cells.

### Example 37

### RANKL Induces EMT Progression in Human Prostate Cancer Cells

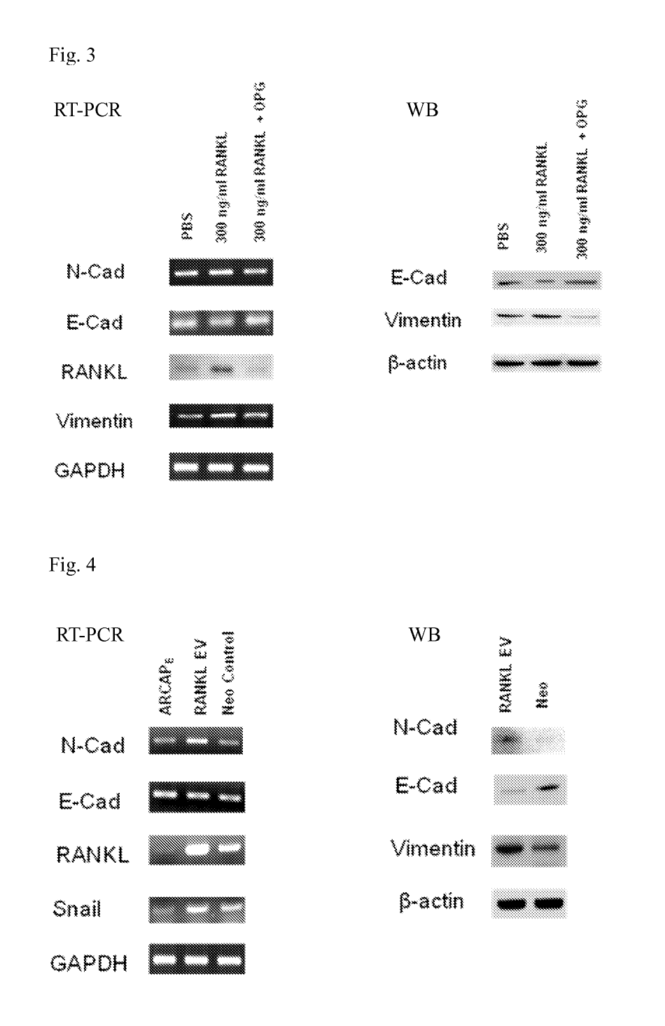

Given the inventors early observation that RANKL expression increased corresponding to EMT induced in ARCaPE cells upon addition of soluble growth factors here the inventors examined if exogenously added recombinant RANKL, or forced RANKL expression genetically in prostate cancer cells may contribute directly to their mesenchymal transition. The inventors used two cell models for this study, the ARCaP EMT cell model and the LNCaP cell model. The inventors' previous investigations revealed that ARCaP cells when provoked to assume EMT by soluble growth factors underwent morphologic, biochemical and behavioral changes, whereas the LNCaP cell model when provoked to undergo EMT by soluble growth factors only exhibited biochemical and behavioral but not morphologic transition (Josson, Cancer Res 2011). ARCaP_{E} cells, exposed to 200 ng/ml recombinant RANKL protein, gradually changed their morphology from cobble-stoned clusters to more dispersed and fibroblastic-like appearance over the 8-day period (FIG. 7A). RANKL treatment down-regulated E-cadherin but up-regulated N-cadherin expression, reflecting a ‘cadherin switch’ with a replacement of E-cadherin by N-cadherin during EMT (FIG. 7B-1). RANKL also induced up-regulation of Snail, vimentin, and RANKL expression, consistent with the previous observation that these markers are associated with EMT. The ability of recombinant RANKL to induce its own expression is restricted to cancer such as prostate (ARCaP and LNCaP) and osteosarcoma (MG-63 and Saos-2) cells but not normal prostate (PrEC and RWPE-1) or bone (RAW264.7) cells. OPG, a decoy receptor of RANKL, was found to antagonize RANKL-RANK interaction blocked RANKL-induced EMT and cadherin switch in ARCaP_{E} cells (FIG. 7B2). Further, FIG. 7B-3 shows by exposing ARCaP_{M} to OPG, EMT was reversed, both morphologically (reversal to epithelial morphology) and biochemically (expression of mesenchymal cell associated genes and reversal of cadherin switch). These results were confirmed by a genetic approach where depleting endogenous RANKL by RANKL siRNA induced mesenchymal to epithelial transition (MET) (FIG. 7B-4). The functional role of exogenously added RANKL in the induction of EMT was also confirmed in LNCaP cell model (FIG. 7B-5).

The functional role of RANKL in provoking EMT in ARCaP cell model is also supported by the in vitro migration and invasion behaviors of ARCaP and LNCaP cells. Recombinant RANKL treatment increased the migration and invasion of ARCaP_{E} and LNCaP cells, and these changes were abrogated by the presence of OPG (FIG. 7C). Likewise, ARCaP_{M} and LNCaP-RANKL cells expressed higher intrinsic levels of RANKL and also gained increased migratory and invasive phenotypes, which were antagonized by OPG treatment. These results are consistent with the presence of an autocrine loop involving RANKL-RANK that is capable of driving morphologic, biochemical and/or behavioral transition of ARCaP and LNCaP cells from their lesser invasive epithelial phenotype to a more aggressive mesenchymal phenotype.

### Example 38

### Ectopic RANKL Expression Promotes EMT in Human Prostate Cancer Cells

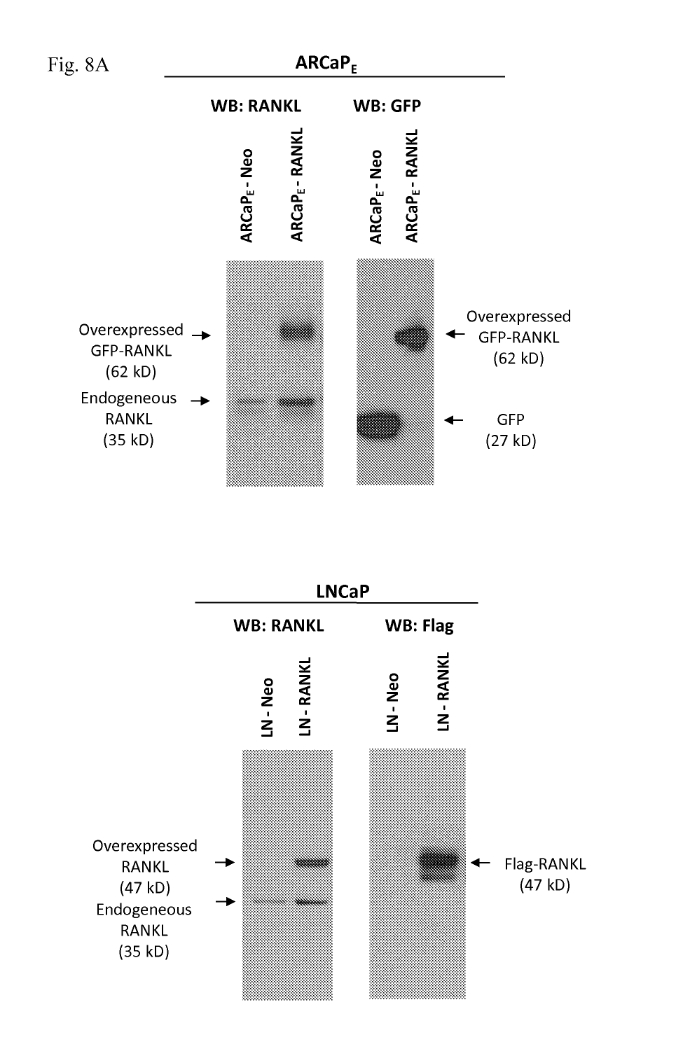

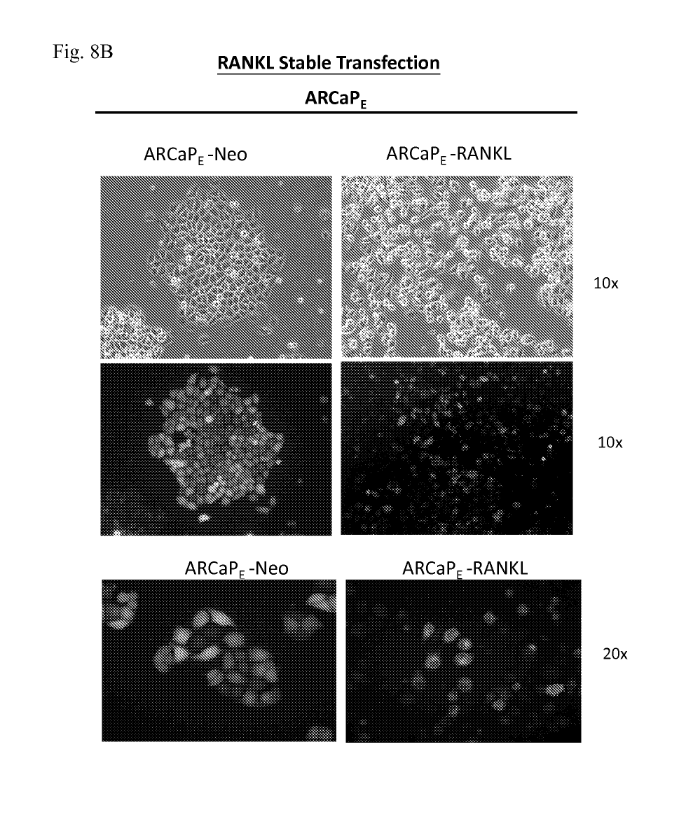

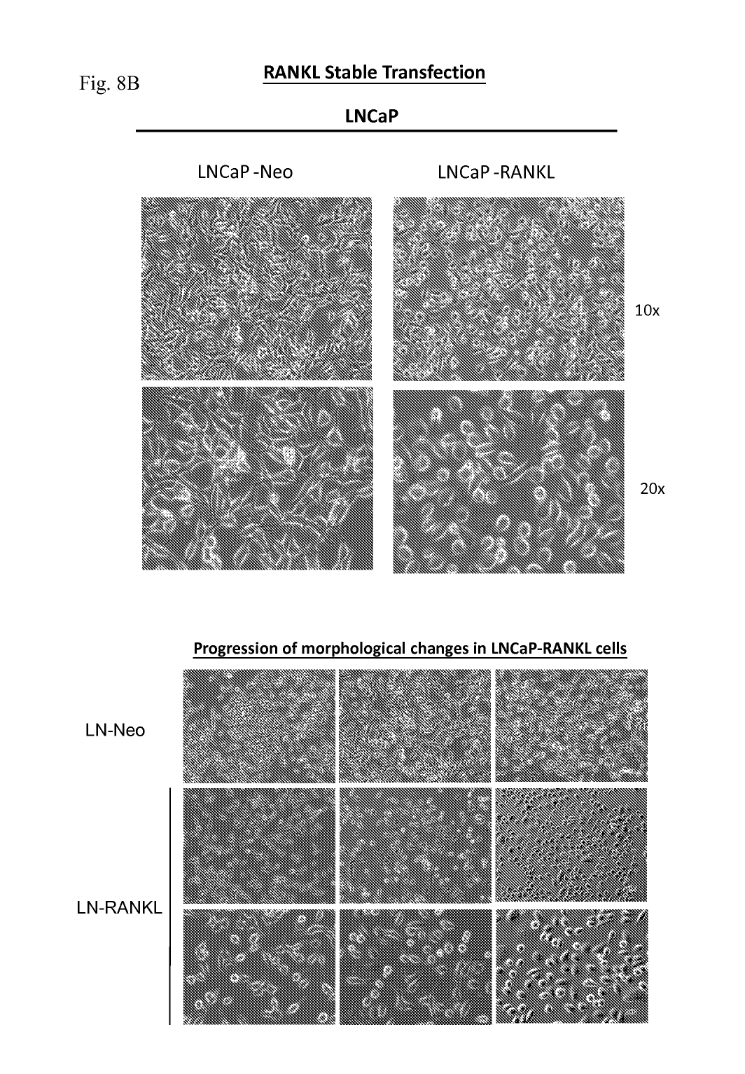

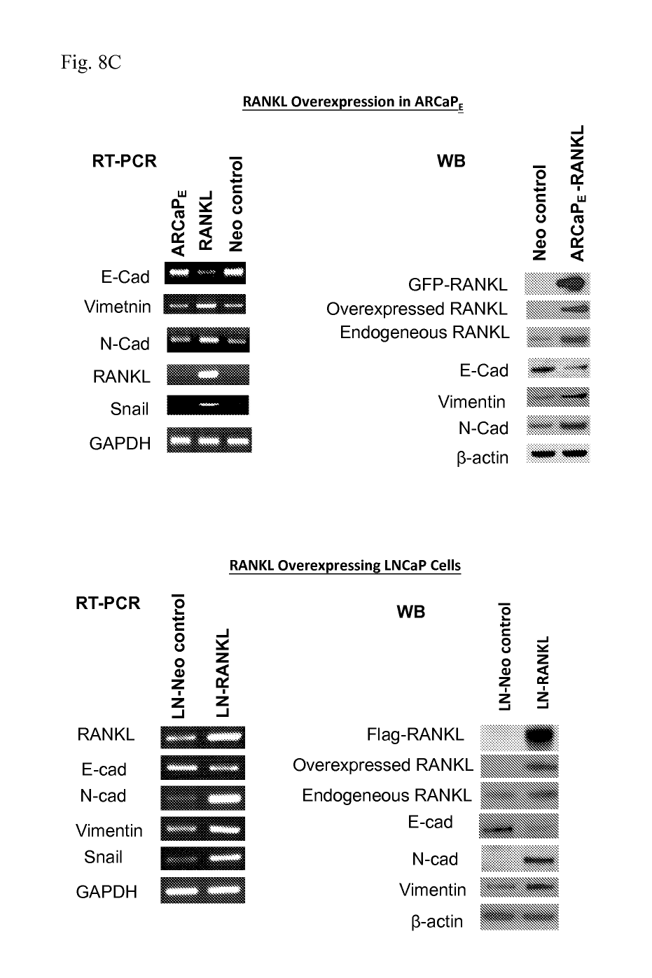

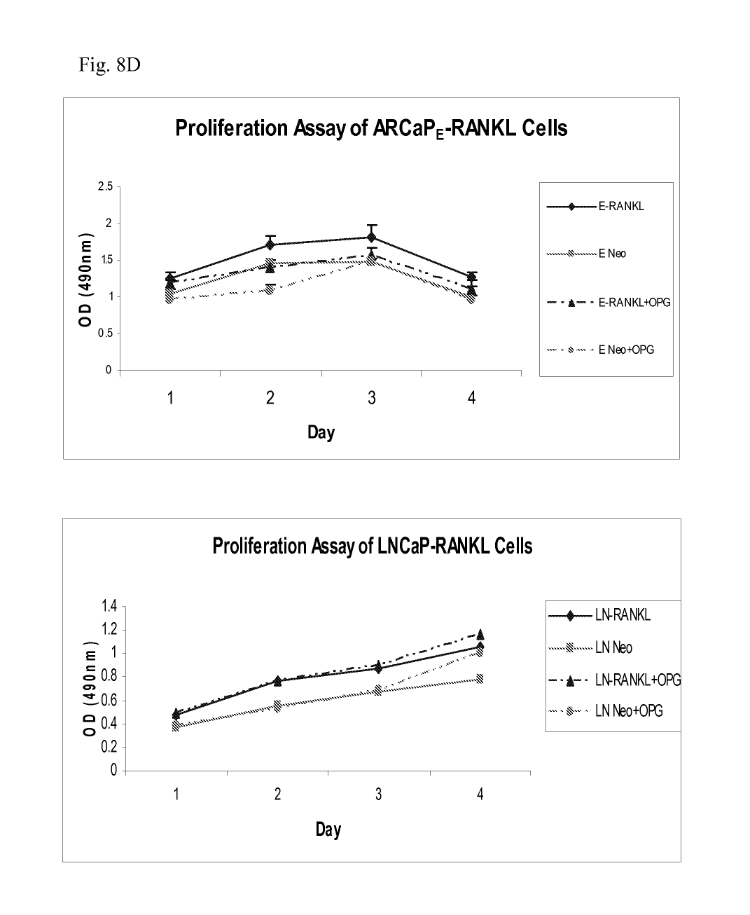

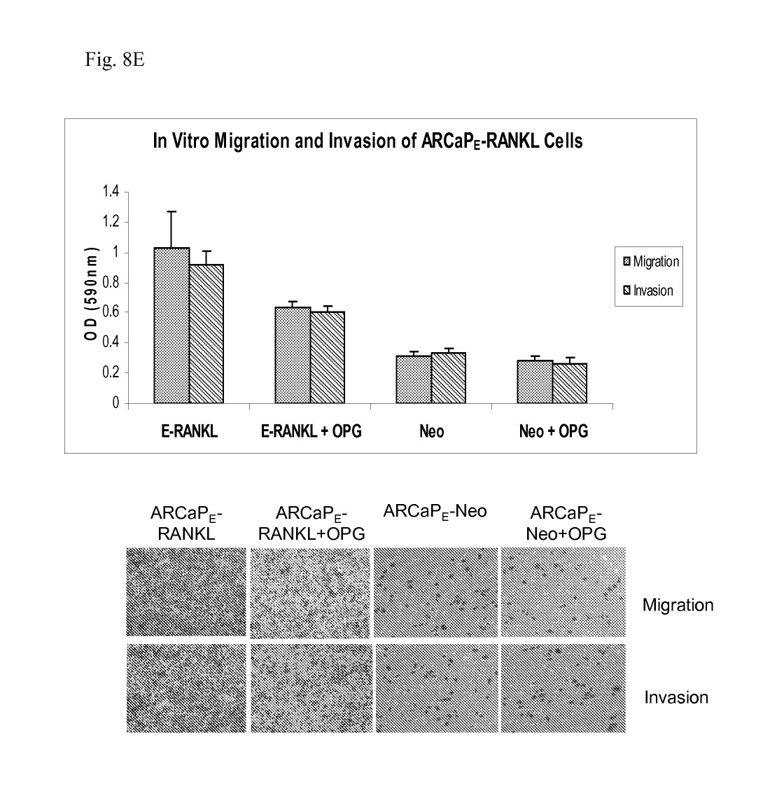

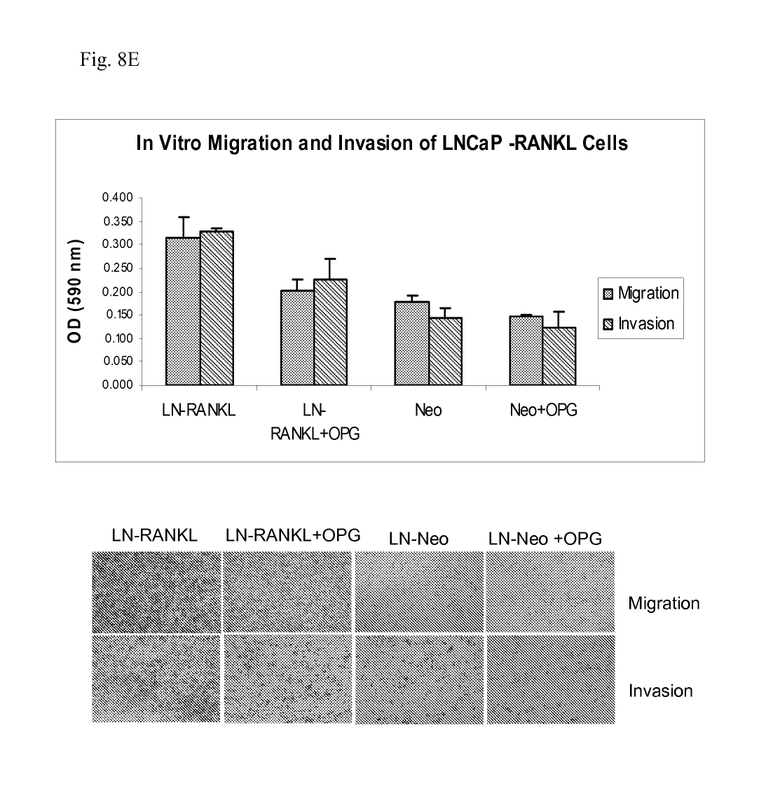

To establish a genetic basis defining the autocrine roles of RANKL in the promotion of EMT and prostate cancer progression, ectopically RANKL, tagged with either GFP or Flag, was enforced respectively in ARCaP_{E} and LNCaP human prostate cancer cell lines. RANKL stably expressing clones were isolated, characterized and in vivo tumorigenic and metastatic potential determined. Western blot analysis demonstrated the expression of RANKL with slightly shifted molecular weight corresponding to the sizes of GFP or Flag tag (FIG. 8A). ARCaP_{E}-RANKL showed morphologic transition to a mesenchymal phenotype, from cobblestone to more dispersed spindle-shaped appearance, when compared to the neo transfected controls (FIG. 8B). A lesser morphologic change was seen in LNCaP-RANKL cells, from their original spindle- and neuron-like morphology to a more round and oval shape morphology, when compared to neo-transfected LNCaP cells. These morphologic features induced by increased RANKL expression were also reflected in their biochemical properties, i.e. a switch of E-cadherin to N-cadherin, vimentin, Snail, and RANKL expression. Moreover, RANKL overexpression in both ARCaP_{E} and LNCaP cells also stimulated endogenous RANKL expression at both transcript and protein levels (FIG. 8A), supported earlier results where adding exogenous recombinant RANKL to these human prostate cancer cells induced RANKL expression (see FIG. 8B). The functional roles of RANKL on ARCaP_{E} and LNCaP cells were determined in vitro. Enforced RANKL exerted no growth effects on either ARCaP_{E} and LNCaP cells (FIG. 8D). ARCaP_{E}-RANKL and LNCaP-RANKL transfectants, however, displayed enhanced migration and invasion compared to that of the Neo control cells (FIG. 8E). The increase in cell migration and invasion are suppressed by OPG, suggesting increased autocrine RANKL-RANK interaction upon enforced RANKL expression. Moreover, ARCaP_{E}-RANKL and LNCaP-RANKL also promoted in vitro soft agar colony formations, implicating the enhanced tumorigenic potentials of these cells conferred by RANKL expression. Such enhanced tumorigenicity of ARCaP_{E}-RANKL and LNCaP-RANKL cells can be attenuated upon addition of OPG and lowered the number of colony formations in the soft agar (FIG. 8).

### Example 39

### RANKL Expression in ARCaP_{E} and LNCaP Cells Induces In Vitro Osteoclastogenesis

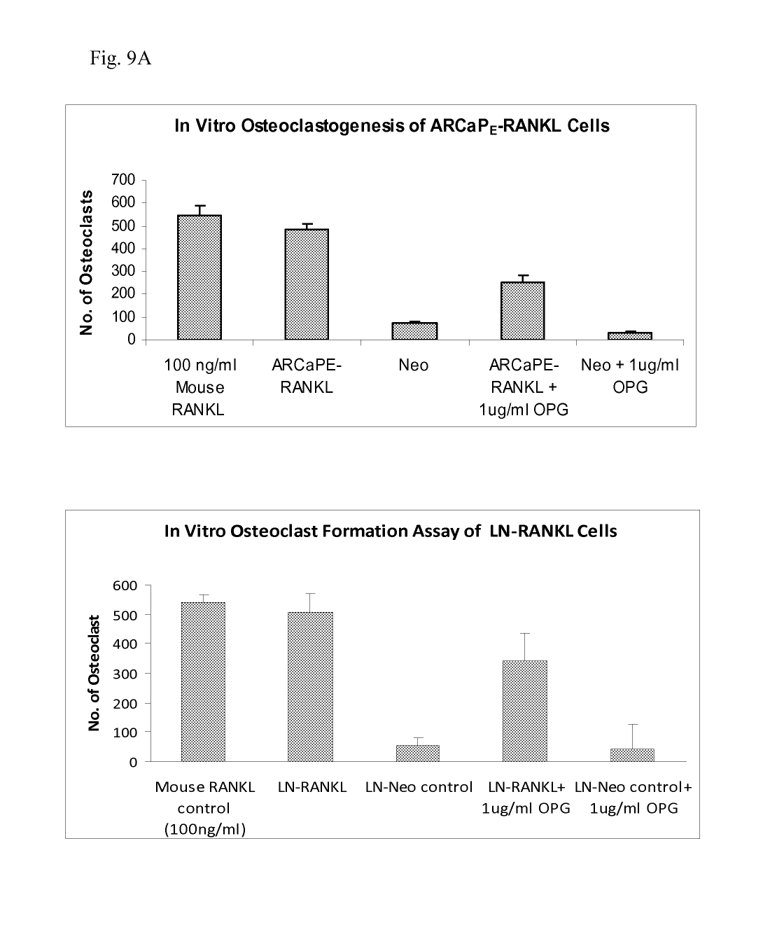

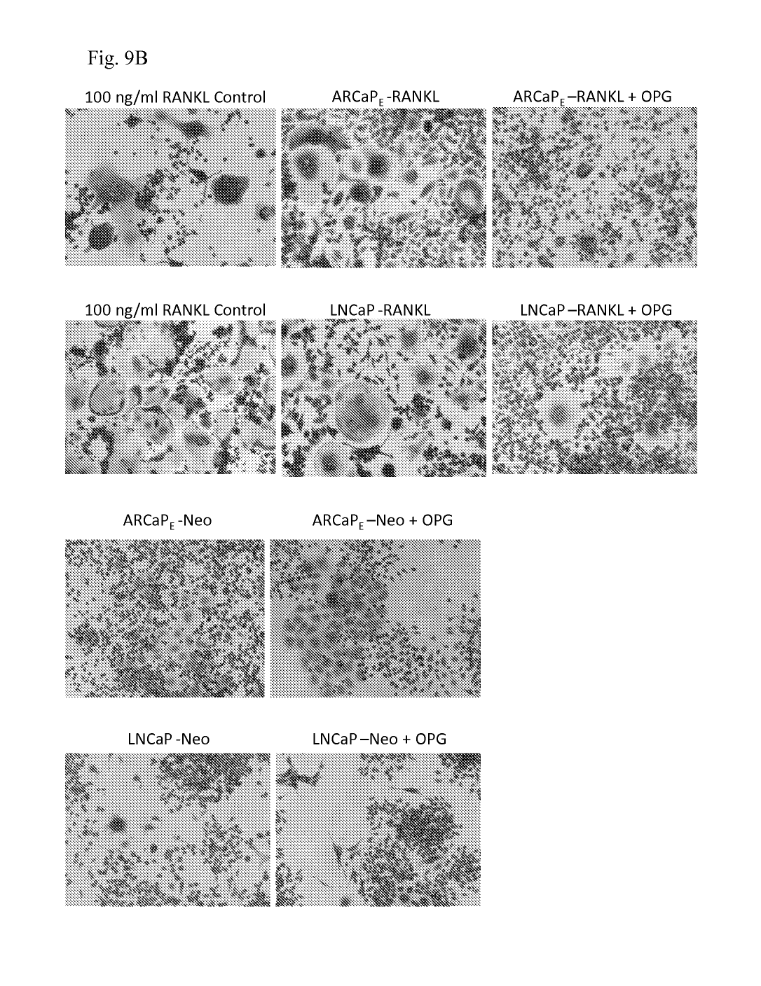

To determine if the enforced RANKL is biologically functional, the inventors compared the ability of both the Neo controls and RANKL-expressed ARCaP_{E} and LNCaP cells with respect to their ability to induce osteoclast maturation in an in vitro osteoclastogenesis assay to quantify the numbers of matured osteoclasts. Mouse macrophage RAW264.7 cells, known to be osteoclast precursors, were co-cultured with ARCaP_{E}-RANKL or LNCaP-RANKL cells; stably Neo-expressed ARCaP_{E} and LNCaP cells serve as controls. Ectopically expressed RANKL in ARCaP_{E} and LNCaP cells induced significantly increased numbers of large and matured osteoclasts (no. of nuclei >3), which were equivalent to that induced by 100 ng/ml of trimerized RANKL protein; ectopically Neo-expressed ARCaP_{E} and LNCaP cells induced only a few mature osteoclasts (FIG. 9). Addition of OPG decreased by approximately two-fold formation of mature osteoclasts, induced by ARCaP_{E}-RANKL and LNCaP-RANKL cells, but had very minimal blocking effects against osteoclastogenesis induced by Neo-transfected control cells. These results further confirmed that RANKL produced by ARCaP_{E}-RANKL or LNCaP-RANKL cells is biologically functional and capable of mounting an enhanced osteoclastogenesis reaction in vitro.

### Example 40

### RANKL Facilitates Tumor Formation and Bone Metastasis of Human Prostate Cancer Cells

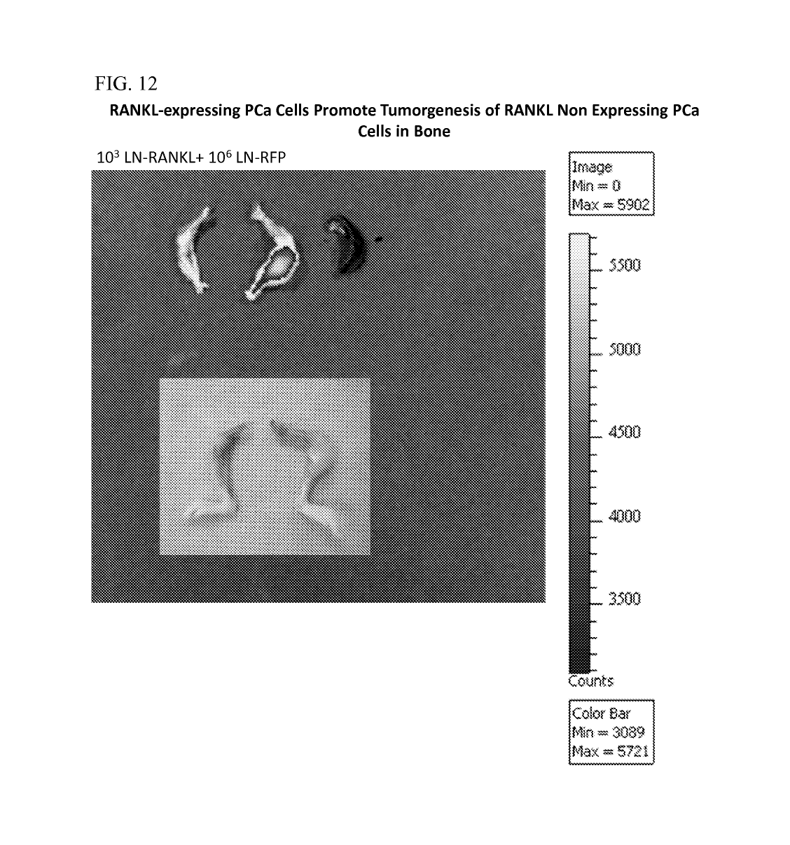

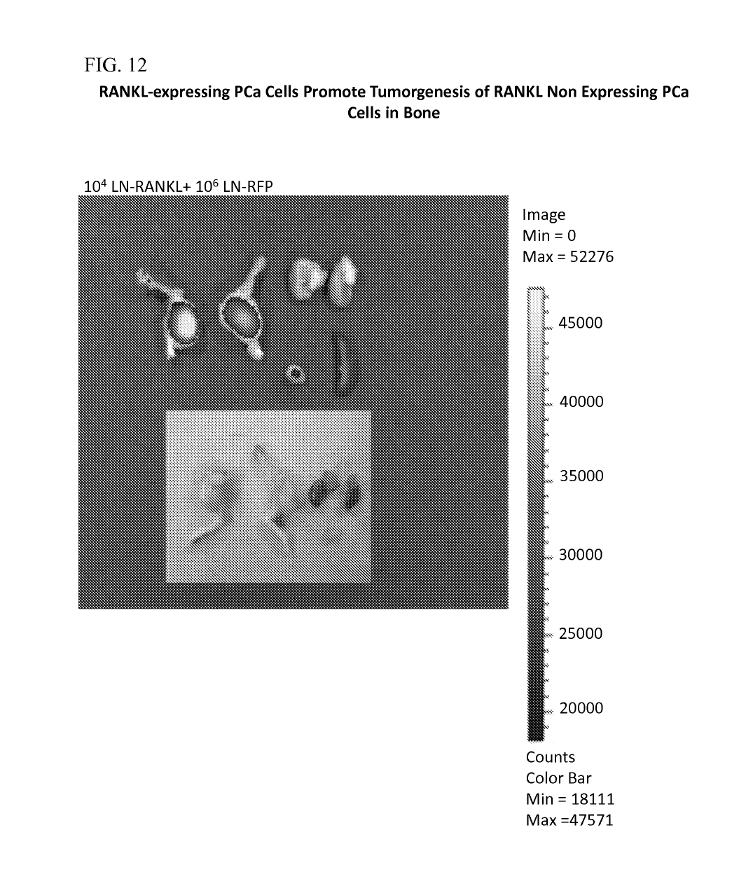

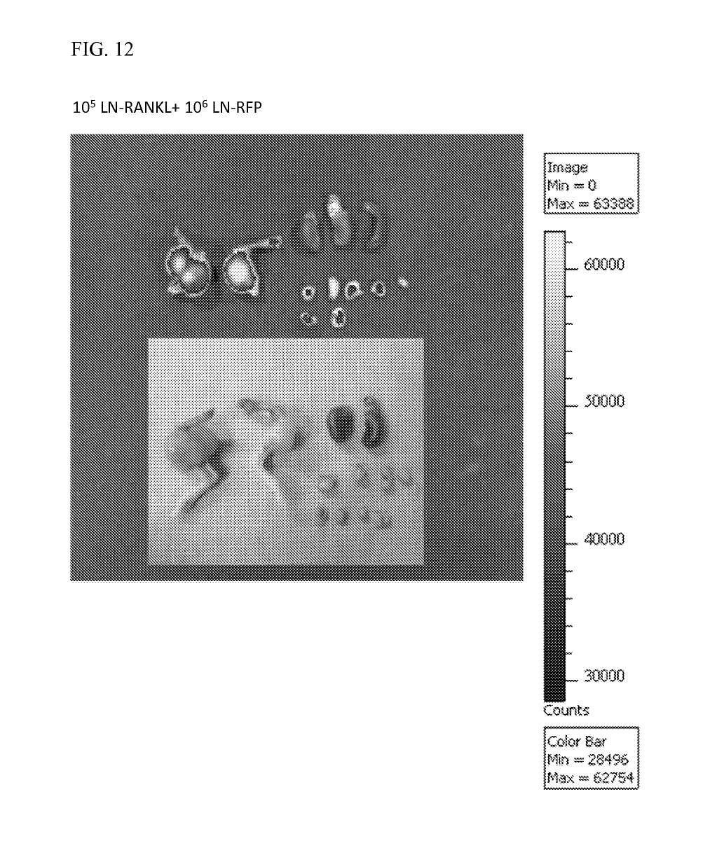

To determine the in vivo effect of RANKL in prostate cancer growth and metastases, the inventors inoculated LNCaP-RANKL or LNCaP-Neo and ARCaP_{E}-RANKL or ARCaP_{E}-Neo cells intracardially into male athymic nude mice to determine their tumorigenic and metastatic potentials. The inventors found that RANKL expression significantly enhanced the metastatic potential of LNCaP and ARCaP_{E} cells. LNCaP-RANKL and ARCaP_{E}-RANKL cells induced 100% and 70% incidence of gross bone metastases to various anatomical sites, including the legs, skull, ribs and spine, and an average of 72% and 65% incidence of gross soft tissue metastases to lymph nodes, adrenal glands, and lungs over a three-month observation period, respectively (Table 3). LNCaP-Neo cells, in contrast, failed to produce any tumor or metastasis, consistent with the known indolent nature of LNCaP cells. ARCaP_{E}-Neo cells also failed to induce any bone metastasis but 20% metastatic incidence to adrenal glands. Mice bearing LNCaP-RANKL and ARCaP_{E}-RANKL cells also developed serious cachexia with extensive tumor burden mostly in the skeleton. The inventors inspected mice with bone lesions using micro-CT (μCT) X-ray tomography to determine the type of the bone lesions induced by LNCaP-RANKL and ARCaP_{E}-RANKL cells. Previous studies by the inventors' laboratory and others showed that intratibial or orthotopic injection of LNCaP lineage-derived C4-2, and C4-2 B cells into the immune-compromised mice produced mixed osteolytic and osteoblastic bone lesions; LNCaP or LNCaP-Neo cells, when injected intrafemorally or orthotopically in immune-compromised mice, however, failed to form tumors (Table 3). FIG. 11 shows 3D μCT scans of skeletons of mice bearing the LNCaP-Neo, LNCaP-RANKL, ARCaP_{E}-Neo, and ARCaP_{E}-RANKL cells. Extensive bone destruction at femoral, pericranial, jaw, and spinal sites was observed in most of the mice bearing the LNCaP-RANKL and ARCaP_{E}-RANKL cells with gross skeletal metastases but not in mice bearing the Neo control cells. Because LNCaP-RANKL cells provoked osteoclastogenesis in mouse skeleton, the inventors tested the hypothesis if these characteristics of LNCaP-RANKL cells may facilitate the growth and colonization of an otherwise non-tumorigenic, non-metastatic and RANKL-null LNCaP cells. The inventors co-inoculated intratibially one million mixtures of LNCaP cells stably tagged with RFP plus an increasing number of LNCaP-RANKL cells, from 1,000, 10,000 to 100,000. The inventors noted tumor formation at both tibial and soft tissues sites with the latter metastases originated from mouse tibia. Tibial bones and soft tissue tumors were harvested and found to contain LNCaP-RFP cells (FIG. 12). As low as one thousand LNCaP-RANKL cells are sufficient to facilitate the growth of LNCaP-RFP tumors in mouse skeleton and soft tissues. Bigger tibial tumors with more intensified RFP signals were detected in the chimeric tumors consisting of an increasing number of LN-RANKL cells.

TABLE 3
Metastatic Incidence of LNCaP-RANKL and ARCaPE-RANKL
Cells in Nude Mice

| | | Bone | | |
|---|---|---|---|---|
| | | Metastases | | |
| Metastatic Sites | | (limbs, rib, | | |
| Cells | Lymph node | jaw, skull) | Adrenal gland | Lung |
| LNCaP Neo | 0/15 | 0/15 | 0/15 | 0/15 |
| Control | | | | |
| LNCaP-RANKL | 18/20 | 20/20 | 17/20 | 8/20 |
| | (90%) | (100%) | (85%) | (40%) |
| ARCaP_{E}-Neo | 0/5 | 0/5 | 1/5 | 0/5 |
| Control | (0%) | (0%) | (20%) | (0%) |
| ARCaP_{E}-RANKL | 6/10 | 7/10 | 7/10 | 0/10 |
| | (60%) | (70%) | (70%) | (0%) |

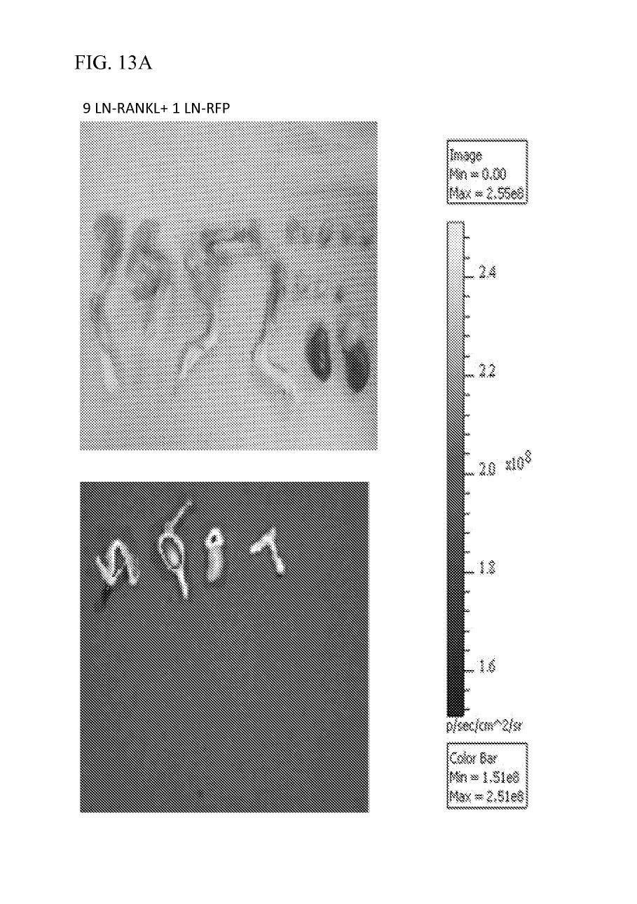

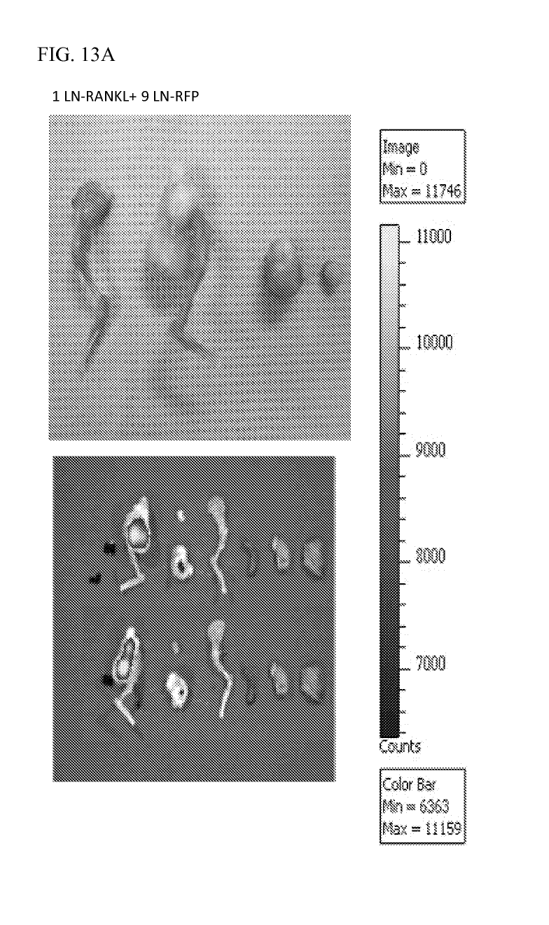

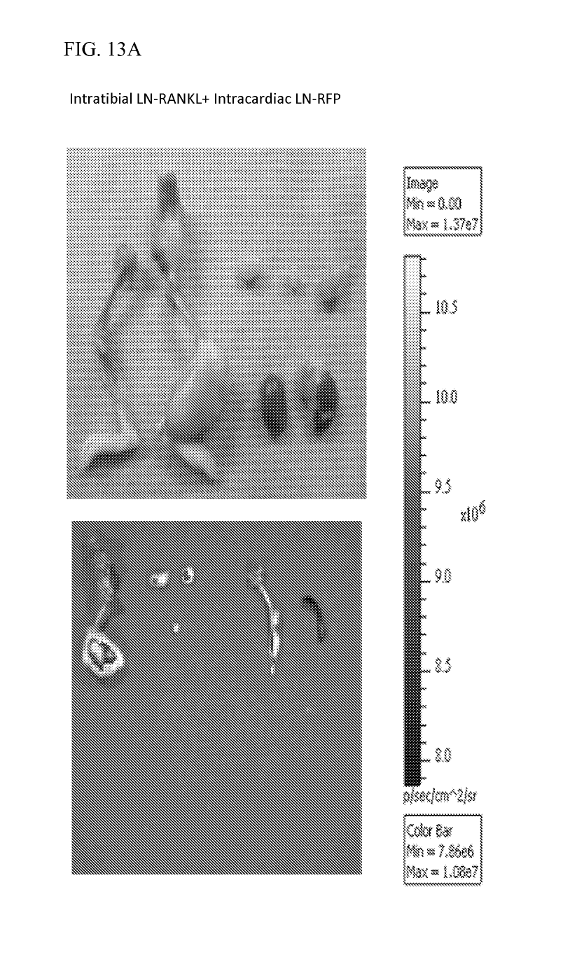

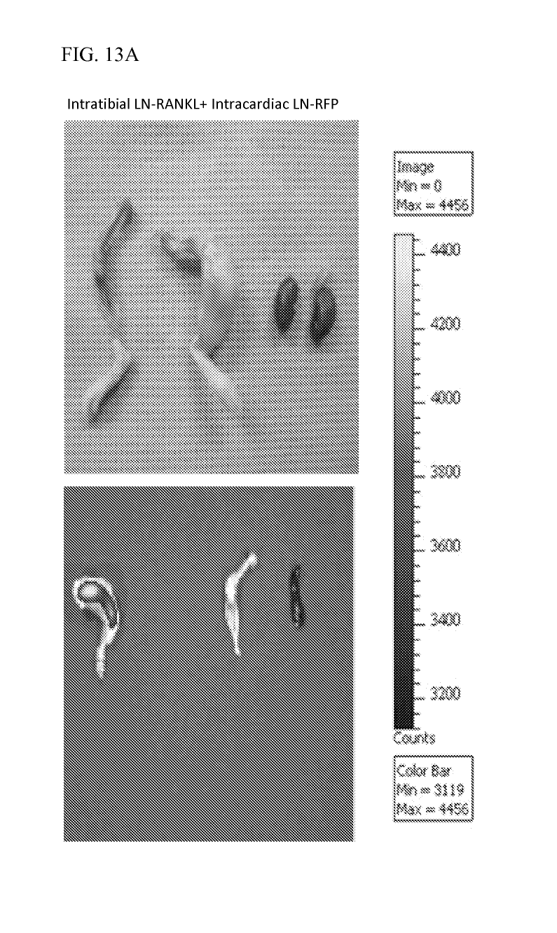

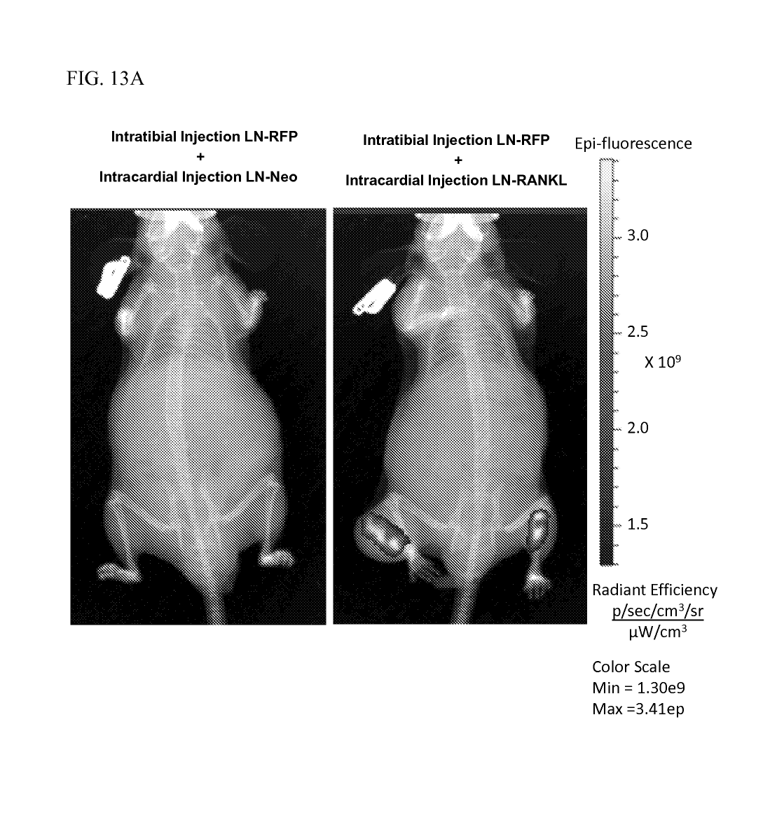

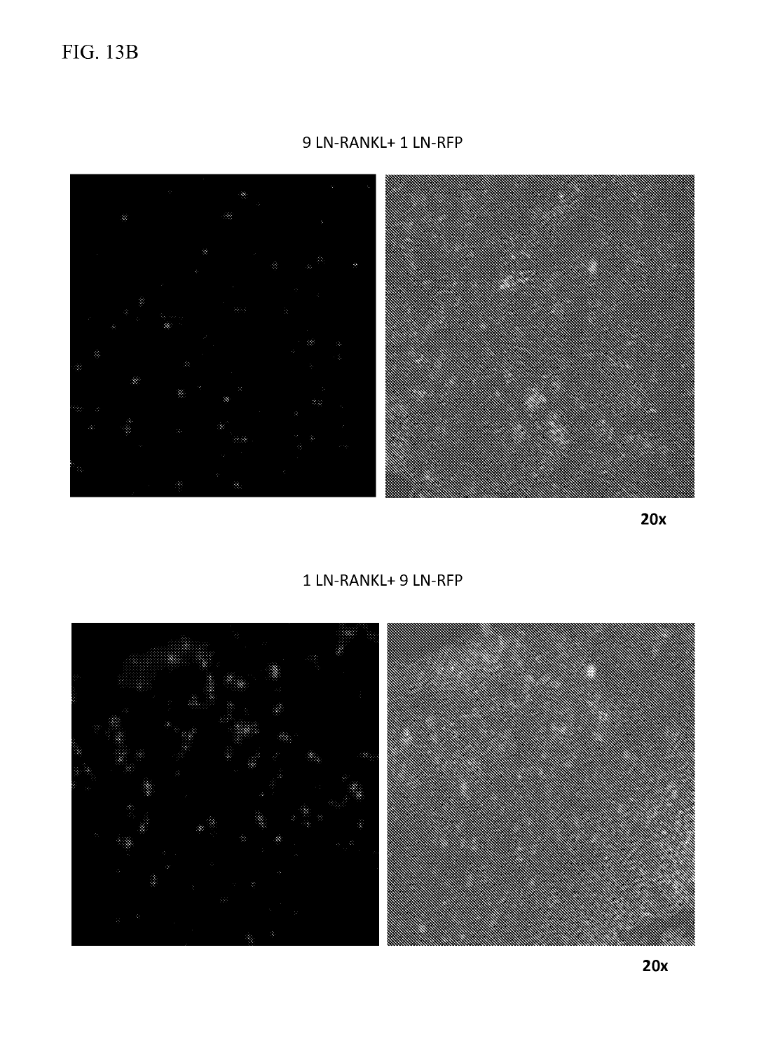

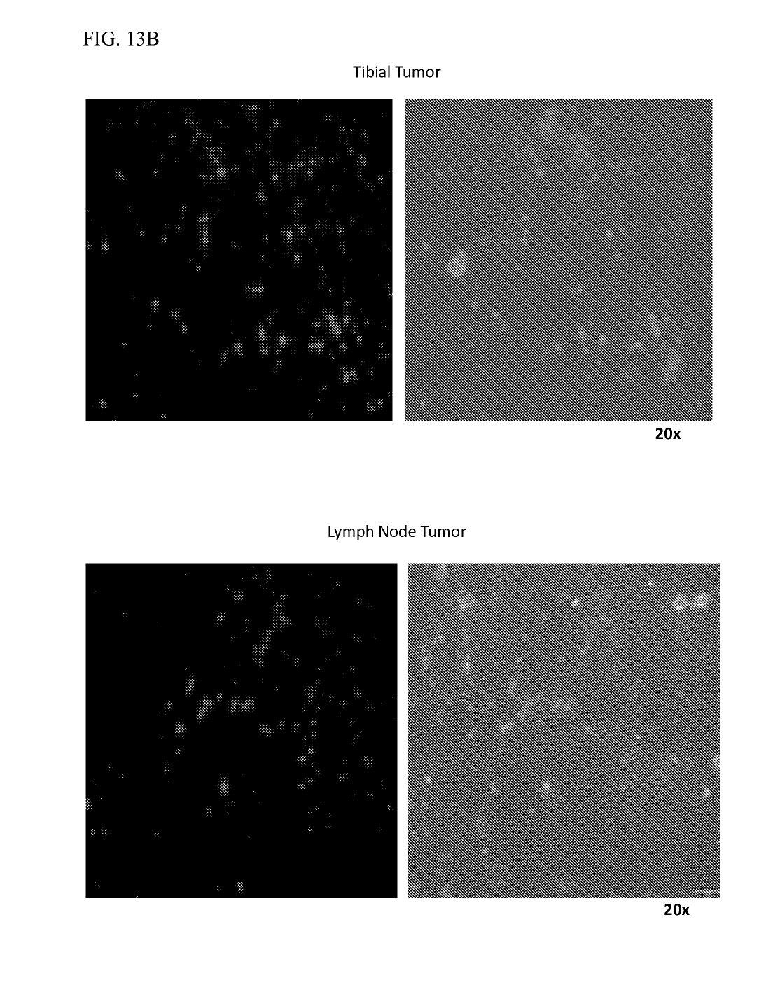

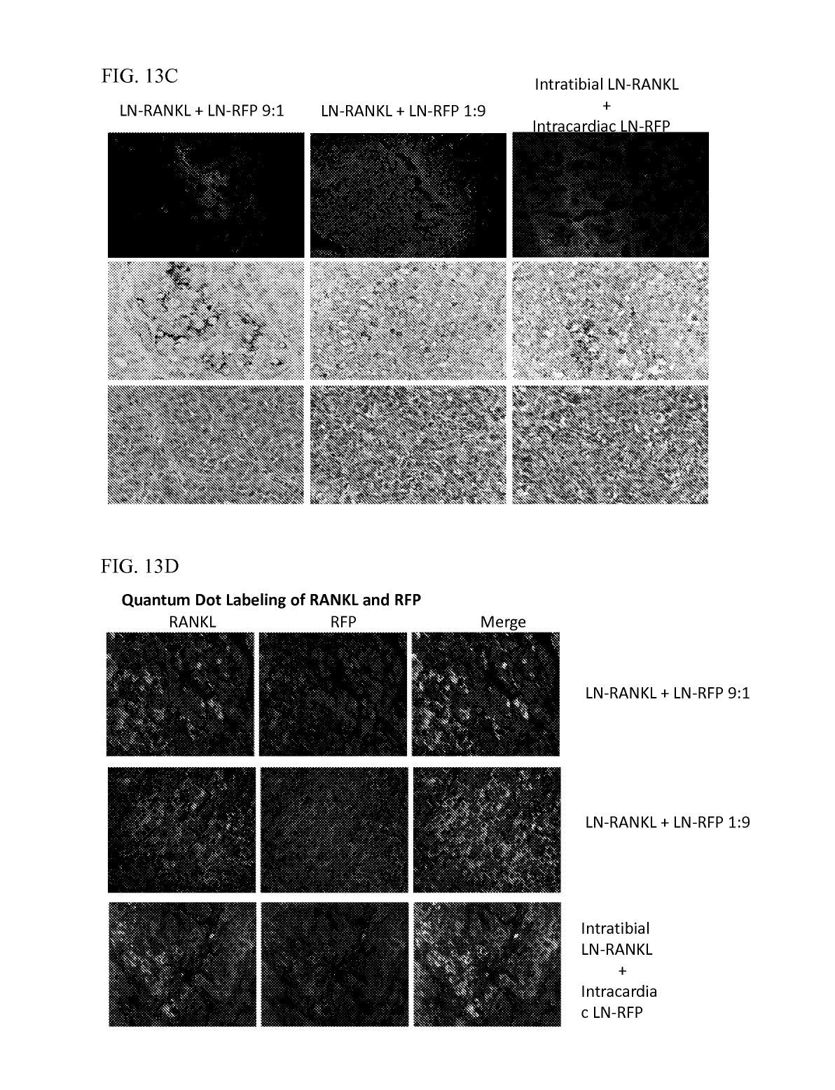

Similar observations were made when one million of mixed LNCaP-RANKL and LNCaP-RFP cells were coinoculated intracardially at a ratio of 1:9 or 9:1, or when one million of LNCaP-RFP cells were injected intracardiacally in mice harbored with one million of LNCaP-RANKL cells in mouse skeleton. These experiments demonstrated unequivocally the ability of non-tumorigenic or “dormant” LNCaP-RFP cells to participate tumorigenesis and metastases to soft tissues in the presence of LNCaP-RANKL cells (Table 4). Representative images and corresponding red fluorescent signal intensity of bone and soft tissue tumors harvested from nude mice bearing tumors of mixed LN-RANKL plus LN-RFP cells are shown in FIG. 13A.

Table 4 demonstrates the metastatic incidence of LN-RANKL plus LN-RFP cells in nude mice. LN-RANKL and LN-RFP cells were either intracardially co-inoculated into male athymic nude mice at a ratio of either 1 to 9 or 9 to 1 in a total of a million cells. Additionally, one million of LN-RANKL cells were also inoculated intratibially followed by intracardiac implantation of a million of LN-RFP cells. Results of these studies showed that mice bearing the LN-RANKL plus LN-RFP at 9 to 1 ratio demonstrated 100% metastases to bone and an average of 65% metastases to the soft tissues, such as adrenal glands and lymph nodes; when co-inoculation of LNCaP-RANKL and LNCaP-RFP cells with a ratio of 1 to 9, however, the metastases to bone and soft tissues reduced to 60% and 30% incidence, respectively. Whereas, intracardial co-inoculation of half a million of LN-Neo and half a million of LN-RFP cells failed to induce any metastasis. Additionally, when inoculating one million LN-RANKL cells intratibially followed by intracardial inoculation of one million LN-RFP cells, all of the mice (n=5) demonstrated 100% tumor growth in the tibial bone as well as metastases to the soft tissues of either adrenal glands or lymph nodes with evidence of the participation by both cell types in the chimeric tumors.

TABLE 4
RANKL-expressing PCa Cells Promote Co-colonization of
Non-metastatic and RANKL-non-expressing PCa Cells to
the Metastatic Sites

| | Metastatic Sites | | |
|---|---|---|---|
| | | Adrenal | |
| Groups | Bone | Gland | Lymph Node |
| Intracardial Injection of | 0/10 | 0/10 | 0/10 |
| 5 × 10⁵ LN-Neo + 5 × 10⁵ LN-RFP | | | |
| Intracardial Injection of | 10/10 | 8/10 | 5/10 |
| 9 × 10⁵ LN-RANKL + 10⁵ LN-RFP | | | |
| Intracardial Injection of | 6/10 | 0/10 | 3/10 |
| 10⁵ LN-RANKL + 9 × 10⁵ LN-RFP | | | |
| Intratibial Injection of 10⁶ LN-RANKL + | 5/5 | 3/5 | 2/5 |
| Intracardial Injection of 10⁶ LN-RFP | | | |

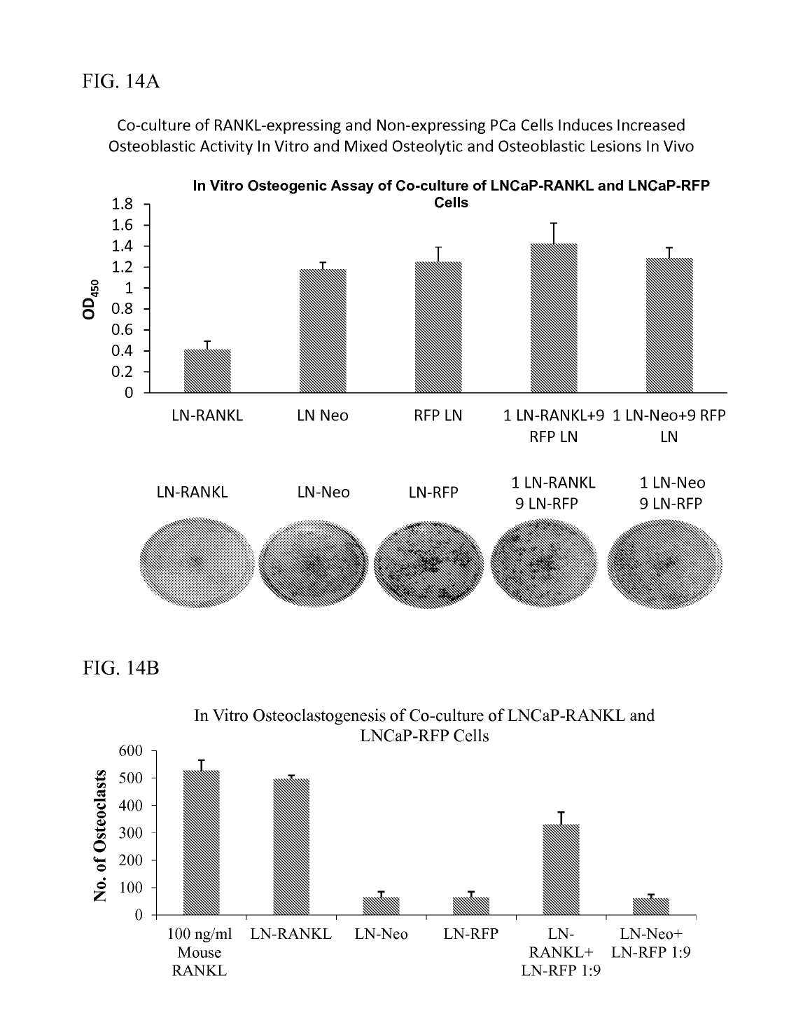

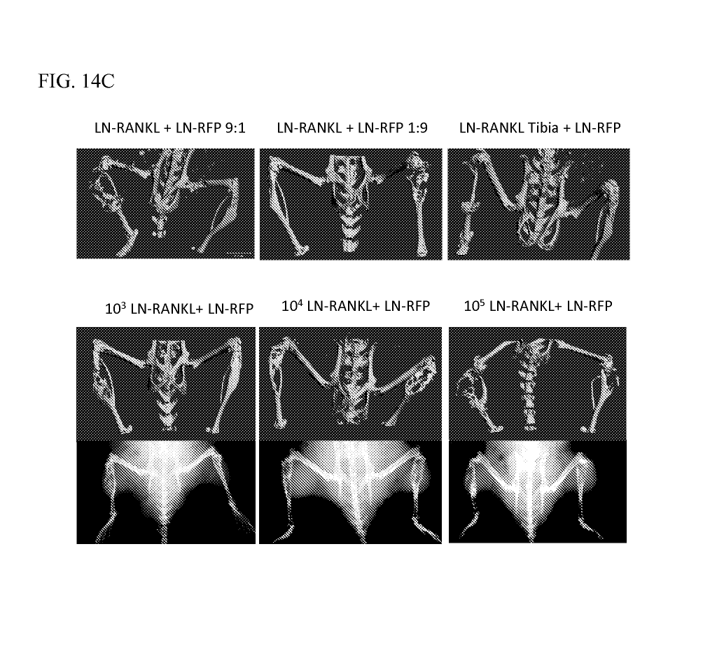

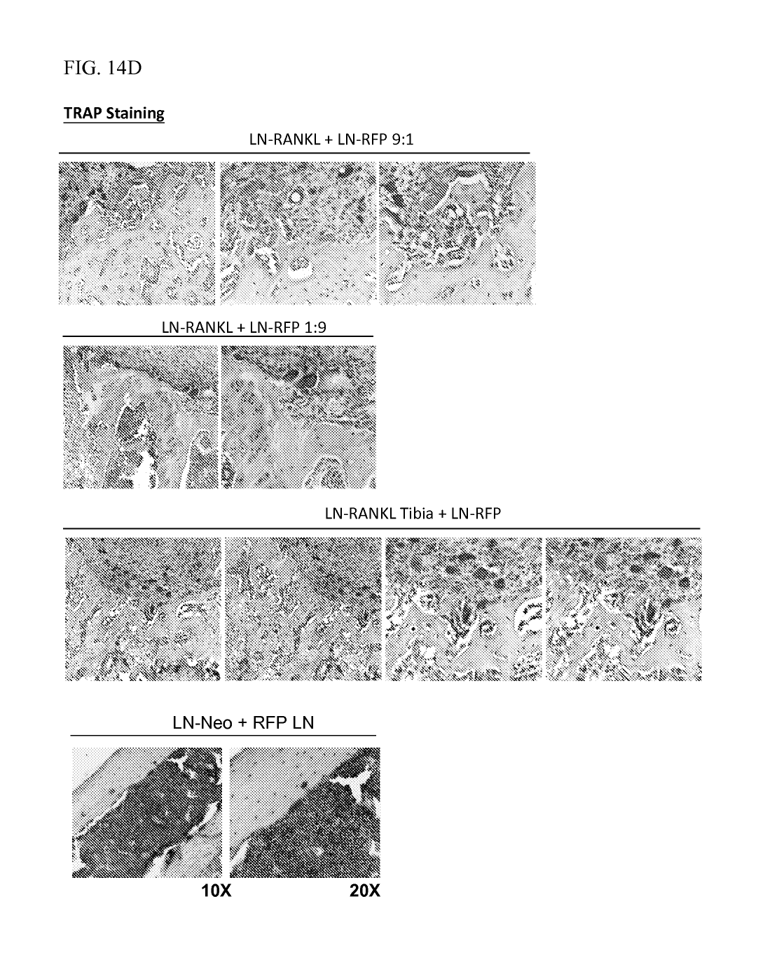

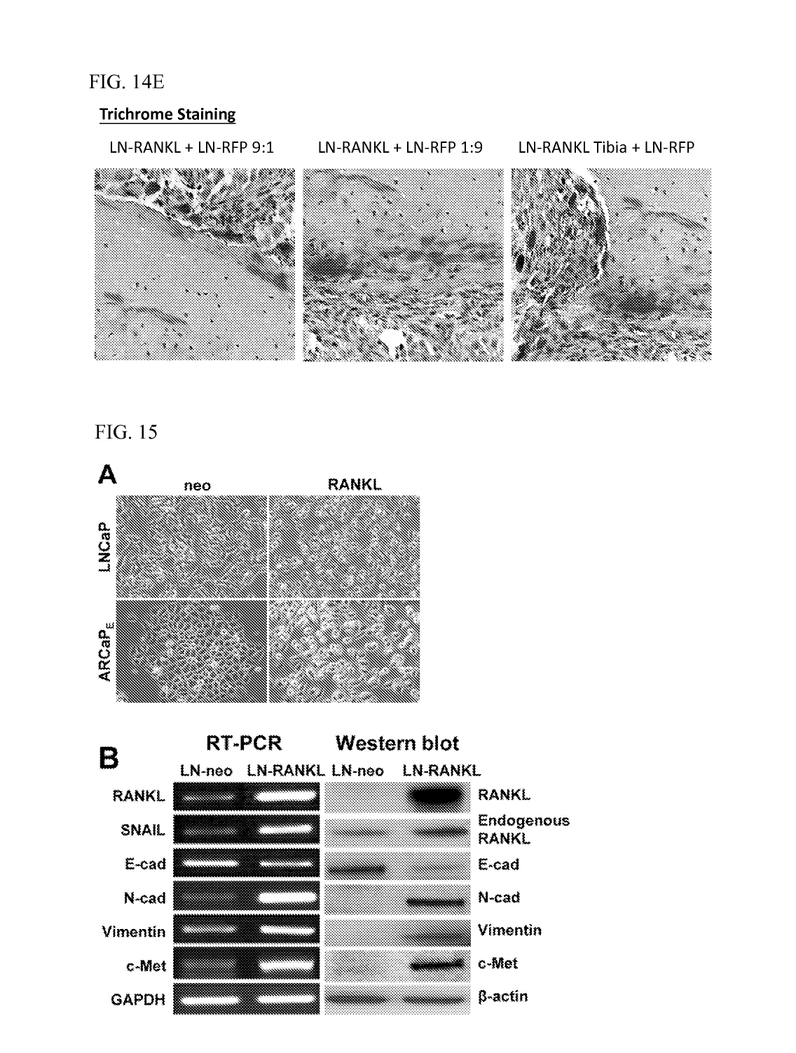

Since LNCaP-RANKL cells induced predominately osteolytic reactions in mouse skeleton, the inventors examined whether a mixed osteolytic/osteoblastic reactions can be provoked by the co-presence of RANKL-expressing and -null LNCaP cells. Osteogenic assay in vitro showed that LN-RANKL cells induced predominately osteoclast differentiation with low level of osteoblast mineralization while LN-Neo or LN-RFP cells either cultured alone or together with LN-RANKL cells produced substantially increased osteoblast mineralization (FIG. 13B). These results are in agreement with the 3D μCT scans of representative chimeric tumors comprised of 1 to 9 or 9 to 1 ratio of LN-RANKL and LN-RFP cells or intratibial LN-RANKL plus intracardial LN-RFP cells all displayed mixed osteolytic and osteoblastic lesions in the bone (FIG. 14). TRAP staining of mature osteoclast lining (red) and orange G staining of new bone formation (yellow orange) in bone tumor were observed in the chimeric tumors co-inoculated with LN-RANKL and LN-RFP cells in nude mice (FIG. 14D). An alternative evidence of the increased level of osteoblastic activity in the tumors induced by the co-inoculation of LN-RANKL and LN-RFP cells is illustrated by detecting the osteoid formation/thickness using Trichrome staining (FIG. 14E). The osteoid stained in red, and the mineralized bone stained in turquoise. The level of osteoid formation and thickness can reflect the level of bone formation. Together, these results indicated that the level of osteoblastic and osteoclastic phenotype displayed in the bone lesions seemed to be correlated with the number of LN-RFP and LN-RANKL cells inoculated, respectively during the co-administration protocol. Together, the inventors observed that the extent of osteolysis can be switched from osteolytic to osteoblastic dependent upon the proportion of RANKL-expressing and RANKL-null prostate cancer cells used in the mixed populations.

The chimeric tumor specimens were subjected to histochemical analyses of RANKL expression and EMT phenotype using IHC staining against flag-tagged RANKL and EMT markers. The inventors observed that RANKL expression, when detected with either anti-Flag or -RANKL antibody showed stronger but heterogenous signals in prostate tumors in the bone and at bone/tumor interface compared to the prostate tumors in the lymph node. Both bone and lymph node metastatic prostate tumors expressed high levels of N-cadherin and vimentin but low level of E-cadherin, demonstrating the underlying EMT phenotype upon progression to metastases (FIG. 11C). Interestingly, there were also clusters of E-cadherin positive tumor cells at both bone and lymph node metastatic sites, suggesting a reversal of EMT or mesenchymal-to-epithelial transition (MET) took place, when tumor cells resided at metastatic sites (FIG. 11C). Since RANKL expressed by LNCaP cells was found to be functional in promoting osteoclastogenesis, the inventors examined LNCaP-RANKL-induced osteoclastgenic activity in the mouse skeleton by TRAP staining. The inventors observed an increased number of TRAP-positive mature osteoclasts at the bone/tumor interface compared to the normal bone from the mice bearing the LNCaP-Neo cells (FIG. 11D), further confirms the roles of RANKL, secreted by prostate cancer cells, which accounts for increased host osteoclast maturation and subsequent bone resorption and destruction at the skeleton where prostate cancer colonizes.

### Example 41

### RANKL Promotes EMT by Activation of NF-κB Through an Akt and P38 Dependent Mechanism in Prostate Cancer Cells

The autocrine action of tumor cell-derived RANKL on the cell surface receptor RANK in prostate cancer cells was studied. RANKL expressed in both ARCaP_{E} and LNCaP cells significantly activated PI3K-Akt, P38 MAPK, and NF-κB signaling pathways by increasing the phosphorylated levels of Akt on serine 473, P38 on tyrosine 180, and P65 on serine 536; nevertheless, RANKL did not activate other MAPKs, such as Erk1/2 and JNK/SAPK (FIG. 10A). RANKL-RANK interaction is therefore capable of inducing multiple downstream signaling pathways in prostate cancer cells similar to RANK activation in osteoclasts. Subsequently, the inventors examined the effect of kinase inhibitors specific for Akt, P38, and NF-κB on RANKL-induced EMT. The inventors treated both ARCaP_{E}-RANKL and LNCaP-RANKL cells with 20 μM of a PI3K inhibitor, LY294002, 20 μM of a P38 inhibitor, SB203580, and 2 μM of NF-κB or proteasome inhibitor, PS341 respectively for 4 h followed by western blot analysis. As can be seen in FIG. 10B, PI3K and NF-κB inhibitors both increased E-cadherin and decreased vimentin and RANKL expression in ARCaP_{E}-RANKL cells, leading to a reversal of EMT, and similar results were observed in LNCaP-RANKL cells treated with either a P38 or a NF-KB inhibitor. This result shows that direct inhibition of NF-κB signaling prevented RANKL-induced EMT in both ARCaP_{E}-RANKL and LNCaP-RANKL cells. Moreover, the inventors observed that PI3K inhibitor also reduced the phosphorylated level of P38 and subsequently, P38 inhibitor resulted in a reduction in phosphorylated level of P65 in both cell types, thus suggesting transactivation of cell signaling axes in the sequence of Akt, P38, and P65 by RANKL stimulation in prostate cancer cells. These findings are consistent with previous studies showing that PI3K-Akt transactivates P38, which further activates NF-κB transcriptional activity through phosphorylation of P65 upon TNF-α stimulation. Therefore, the inventors conclude that RANKL promoted EMT by activation of NF-κB pathway through an Akt and P38 dependent mechanism in prostate cancer cells.

### Example 42

### RANKL Induced cMet Expression in Prostate Cancer Cells

Interestingly, the inventors further discovered that EMT-promoting RANKL and HGF are capable of inducing cMet expression as well as activating downstream cMet signaling through increased level of phosphorylated cMet in prostate cancer cells. As shown in FIG. 15A, exogeneous RANKL and HGF treatments significantly increased cMet expression as well as phosphorylated level of cMet in both LNCaP and ARCaP_{E} cells, and such induction can be abrogated upon OPG and anti-HGF antibody treatment. Similarly, RANKL overexpressing LNCaP cells also up-regulated cMet expression at both RNA and protein levels (FIG. 15). As demonstrated by single quantum dot labeling (SQDL), LNCaP-RANKL cells expressed high levels of membrane-bound cMet and phosphorylated cMet compared to RANKL-low and cMet-null expressing LNCaP-Neo cells (FIG. 15D). Moreover, the induced c-Met is biologically functional since the p-c-Met expression increased upon the addition of HGF, and this activation of p-c-Met can be antagonized by anti-HGF monoclonal antibody (FIG. 15C). Promoter reporter assay further confirmed that RANKL treatment or overexpression induced up-regulation of cMet transcriptional activity (FIG. 15). Up-regulation of cMet and p-c-Met was also observed in histological sections of bone and soft-tissue tumors induced by LNCaP-RANKL cells in vivo.

### Example 43

### Further Discussion of FIGS. 13-15

FIG. 13: The inventors discovered that the colonized LN-RFP cells in the bone tumors also express high or moderate level of RANKL indicated by the co-localization (yellow) of RANKL (green) and RFP (red) labeling (FIG. 13D), suggesting induction of RANKL in RFP-tagged LN cells by LN cells expressing RANKL, but not expressing RFP. This result further implicates that the RANKL-expressing LN cells can “reawaken” the dormancy of LN-RFP cells, leading to their participation of tumorigenesis possibly through the induction of RANKL expression; one million LN-RFP cells, when injected alone, failed to form tumors in mouse skeleton for a 20 week observation period (data not shown). This observation might be well explained by the ‘seed and soil’ theory where the disseminated LN-RANKL cells initially created a favorable environment by inducing the osteolytic lesion in the bone, leading to the release of growth factors and cytokines that subsequently allowed the circulating and presumably dormant LN-RFP cells to colonize and grow at the bone sites. Interestingly, fluorescent imaging also showed intracardial inoculation of LNCaP-RANKL cells in mice harboring LNCaP-RFP cells in the tibial bone induced the skeletal tumor formation of these non-tumorigenic LNCaP-RFP cells as strong intensity of red fluorescent signal was detected in the tibial tumor (FIG. 13A). These experiments together demonstrated unequivocally the ability of non-tumorigenic or “dormant” LNCaP-RFP cells to participate tumorigenesis and metastases to bone and soft tissues in the presence of LNCaP-RANKL cells.

FIG. 14: These results indicated that the level of osteoblastic and osteoclastic phenotype displayed in the bone lesions seemed to be correlated with the number of LN-RFP and LN-RANKL cells inoculated, respectively during the co-administration protocol. Together, it was observed that the extent of osteolysis can be switched from osteolytic to osteoblastic dependent upon the proportion of RANKL-positive and RANKL-negative prostate cancer cells used in the mixed populations. For example, using LN-RANKL cells as a model, it is shown that a small number of LN-RANKL expressing cells are sufficient to induce bone metastases in the presence of bystander RANKL non- or low-expressing cells. Importantly, histomorphology showed that in the bone metastasis tumors, there reside both RANKL-expressing and low-expressing cells, suggesting the important function of RANKL in contributing to the overall bone metastases irrespective to the basal steady-state level of RANKL expression. Since RANKL low and non-expressing LNCaP cells failed to form tumors in mice without the co-presence of RANKL positive LNCaP cells, the inventors believe that RANKL is an important factor activating the “dormancy” of PCa cells to form tumors in mouse bone. This explains well the clinical observation of cancer dormancy in which PCa cells often found in the bone but failed to colonize the bone. In the presence of RANKL positive LNCaPcells, RANKL low or non-expressing PCa cells participated bone colonization. These results have important clinical implication in which further justify the targeting of RANKL as an effective therapeutic approach for human prostate cancerbone metastasis targeting not only the existing tumors but also preventing the activation of tumor dormancy.

FIG. 15: The observation expands the RANKL/RANK/OPG triad system into RANKL/RANK/OPG/c-Met relationship. The important clinical implications of this observation are: a) RANKL and c-Met can be co-targeted to achieve therapeutic synergy. Targeting RANKL can prevent the forward feedback of RANKL induction by RANKL, hence preventing downstream activation of survival and antiapoptotic signaling mediated by the activation of NF-kB and Mcl-1. Targeting c-Met, therefore, can potentially prevent the cascade of downstream signal activation of RANKL which induced c-Met and its downstream signaling. b) Targeting RANKL and c-Met can prevent the “reawakening” of tumor dormancy so that minimize bone colonization of PCa cells. c) Because activation of c-Met signaling results in downstream activation of Mcl-1, an anti-apoptotic member of the bcl-2 family (Zhang, Mol Cancer, 9, 9), mediated by the activation of Src-kinase and Stat-3 which converge upstream signaling from VEGF-neuropilin-1-induced cell signaling, targeting RANKL could downregulatec-Met-VEGF-mediated Mcl-1 expression hence accelerate cancer apoptosis.

### Example 44

### RANKL Predicts Prostate Cancer Bone Metastasis and Lethal Phenotype of Human Prostate Cancer

The inventors found RANKL expression in primary human prostate cancer predicts human prostate cancer survival in patients. These results supported and validated the animal model described herein.

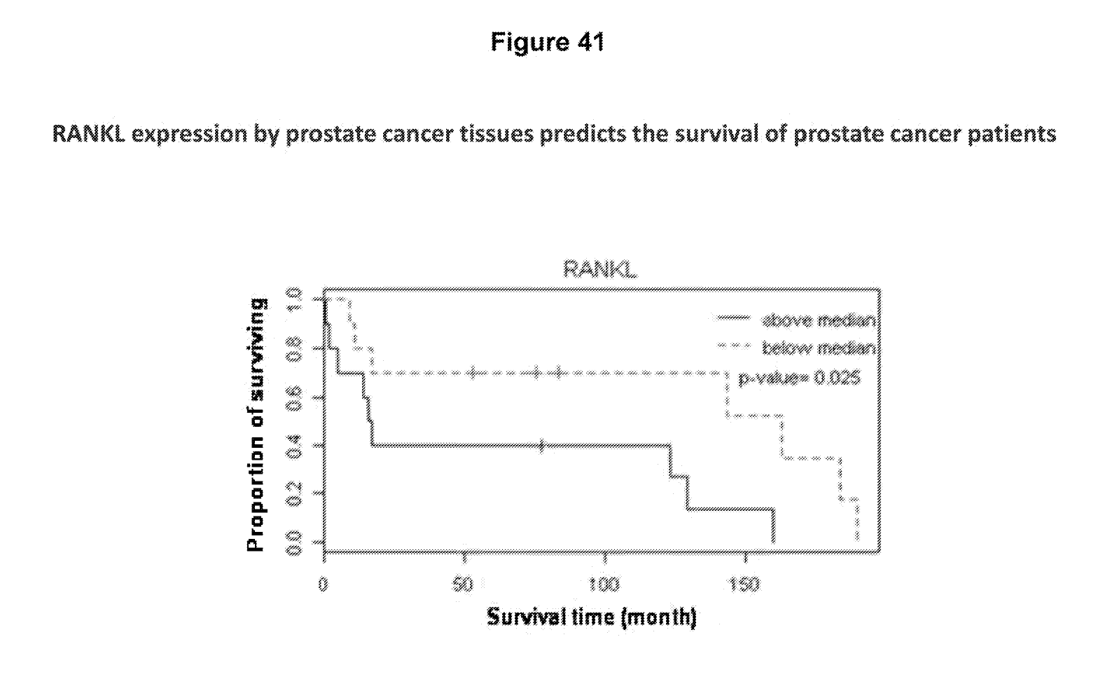

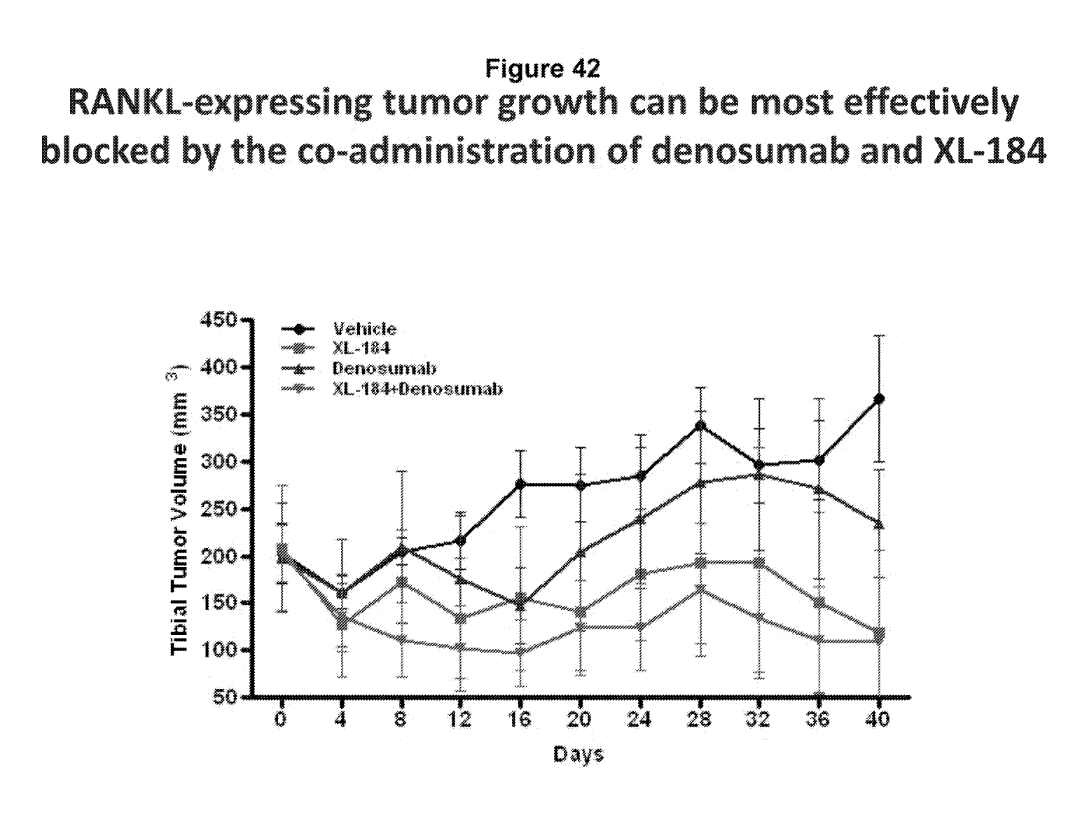

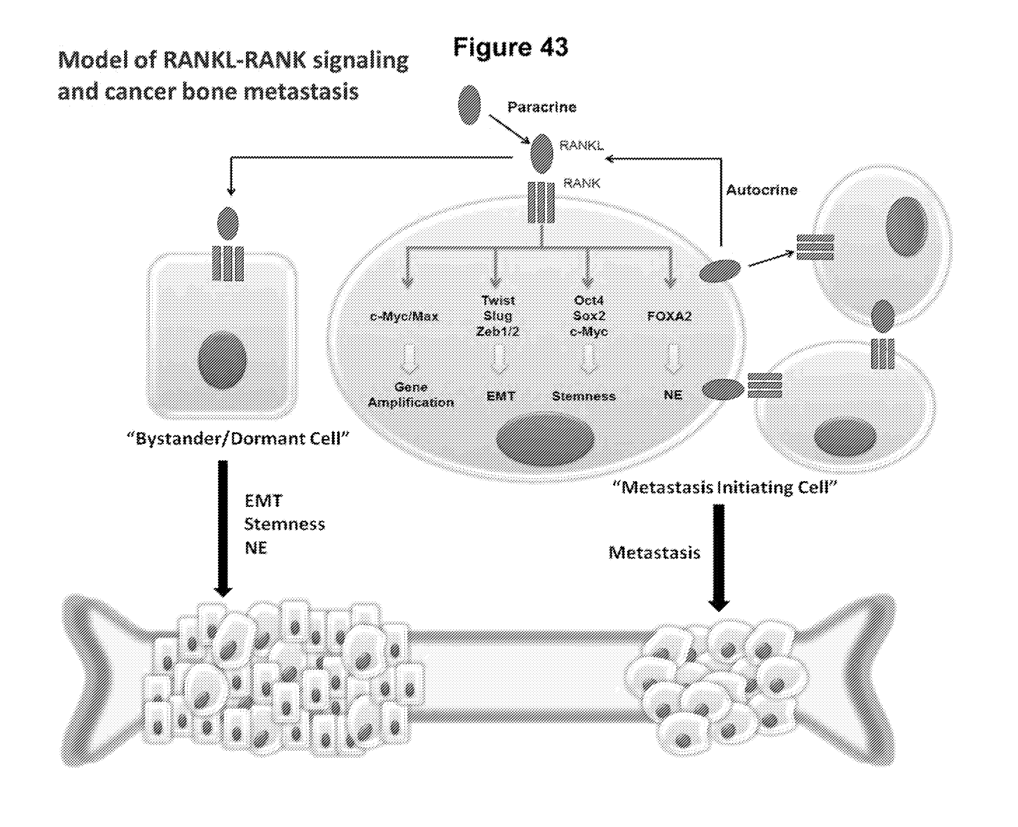

The graph (FIG. 41) shown represent the results obtained from 20 patients with their survival either low or high with about equal distribution. Each of the patients had 2-14 tissue specimens from TURP and are subjected to immunohistochemistry staining with RANKL antibody. RANKL antibody detects RANKL protein expression in these studies. After staining the tissue specimens with anti-RANKL antibody, we evaluated an average of greater than 12,000 single cells and evaluated RANKL distribution in cytosol, cell membrane, and nucleus. By multiplexing quantum dot using an automated Vectra imaging system, the data plotted is RANK order of intensity directly read from the imaging analyzing system. These series of data was then analyzed in a double blind manner. The plot revealed RANKL is a significant biomarker that can differentiate patients with either long (over 100 months) or short survival. In the same assay, the inventors observed that HIF-1a, phosphorylated NF-kB, VEGF, and phosphorylated c-Met showed no correlation with survival of prostate cancer patients.

### REFERENCES

Blair, H. C., Teitelbaum, S. L., Gbiselli, R., & Gluck, S. (1989). Osteoclastic bone resorption by a polarized vacuolar proton pump. Science, 245(4920), 855-857.
Chen, G., Sircar, K., Aprikian, A., Potti, A., Goltzman, D., &Rabbani, S. A. (2006). Expression of RANKL RANK/OPG in primary and metastatic human prostate cancer as markers of disease stage and functional regulation. Cancer, 107(2), 289-298.
Landis, S. H., Murray, T., Bolden, S., &Wingo, P. A. (1999). Cancer statistics, 1999. CA Cancer J Clin, 49(1), 8-31, 31.
Li, Y., Kucuk, 0., Hussain, M., Abrams, J., Cher, M. L., &Sarkar, F. H. (2006). Antitumor and antimetastatic activities of docetaxel are enhanced by genistein through regulation of osteoprotegerin/receptor activator of nuclear factor-kappaB (RANK)/RANK ligand/IVIMP-9 signaling in prostate cancer. Cancer Res, 66(9), 4816-4825.
Mundy, G. R. (2002). Metastasis to bone: causes, consequences and therapeutic opportunities, Nat Rev Cancer, 2(8), 584-593.
Roodman, G. D. (1999). Cell biology of the osteoclast. Exp Hematol, 27(8), 12291241.
Roodman, G. D. (2004). Mechanisms of bone metastasis. N Engl. J Med, 350(16), 1655-1664.
Xu, J., Wang, R., Xie, Z. H., Odero-Marah, V., Pathak, S., Multani, A., et al. (2006). Prostate cancer metastasis: role of the host microenvironment in promoting epithelial to mesenchymal transition and increased bone and adrenal gland metastasis. Prostate, 66(15), 1664-1673.
Yasuda, H., Shima, N., Nakagawa, N., Yamaguchi, K., Kinosaki, M., Mochizuki, S., et al. (1998). Osteoclast differentiation factor is a ligand for osteoprotegerin/osteoclastogenesis-inhibitory factor and is identical to TRANCE/RANKL. Proc Nati Acad Sci USA, 95(7), 3597-3602.
Ott, S. M. (2008). Histomorphometric measurements of bone turnover, mineralization, and volume. Clin J Am Soc Nephrol, 3 Suppl 3, S151-156.
Zhang, S., Zhau, H. E., Osunkoya, A. O., Iqbal, S., Yang, X., Fan, S., et al. Vascular endothelial growth factor regulates myeloid cell leukemia-1 expression through neuropilin-1-dependent activation of c-MET signaling in human prostate cancer cells. Mol Cancer, 9, 9.
1. Lipton A, Uzzo R, Amato R J, Ellis G K, Hakimian B, Roodman G D, and Smith M R (2009). The science and practice of bone health in oncology: managing bone loss and metastasis in patients with solid tumors. J Natl Compr Canc Netw 7 Suppl 7, S1-29; quiz S30.
2. Tu S M, and Lin S H (2008). Current trials using bone-targeting agents in prostate cancer. Cancer J 14, 35-39.
3. Josson S, Matsuoka Y, Chung L W, Zhau H E, and Wang R (2010). Tumor-stroma co-evolution in prostate cancer progression and metastasis. Semin Cell Dev Biol 21, 26-32.
4. Mundy G R (1997). Mechanisms of bone metastasis. Cancer 80, 1546-1556.
5. Coleman R E (1997). Skeletal complications of malignancy. Cancer 80, 1588-1594.
6. Roodman G D (2010). Pathogenesis of myeloma bone disease. J Cell Biochem 109, 283-291.
7. Schramek D, Leibbrandt A, Sigl V, Kenner L, Pospisilik J A, Lee H J, Hanada R, Joshi P A, Aliprantis A, Glimcher L, et al. (2010). Osteoclast differentiation factor RANKL controls development of progestin-driven mammary cancer. Nature 468, 98-102.
8. Lydon J P (2010). Stem cells: Cues from steroid hormones. Nature 465, 695-696.
9. Joshi P A, Jackson H W, Beristain A G, Di Grappa M A, Mote P A, Clarke C L, Stingl J, Waterhouse P D, and Khokha R (2010). Progesterone induces adult mammary stem cell expansion. Nature 465, 803-807.
10. Asselin-Labat M L, Vaillant F, Sheridan J M, Pal B, Wu D, Simpson E R, Yasuda H, Smyth G K, Martin T J, Lindeman G J, et al. (2010). Control of mammary stem cell function by steroid hormone signalling. Nature 465, 798-802.
11. Kostenuik P J, Nguyen H Q, McCabe J, Warmington K S, Kurahara C, Sun N, Chen C, Li L, Cattley R C, Van G, et al. (2009). Denosumab, a fully human monoclonal antibody to RANKL, inhibits bone resorption and increases BMD in knock-in mice that express chimeric (murine/human) RANKL. J Bone Miner Res 24, 182-195.
12. Dougall W, and Chaisson M (2006). [Monoclonal antibody targeting RANKL as a therapy for cancer-induced bone diseases]. Clin Calcium 16, 627-635.
13. Smith D, Spira A, Holbrecht S, Lin C, and al. e (2010). Phase 2 study of XL184 in a cohort of patients with castration resistant prostate cancer. EORTC, 406a.
14. Chung L W, Huang W C, Sung S Y, Wu D, Odero-Marah V, Nomura T, Shigemura K, Miyagi T, Seo S, Shi C, et al. (2006). Stromal-epithelial interaction in prostate cancer progression. Clin Genitourin Cancer 5, 162-170.
15. Josson S, Nomura T, Lin J T, Huang W C, Wu D, Zhau H E, Zayzafoon M, Weizmann M N, Gurirajan M, and Chung L W (2011). Beta2-microglobulin induces epithelial to mesenchymal transition and confoers cancer lethality and bone metastasis in human cancer cells. Cancer Research (in press).
16. Koeneman K S, Yeung F, and Chung L W (1999). Osteomimetic properties of prostate cancer cells: a hypothesis supporting the predilection of prostate cancer metastasis and growth in the bone environment. Prostate 39, 246-261.
17. Rucci N, and Teti A (2010). Osteomimicry: how tumor cells try to deceive the bone. Front Biosci (Schol Ed) 2, 907-915.
18. Barnett P, Arnold R S, Mezencev R, Chung L W, Zayzafoon M, and Odero-Marah V (2011). Snail-mediated regulation of reactive oxygen species in ARCaP human prostate cancer cells. Biochem Biophys Res Commun 404, 34-39.
19. Odero-Marah V A, Wang R, Chu G, Zayzafoon M, Xu J, Shi C, Marshall F F, Zhau H E, and Chung L W (2008). Receptor activator of NF-kappaB Ligand (RANKL) expression is associated with epithelial to mesenchymal transition in human prostate cancer cells. Cell Res 18, 858-870.
20. Xu J, Wang R, Xie Z H, Odero-Marah V, Pathak S, Multani A, Chung L W, and Zhau H E (2006). Prostate cancer metastasis: role of the host microenvironment in promoting epithelial to mesenchymal transition and increased bone and adrenal gland metastasis. Prostate 66, 1664-1673.
21. Zhau H E, Odero-Marah V, Lue H W, Nomura T, Wang R, Chu G, Liu Z R, Zhou B P, Huang W C, and Chung L W (2008). Epithelial to mesenchymal transition (EMT) in human prostate cancer: lessons learned from ARCaP model. Clin Exp Metastasis 25, 601-610.
22. Thalmann G N, Anezinis P E, Chang S M, Zhau H E, Kim E E, Hopwood V L, Pathak S, von Eschenbach A C, and Chung L W (1994). Androgen-independent cancer progression and bone metastasis in the LNCaP model of human prostate cancer. Cancer Res 54, 2577-2581.
23. He H, Yang X, Davidson A J, Wu D, Marshall F F, Chung L W, Zhau H E, and Wang R (2010). Progressive epithelial to mesenchymal transitions in ARCaP E prostate cancer cells during xenograft tumor formation and metastasis. Prostate 70, 518-528.
24. Wu H C, Hsieh J T, Gleave M E, Brown N M, Pathak S, and Chung L W (1994). Derivation of androgen-independent human LNCaP prostatic cancer cell sublines: role of bone stromal cells. Int J Cancer 57, 406-412.
25. Gleave M, Hsieh J T, Gao C A, von Eschenbach A C, and Chung L W (1991). Acceleration of human prostate cancer growth in vivo by factors produced by prostate and bone fibroblasts. Cancer Res 51, 3753-3761.
26. Gleave M E, Hsieh J T, von Eschenbach A C, and Chung L W (1992). Prostate and bone fibroblasts induce human prostate cancer growth in vivo: implications for bidirectional tumor-stromal cell interaction in prostate carcinoma growth and metastasis. J Urol 147, 1151-1159.
27. Hsieh J T, Wu H C, Gleave M E, von Eschenbach A C, and Chung L W (1993). Autocrine regulation of prostate-specific antigen gene expression in a human prostatic cancer (LNCaP) subline. Cancer Res 53, 2852-2857.
28. Zhang S, Zhau H E, Osunkoya A O, Iqbal S, Yang X, Fan S, Chen Z, Wang R, Marshall F F, Chung L W, et al. (2010). Vascular endothelial growth factor regulates myeloid cell leukemia-1 expression through neuropilin-1-dependent activation of c-MET signaling in human prostate cancer cells. Mol Cancer 9, 9.
29. Yu Y Y, Lieu S, Lu C, and Colnot C (2010). Bone morphogenetic protein 2 stimulates endochondral ossification by regulating periosteal cell fate during bone repair. Bone 47, 65-73.
30. Cackowski F C, Anderson J L, Patrene K D, Choksi R J, Shapiro S D, Windle J J, Blair H C, and Roodman G D (2010). Osteoclasts are important for bone angiogenesis. Blood 115, 140-149.
31. Punzi L, Podswiadek M, Sfriso P, Oliviero F, Fiocco U, and Todesco S (2007). Pathogenetic and clinical rationale for TNF-blocking therapy in psoriatic arthritis. Autoimmun Rev 6, 524-528.
32. Kiesel J, Miller C, Abu-Amer Y, and Aurora R (2007). Systems level analysis of osteoclastogenesis reveals intrinsic and extrinsic regulatory interactions. Dev Dyn 236, 2181-2197.
33. Tanaka Y, Abe M, Hiasa M, Oda A, Amou H, Nakano A, Takeuchi K, Kitazoe K, Kido S, Inoue D, et al. (2007). Myeloma cell-osteoclast interaction enhances angiogenesis together with bone resorption: a role for vascular endothelial cell growth factor and osteopontin. Clin Cancer Res 13, 816-823.
34. Menu E, De Leenheer E, De Raeve H, Coulton L, Imanishi T, Miyashita K, Van Valckenborgh E, Van Riet I, Van Camp B, Horuk R, et al. (2006). Role of CCR1 and CCR5 in homing and growth of multiple myeloma and in the development of osteolytic lesions: a study in the 5TMM model. Clin Exp Metastasis 23, 291-300.
35. Cenni E, Perut F, Granchi D, Amato I, Avnet S, Brandi M L, Giunti A, and Baldini N (2006). Primitive and bone metastatic renal carcinoma cells promote osteoclastogenesis through endothelial cells. Anticancer Res 26, 3065-3069.
36. Niida S, Kondo T, Hiratsuka S, Hayashi S, Amizuka N, Noda T, Ikeda K, and Shibuya M (2005). VEGF receptor 1 signaling is essential for osteoclast development and bone marrow formation in colony-stimulating factor 1-deficient mice. Proc Natl Acad Sci USA 102, 14016-14021.
37. Chikatsu N, Takeuchi Y, Fukumoto S, Yano K, Fujita N, Tsuruo T, and Fujita T (2002). Clonal endothelial cells produce humoral factors that inhibit osteoclast-like cell formation in vitro. Endocr J 49, 439-447.
38. Asou Y, Rittling S R, Yoshitake H, Tsuji K, Shinomiya K, Nifuji A, Denhardt D T, and Noda M (2001). Osteopontin facilitates angiogenesis, accumulation of osteoclasts, and resorption in ectopic bone. Endocrinology 142, 1325-1332.
39. Georgiou K R, Foster B K, and Xian C J (2010). Damage and recovery of the bone marrow microenvironment induced by cancer chemotherapy—potential regulatory role of chemokine CXCL12/receptor CXCR4 signalling. Curr Mol Med 10, 440-453.
40. Shiozawa Y, Jung Y, Ziegler A M, Pedersen E A, Wang J, Wang Z, Song J, Lee C H, Sud S, Pienta K J, et al. (2010). Erythropoietin couples hematopoiesis with bone formation. PLoS One 5, e10853.
41. Liu J, Zhao Z, Zou L, Li J, Wang F, Li X, Zhang J, Liu Y, Chen S, Zhi M, et al. (2009). Pressure-loaded MSCs during early osteodifferentiation promote osteoclastogenesis by increase of RANKL/OPG ratio. Ann Biomed Eng 37, 794-802.
42. Jiang Y, Mishima H, Sakai S, Liu Y K, Ohyabu Y, and Uemura T (2008). Gene expression analysis of major lineage-defining factors in human bone marrow cells: effect of aging, gender, and age-related disorders. J Orthop Res 26, 910-917.
43. Yang D C, Tsay H J, Lin S Y, Chiou S H, Li M J, Chang T J, and Hung S C (2008). cAMP/PKA regulates osteogenesis, adipogenesis and ratio of RANKL/OPG mRNA expression in mesenchymal stem cells by suppressing leptin. PLoS One 3, e1540.
44. Zhang Y, Guessous F, Kofman A, Schiff D, and Abounader R (2010). XL-184, a MET, VEGFR-2 and RET kinase inhibitor for the treatment of thyroid cancer, glioblastoma multiforme and NSCLC. IDrugs 13, 112-121.
45. Tanaka H, Kono E, Tran C P, Miyazaki H, Yamashiro J, Shimomura T, Fazli L, Wada R, Huang J, Vessella R L, et al. (2010). Monoclonal antibody targeting of N-cadherin inhibits prostate cancer growth, metastasis and castration resistance. Nat Med 16, 1414-1420.
46. Armstrong A P, Miller R E, Jones J C, Zhang J, Keller E T, and Dougall W C (2008). RANKL acts directly on RANK-expressing prostate tumor cells and mediates migration and expression of tumor metastasis genes. Prostate 68, 92-104.
47. Zhang J, Dai J, Qi Y, Lin D L, Smith P, Strayhorn C, Mizokami A, Fu Z, Westman J, and Keller E T (2001). Osteoprotegerin inhibits prostate cancer-induced osteoclastogenesis and prevents prostate tumor growth in the bone. J Clin Invest 107, 1235-1244.
48. Morrissey C, Kostenuik P L, Brown L G, Vessella R L, and Corey E (2007). Host-derived RANKL is responsible for osteolysis in a C4-2 human prostate cancer xenograft model of experimental bone metastases. BMC Cancer 7, 148.
49. Magalhaes J K, Grynpas M D, Willett T L, and Glogauer M (2010). Deleting Racl improves vertebral bone quality and resistance to fracture in a murine ovariectomy model. Osteoporos Int.
50. Taylor K H, Middlefell L S, and Mizen K D (2010). Osteonecrosis of the jaws induced by anti-RANK ligand therapy. Br J Oral Maxillofac Surg 48, 221-223.
51. Savore C, Zhang C, Muir C, Liu R, Wyrwa J, Shu J, Zhau H E, Chung L W, Carson D D, and Farach-Carson M C (2005). Perlecan knockdown in metastatic prostate cancer cells reduces heparin-binding growth factor responses in vitro and tumor growth in vivo. Clin Exp Metastasis 22, 377-390.
52. Goto T, Matsui Y, Fernandes R J, Hanson D A, Kubo T, Yukata K, Michigami T, Komori T, Fujita T, Yang L, et al. (2006). Sp 1 family of transcription factors regulates the human alpha2 (XI) collagen gene (COL11A2) in Saos-2 osteoblastic cells. J Bone Miner Res 21, 661-673.
53. Jackson R S, 2nd, Franco O E, and Bhowmick N A (2008). Gene targeting to the stroma of the prostate and bone. Differentiation 76, 606-623.
54. Boomershine C S, Chamberlain A, Kendall P, Afshar-Sharif A R, Huang H, Washington M K, Lawson W E, Thomas J W, Blackwell T S, and Bhowmick N A (2009). Autoimmune pancreatitis results from loss of TGFbeta signalling in S100A4-positive dendritic cells. Gut 58, 1267-1274.
55. Placencio V R, Sharif-Afshar A R, Li X, Huang H, Uwamariya C, Neilson E G, Shen M M, Matusik R J, Hayward S W, and Bhowmick N A (2008). Stromal transforming growth factor-beta signaling mediates prostatic response to androgen ablation by paracrine Wnt activity. Cancer Res 68, 4709-4718.
56. Yang X, Shi C, Tong R, Qian W, Zhau H E, Wang R, Zhu G, Cheng J, Yang V W, Cheng T, et al. (2010). Near IR heptamethine cyanine dye-mediated cancer imaging. Clin Cancer Res 16, 2833-2844.
57. Hsieh C L, Xie Z, Yu J, Martin W D, Datta M W, Wu G J, and Chung L W (2007). Non-invasive bioluminescent detection of prostate cancer growth and metastasis in a bigenic transgenic mouse model. Prostate 67, 685-691.
58. Hsieh C L, Xie Z, Liu Z Y, Green J E, Martin W D, Datta M W, Yeung F, Pan D, and Chung L W (2005). A luciferase transgenic mouse model: visualization of prostate development and its androgen responsiveness in live animals. J Mol Endocrinol 35, 293-304.
59. Hsieh C L, Gardner T A, Miao L, Balian G, and Chung L W (2004). Cotargeting tumor and stroma in a novel chimeric tumor model involving the growth of both human prostate cancer and bone stromal cells. Cancer Gene Ther 11, 148-155.
60. Sun X, He H, Xie Z, Qian W, Zhau H E, Chung L W, Marshall F F, and Wang R (2010). Matched pairs of human prostate stromal cells display differential tropic effects on LNCaP prostate cancer cells. In Vitro Cell Dev Biol Anim 46, 538-546.
61. Bisanz K, Yu J, Edlund M, Spohn B, Hung M C, Chung L W, and Hsieh C L (2005). Targeting ECM-integrin interaction with liposome-encapsulated small interfering RNAs inhibits the growth of human prostate cancer in a bone xenograft imaging model. Mol Ther 12, 634-643.
62. Kramer I, Halleux C, Keller H, Pegurri M, Gooi J H, Weber P B, Feng J Q, Bonewald L F, and Kneissel M (2010). Osteocyte Wnt/beta-catenin signaling is required for normal bone homeostasis. Mol Cell Biol 30, 3071-3085.
63. Zhou Z, Han J Y, Xi C X, Xie J X, Feng X, Wang C Y, Mei L, and Xiong W C (2008). HMGB1 regulates RANKL-induced osteoclastogenesis in a manner dependent on RAGE. J Bone Miner Res 23, 1084-1096.
64. Sung S Y, Hsieh C L, Law A, Zhau H E, Pathak S, Multani A S, Lim S, Coleman I M, Wu L C, Figg W D, et al. (2008). Coevolution of prostate cancer and bone stroma in three-dimensional coculture: implications for cancer growth and metastasis. Cancer Res 68, 9996-10003.
65. Rhee H W, Zhau H E, Pathak S, Multani A S, Pennanen S, Visakorpi T, and Chung L W (2001). Permanent phenotypic and genotypic changes of prostate cancer cells cultured in a three-dimensional rotating-wall vessel. In Vitro Cell Dev Biol Anim 37, 127-140.
66. Yates C, Shepard C R, Papworth G, Dash A, Beer Stolz D, Tannenbaum S, Griffith L, and Wells A (2007). Novel three-dimensional organotypic liver bioreactor to directly visualize early events in metastatic progression. Adv Cancer Res 97, 225-246.
67. Jessberger S, Clemenson G D, Jr., and Gage F H (2007). Spontaneous fusion and nonclonal growth of adult neural stem cells. Stem Cells 25, 871-874.
68. Kaposi-Novak P, Lee J S, Gomez-Quiroz L, Coulouarn C, Factor V M, and Thorgeirsson S S (2006). Met-regulated expression signature defines a subset of human hepatocellular carcinomas with poor prognosis and aggressive phenotype. J Clin Invest 116, 1582-1595.
69. Gotte M, Kersting C, Radke I, Kiesel L, and Wulfing P (2007). An expression signature of syndecan-1 (CD138), E-cadherin and c-met is associated with factors of angiogenesis and lymphangiogenesis in ductal breast carcinoma in situ. Breast Cancer Res 9, R8.
70. Hakozaki A, Yoda M, Tohmonda T, Furukawa M, Hikata T, Uchikawa S, Takaishi H, Matsumoto M, Chiba K, Horiuchi K, et al. (2010). Receptor activator of NF-kappaB (RANK) ligand induces ectodomain shedding of RANK in murine RAW264.7 macrophages. J Immunol 184, 2442-2448.
71. Owen K A, Qiu D, Alves J, Schumacher A M, Kilpatrick L M, Li J, Harris J L, and Ellis V (2010). Pericellular activation of hepatocyte growth factor by the transmembrane serine proteases matriptase and hepsin, but not by the membrane-associated protease uPA. Biochem J 426, 219-228.
72. Qiu D, Owen K, Gray K, Bass R, and Ellis V (2007). Roles and regulation of membrane-associated serine proteases. Biochem Soc Trans 35, 583-587.
73. Tatsumi R, and Allen R E (2004). Active hepatocyte growth factor is present in skeletal muscle extracellular matrix. Muscle Nerve 30, 654-658.
74. Somerset D A, Strain A J, Afford S, Whittle M J, and Kilby M D (2000). Hepatocyte growth factor activator (HGF-A) and its zymogen in human placenta. Placenta 21, 615-620.
75. Shimomura T, Denda K, Kawaguchi T, Matsumoto K, Miyazawa K, and Kitamura N (1999). Multiple sites of proteolytic cleavage to release soluble forms of hepatocyte growth factor activator inhibitor type 1 from a transmembrane form. J Biochem 126, 821-828.
76. McGonigle J S, Giachelli C M, and Scatena M (2009). Osteoprotegerin and RANKL differentially regulate angiogenesis and endothelial cell function. Angiogenesis 12, 35-46.
77. Min J K, Cho Y L, Choi J H, Kim Y, Kim J H, Yu Y S, Rho J, Mochizuki N, Kim Y M, Oh G T, et al. (2007). Receptor activator of nuclear factor (NF)-kappaB ligand (RANKL) increases vascular permeability: impaired permeability and angiogenesis in eNOS-deficient mice. Blood 109, 1495-1502.
78. Sulpice E, Ding S, Muscatelli-Groux B, Berge M, Han Z C, Plouet J, Tobelem G, and Merkulova-Rainon T (2009). Cross-talk between the VEGF-A and HGF signalling pathways in endothelial cells. Biol Cell 101, 525-539.
79. Lin J W, Sheu W H, Lee W J, Chen Y T, Liu T J, Ting C T, and Lee W L (2007). Circulating hepatocyte growth factor level but not basic fibroblast growth factor level is elevated in angiography-proven symptomatic peripheral artery disease. Angiology 58, 420-428.
80. Ieda Y, Fujita J, Ieda M, Yagi T, Kawada H, Ando K, and Fukuda K (2007). G-CSF and HGF: combination of vasculogenesis and angiogenesis synergistically improves recovery in murine hind limb ischemia. J Mol Cell Cardiol 42, 540-548.
81. Riggins K S, Mernaugh G, Su Y, Quaranta V, Koshikawa N, Seiki M, Pozzi A, and Zent R (2010). MT1-MMP-mediated basement membrane remodeling modulates renal development. Exp Cell Res 316, 2993-3005.
82. Ortega N, Behonick D, Stickens D, and Werb Z (2003). How proteases regulate bone morphogenesis. Ann N Y Acad Sci 995, 109-116.
83. Cole M D, and Nikiforov M A (2006). Transcriptional activation by the Myc oncoprotein. Curr Top Microbiol Immunol 302, 33-50.
84. Denhardt D T (1996). Oncogene-initiated aberrant signaling engenders the metastatic phenotype: synergistic transcription factor interactions are targets for cancer therapy. Crit Rev Oncog 7, 261-291.
85. Huang W C, Zhau H E, and Chung L W (2010). Androgen receptor survival signaling is blocked by anti-beta2-microglobulin monoclonal antibody via a MAPK/lipogenic pathway in human prostate cancer cells. J Biol Chem 285, 7947-7956.
86. Brar S S, Corbin Z, Kennedy T P, Hemendinger R, Thornton L, Bommarius B, Arnold R S, Whorton A R, Sturrock A B, Huecksteadt T P, et al. (2003). NOX5 NAD(P)H oxidase regulates growth and apoptosis in DU 145 prostate cancer cells. Am J Physiol Cell Physiol 285, C353-369.
87. Wang C, Yeung F, Liu P C, Attar R M, Geng J, Chung L W, Gottardis M, and Kao C (2003). Identification of a novel transcription factor, GAGATA-binding protein, involved in androgen-mediated expression of prostate-specific antigen. J Biol Chem 278, 32423-32430.
88. Yeung F, and Chung L W (2002). Molecular basis of co-targeting prostate tumor and stroma. J Cell Biochem Suppl 38, 65-72.
89. Yeung F, Law W K, Yeh C H, Westendorf J J, Zhang Y, Wang R, Kao C, and Chung L W (2002). Regulation of human osteocalcin promoter in hormone-independent human prostate cancer cells. J Biol Chem 277, 2468-2476.
90. Yeung F, Li X, Ellett J, Trapman J, Kao C, and Chung L W (2000). Regions of prostate-specific antigen (PSA) promoter confer androgen-independent expression of PSA in prostate cancer cells. J Biol Chem 275, 40846-40855.
91. Nordstrand A, Nilsson J, Tieva A, Wikstrom P, Lerner U H, and Widmark A (2009). Establishment and validation of an in vitro co-culture model to study the interactions between bone and prostate cancer cells. Clin Exp Metastasis 26, 945-953.
92. Mizutani K, Sud S, and Pienta K J (2009). Prostate cancer promotes CD11b positive cells to differentiate into osteoclasts. J Cell Biochem 106, 563-569.
93. Fizazi K, Yang J, Peleg S, Sikes C R, Kreimann E L, Daliani D, Olive M, Raymond K A, Janus T J, Logothetis C J, et al. (2003). Prostate cancer cells-osteoblast interaction shifts expression of growth/survival-related genes in prostate cancer and reduces expression of osteoprotegerin in osteoblasts. Clin Cancer Res 9, 2587-2597.
94. Lum L, Wong B R, Josien R, Becherer J D, Erdjument-Bromage H, Schlondorff J, Tempst P, Choi Y, and Blobel C P (1999). Evidence for a role of a tumor necrosis factor-alpha (TNF-alpha)-converting enzyme-like protease in shedding of TRANCE, a TNF family member involved in osteoclastogenesis and dendritic cell survival. J Biol Chem 274, 13613-13618.
95. Hikita A, Yana I, Wakeyama H, Nakamura M, Kadono Y, Oshima Y, Nakamura K, Seiki M, and Tanaka S (2006). Negative regulation of osteoclastogenesis by ectodomain shedding of receptor activator of NF-kappaB ligand. J Biol Chem 281, 36846-36855.
96. Johnson F M, and Gallick G E (2007). SRC family nonreceptor tyrosine kinases as molecular targets for cancer therapy. Anticancer Agents Med Chem 7, 651-659.
97. Fizazi K (2007). The role of Src in prostate cancer. Ann Oncol 18, 1765-1773.
98. Chang Y M, Kung H J, and Evans C P (2007). Nonreceptor tyrosine kinases in prostate cancer. Neoplasia 9, 90-100.
99. Singh P, Uzgare A, Litvinov I, Denmeade S R, and Isaacs J T (2006). Combinatorial androgen receptor targeted therapy for prostate cancer. Endocr Relat Cancer 13, 653-666.
100. Swinnen J V, Brusselmans K, and Verhoeven G (2006). Increased lipogenesis in cancer cells: new players, novel targets. Curr Opin Clin Nutr Metab Care 9, 358-365.
101. Heemers H V, Verhoeven G, and Swinnen J V (2006). Androgen activation of the sterol regulatory element-binding protein pathway: Current insights. Mol Endocrinol 20, 2265-2277.
102. Etherton T D, Louveau I, Sorensen M T, and Chaudhuri S (1993). Mechanisms by which somatotropin decreases adipose tissue growth. Am J Clin Nutr 58, 287S-295S.
103. Calmy A, Fux C A, Norris R, Vallier N, Delhumeau C, Samaras K, Hesse K, Hirschel B, Cooper D A, and Carr A (2009). Low bone mineral density, renal dysfunction, and fracture risk in HIV infection: a cross-sectional study. J Infect Dis 200, 1746-1754.
104. Seitz S, Schnabel C, Busse B, Schmidt H U, Beil F T, Friedrich R E, Schinke T, Mautner V F, and Amling M (2010). High bone turnover and accumulation of osteoid in patients with neurofibromatosis 1. Osteoporos Int 21, 119-127.
105. Miller P D (2009). Denosumab: anti-RANKL antibody. Curr Osteoporos Rep 7, 18-22.
106. Zhau H E, Hong S J, and Chung L W (1994). A fetal rat urogenital sinus mesenchymal cell line (rUGM): accelerated growth and conferral of androgen-induced growth responsiveness upon a human bladder cancer epithelial cell line in vivo. Int J Cancer 56, 706-714.
107. Gleave M E, Hsieh J T, Wu H C, von Eschenbach A C, and Chung L W (1992). Serum prostate specific antigen levels in mice bearing human prostate LNCaP tumors are determined by tumor volume and endocrine and growth factors. Cancer Res 52, 1598-1605.
108. Shigemura K, Arbiser J L, Sun S Y, Zayzafoon M, Johnstone P A, Fujisawa M, Gotoh A, Weksler B, Zhau H E, and Chung L W (2007). Honokiol, a natural plant product, inhibits the bone metastatic growth of human prostate cancer cells. Cancer 109, 1279-1289.
109. Thalmann G N, Sikes R A, Chang S M, Johnston D A, von Eschenbach A C, and Chung L W (1996). Suramin-induced decrease in prostate-specific antigen expression with no effect on tumor growth in the LNCaP model of human prostate cancer. J Natl Cancer Inst 88, 794-801.
110. Jin F, Xie Z, Kuo C J, Chung L W, and Hsieh C L (2005). Cotargeting tumor and tumor endothelium effectively inhibits the growth of human prostate cancer in adenovirus-mediated antiangiogenesis and oncolysis combination therapy. Cancer Gene Ther 12, 257-267.
111. Liu T, Xue R, Dong L, Wu H, Zhang D, and Shen X (2011). Rapid determination of serological cytokine biomarkers for hepatitis B virus-related hepatocellular carcinoma using antibody microarrays. Acta Biochim Biophys Sin (Shanghai) 43, 45-51.
112. Hon K L, Ching G K, Wong K Y, Leung T F, and Leung A K (2008). A pilot study to explore the usefulness of antibody array in childhood atopic dermatitis. J Natl Med Assoc 100, 500-504.
113. Bantscheff M, Hopf C, Kruse U, and Drewes G (2007). Proteomics-based strategies in kinase drug discovery. Ernst Schering Found Symp Proc, 1-28.
114. Chaerkady R, and Pandey A (2007). Quantitative proteomics for identification of cancer biomarkers. Proteomics Clin Appl 1, 1080-1089.
115. Pimienta G, Chaerkady R, and Pandey A (2009). SILAC for global phosphoproteomic analysis. Methods Mol Biol 527, 107-116, x.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A method for treating cancer in a mammalian subject in need thereof, comprising:
providing an agent capable of inhibiting RANK and/or RANKL, and an agent capable of inhibiting HGF-c-Met/VEGFR2/neuropilin-1-mediated signaling; and
administering the agent capable of inhibiting RANK and/or RANKL and the agent capable of inhibiting HGF-c-Met/VEGFR2/neuropilin-1-mediated signaling to the mammalian subject to treat cancer.

2. The method of claim 1, wherein the agent capable of inhibiting RANK and/or RANKL is provided in a first composition and the agent capable of inhibiting HGF-c-Met/VEGFR2/neuropilin-1-mediated signaling is provided in a second composition.

3. The method of claim 1, wherein the agent capable of inhibiting RANK and/or RANKL and the agent capable of inhibiting HGF-c-Met/VEGFR2/neuropilin-1-mediated signaling are provided in one composition.

4. The method of claim 1, wherein the agent capable of inhibiting RANK and/or RANKL is denosumab, RANK-Fc, OPG-Fc, shRNA, or siRNA.

5. (canceled)

6. The method of claim 1, wherein the agent capable of inhibiting RANK and/or RANKL is denosumab.

7. The method of claim 1, wherein the agent capable of inhibiting HGF-c-Met/VEGFR2/neuropilin-1-mediated signaling is denosumab, RANK-Fc, OPG-Fc, shRNA, siRNA, crizotinib, or VEGFR2 kinase inhibitor III (CAS 204005-46-9).

8. (canceled)

9. The method of claim 1, wherein the cancer is prostate, kidney, breast, bladder, lung, breast, ovarian, pancreatic, thyroid, liver, gastric, colon or melanoma.

10. The method of claim 1, wherein the cancer is prostate cancer.

11. The method of claim 1, wherein the inhibiting HGF-c-Met/VEGFR2/neuropilin-1-mediated signaling comprises inhibiting activation of c-Met, VEGFR2, neuropilin-1, Src-kinase, Stat3, Mcl-1, NF-kB or combinations thereof.

12. A method of preventing, reducing the likelihood of and/or inhibiting metastases of cancer cells, comprising:
providing a composition comprising an agent capable of inhibiting epithelial-to-mesenchymal transition (EMT) of cancer cells; and
administering a quantity of the composition to the a mammalian subject in need thereof to prevent, reduce the likelihood of and/or inhibit metastases of cancer cells.

13. The method of claim 12, wherein the agent capable of inhibiting EMT is osteoprotegerin (OPG) and binds to RANKL to inhibit the formation of osteoclasts, thereby preventing, reducing the likelihood and/or inhibiting metastases of the cancer cells.

14. The method of claim 12, wherein the agent capable of inhibiting EMT is denosumab, RANK-Fc, OPG-Fc, siRNA, shRNA, crizotinib, or VEGFR2 kinase inhibitor III (CAS 204005-46-9) or combinations thereof.

15. (canceled)

16. The method of claim 12, wherein the cancer cells are prostate, kidney, breast, bladder, lung, ovarian, pancreatic, thyroid, liver, gastric, colon or melanoma cancer cells.

17. The method according to claim 12, wherein the cancer cells are prostate cancer cells.

18. A method of inhibiting a process of RANKL-mediated awakening of cancer dormancy, comprising:
providing a composition comprising an agent capable of inhibiting epithelial-to-mesenchymal transition (EMT) of cancer cells; and
administering a quantity of the composition to the a mammalian subject in need thereof to inhibiting the process of RANKL-mediated awakening of cancer dormancy.

19. The method of claim 18, wherein the agent capable of inhibiting EMT is osteoprotegerin (OPG) and binds to RANKL to inhibit the formation of osteoclasts, thereby inhibiting the process of RANKL-mediated awakening of cancer dormancy.

20. The method of claim 18, wherein the agent capable of inhibiting EMT is denosumab, RANK-Fc, OPG-Fc, siRNA, shRNA, crizotinib, or VEGFR2 kinase inhibitor III (CAS 204005-46-9) or combinations thereof.

21. (canceled)

22. The method of claim 18, wherein the cancer is prostate, kidney, breast, bladder, lung, ovarian, pancreatic, thyroid, liver, gastric, colon or melanoma cancer.

23. The method according to claim 18, wherein the cancer is prostate cancer.

24. A cell expressing a target selected from the group consisting of RANKL, an EMT marker, NF-kB, c-Met, VEGFR2, neuropilin-1, Src-kinase, Stat3, Mcl-1, and combinations thereof.

25. A method of identifying a compound that inhibits metastasis, comprising:
providing the cell of claim 24;
contacting the cell with a test compound; and
determining whether metastasis is inhibited in the presence of the test compound,
wherein the decrease of the expression of the target is an indication that the test compound inhibits metastasis or
wherein the decrease of a target's upstream signaling components, Src-kinase or Stat3 phosphorylation is an indication that the test compound inhibits metastasis.

26. The method of claim 25, wherein the EMT marker is selected from the group consisting of N-cadherin, vimentin, VEGF, RANKL, c-Met and combinations thereof.

27. (canceled)

28. The method of claim 25, wherein the cell is a prostate, kidney, breast, bladder, lung, ovarian, pancreatic, thyroid, liver, gastric, colon or melanoma cancer cell.

29. (canceled)

30. The method of claim 28, wherein the prostate cancer cell is ARCaP_{E}, ARCaP_{M}, C4-2, LNCaP, PC3 or MCF7.

31. An animal, comprising the cell of claim 24.

32. The animal of claim 31, wherein the cell is an LNCaP-RANKL cell.

33. The animal of claim 31, wherein the animal is a mouse.

34. A method of identifying a compound that inhibits metastasis, comprising:
providing the animal of claim 31;
contacting the animal with a test compound; and
determining whether metastasis is inhibited in the presence of the test compound.

35. The method of claim 34, wherein the decrease of the expression of the target is an indication that the test compound inhibits metastasis or wherein the decrease of a target's upstream signaling components, Src-kinase or Stat3 phosphorylation is an indication that the test compound inhibits metastasis.

36. The method of claim 34, wherein the decrease of metastasis of the cancer in the animal is an indication that the test compound inhibits metastasis.

37. A method of switching osteolytic bone lesion and/or metastasis to osteoblastic bone lesion and/or metastasis in a subject in need thereof comprising:
providing an agent capable of blocking RANK/RANKL signaling and/or HGF/cMet/VEGF/VEGFR2/neuropilin-1-mediated signaling; and
administering the agent to the subject to switch osteolytic bone lesion and/or metastasis to osteoblastic bone lesion and/or metastasis.

38. The method of claim 37, wherein the agent is selected from the group consisting of denosumab, RANK-Fc, OPG-Fc, siRNA, shRNA, crizotinib, VEGFR2 kinase inhibitor III (CAS 204005-46-9) and combinations thereof.

39-77. (canceled)
